SEQUENCE LISTING (1360 sequences)

SEQ ID NO: 1-10 - skipped (10 entries)

SEQ ID NO: 11 - protein, 26 aa, synthetic construct
  source 1..26: note = FR-H1
QVQLVQSGAEVKKPGASVKVSCKASG

SEQ ID NO: 12 - protein, 9 aa, synthetic construct
  source 1..9: note = CDR-H1
FNIKDYYMH

SEQ ID NO: 13 - protein, 14 aa, synthetic construct
  source 1..14: note = FR-H2
WVRQAPGQRLEWMG

SEQ ID NO: 14 - protein, 17 aa, synthetic construct
  source 1..17: note = CDR-H2
WIDLENANTIYDAKFQG

SEQ ID NO: 15 - protein, 30 aa, synthetic construct
  source 1..30: note = FR-H3
  VAR_SEQ 16: note = X is Glu or Gly
RVTITRDTSASTAYMXLSSLRSEDTAVYYC

SEQ ID NO: 16 - protein, 12 aa, synthetic construct
  source 1..12: note = CDR-H3
  VAR_SEQ 4: note = X is Ala or His
  VAR_SEQ 6: note = X is His or Gly
ARDXYXRYFYDV

SEQ ID NO: 17 - protein, 11 aa, synthetic construct
  source 1..11: note = FR-H4
WGQGTLVTVSS

SEQ ID NO: 18-20 - skipped (3 entries)

SEQ ID NO: 21 - protein, 23 aa, synthetic construct
  source 1..23: note = FR-L1
  VAR_SEQ 1: note = X is Asp or Gly
XIVMTQSPDSLAVSLGERATINC

SEQ ID NO: 22 - protein, 17 aa, synthetic construct
  source 1..17: note = CDR-L1
  VAR_SEQ 13: note = X is His or Lys
KSSQSLLNARTGXNYLA

SEQ ID NO: 23 - protein, 15 aa, synthetic construct
  source 1..15: note = FR-L2
WYQQKPGQPPKLLIY

SEQ ID NO: 24 - protein, 7 aa, synthetic construct
  source 1..7: note = CDR-L2
WASTRES

SEQ ID NO: 25 - protein, 32 aa, synthetic construct
  source 1..32: note = FR-L3
GVPDRFSGSGSGTDFTLTISSLQAEDVAVYYC

SEQ ID NO: 26 - protein, 8 aa, synthetic construct
  source 1..8: note = CDR-L3
  VAR_SEQ 4: note = X is Tyr or His
  VAR_SEQ 6: note = X is His or Arg
KQSXSXRT

SEQ ID NO: 27 - protein, 10 aa, synthetic construct
  source 1..10: note = FR-L4
FGGGTKVEIK

SEQ ID NO: 28-40 - skipped (13 entries)

SEQ ID NO: 41 - protein, 98 aa, synthetic construct
  source 1..98: note = Human IgG1 CH1, reference 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 42 - protein, 98 aa, synthetic construct
  source 1..98: note = Human IgG1 CH1, reference 2
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 43-50 - skipped (8 entries)

SEQ ID NO: 51 - protein, 15 aa, synthetic construct
  source 1..15: note = Human IgG1 hinge, reference
EPKSCDKTHTCPPCP

SEQ ID NO: 52-60 - skipped (9 entries)

SEQ ID NO: 61 - protein, 110 aa, synthetic construct
  source 1..110: note = Human IgG1 CH2, reference
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 62-70 - skipped (9 entries)

SEQ ID NO: 71 - protein, 106 aa, synthetic construct [sequence omitted]

SEQ ID NO: 72 - protein, 106 aa, synthetic construct
  source 1..106: note = Human IgG1 CH3 (356E and 358M)
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 106 aa, synthetic construct
  source 1..106: note = Human IgG1 CH3 (356D, 358L, and V422I)
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 74 - protein, 106 aa, synthetic construct
  source 1..106: note = Human IgG1 CH3 (356D, 358L, and A431G)
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 75-80 - skipped (6 entries)

SEQ ID NO: 81 - protein, 107 aa, synthetic construct [sequence omitted]

SEQ ID NO: 82 - protein, 106 aa, synthetic construct
  source 1..106: note = Human IgG CL
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83-90 - skipped (8 entries)

SEQ ID NO: 91 - protein, 335 aa, synthetic construct
  source 1..335: note = Hu CD3epsilon Fc
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 326 aa, synthetic construct
  source 1..326: note = Cy CD3epsilon Fc
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93-109 - skipped (17 entries)

SEQ ID NO: 110 - protein, 119 aa, synthetic construct
  source 1..119: note = VH ADI-26906
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 111 - protein, 26 aa, synthetic construct
  source 1..26: note = ADI-26906
QVQLVQSGAEVKKPGASVKVSCKASG

SEQ ID NO: 112 - protein, 9 aa, synthetic construct
  source 1..9: note = ADI-26906
FNIKDYYMH

SEQ ID NO: 113 - protein, 14 aa, synthetic construct
  source 1..14: note = ADI-26906
WVRQAPGQRLEWMG

SEQ ID NO: 114 - protein, 17 aa, synthetic construct
  source 1..17: note = ADI-26906
WIDLENANTIYDAKFQG

SEQ ID NO: 115 - protein, 30 aa, synthetic construct
  source 1..30: note = ADI-26906
RVTITRDTSASTAYMELSSLRSEDTAVYYC

SEQ ID NO: 116 - protein, 12 aa, synthetic construct
  source 1..12: note = ADI-26906
ARDAYGRYFYDV

SEQ ID NO: 117 - protein, 11 aa, synthetic construct
  source 1..11: note = ADI-26906
WGQGTLVTVSS

SEQ ID NO: 118-119 - skipped (2 entries)

SEQ ID NO: 120 - protein, 112 aa, synthetic construct
  source 1..112: note = VL ADI-26906
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 121 - protein, 23 aa, synthetic construct
  source 1..23: note = ADI-26906
DIVMTQSPDSLAVSLGERATINC

SEQ ID NO: 122 - protein, 17 aa, synthetic construct
  source 1..17: note = ADI-26906
KSSQSLLNARTGKNYLA

SEQ ID NO: 123 - protein, 15 aa, synthetic construct
  source 1..15: note = ADI-26906
WYQQKPGQPPKLLIY

SEQ ID NO: 124 - protein, 7 aa, synthetic construct
  source 1..7: note = ADI-26906
WASTRES

SEQ ID NO: 125 - protein, 32 aa, synthetic construct; identical to SEQ ID NO: 25
  source 1..32: note = ADI-26906

SEQ ID NO: 126 - protein, 8 aa, synthetic construct
  source 1..8: note = ADI-26906
KQSYSRRT

SEQ ID NO: 127 - protein, 10 aa, synthetic construct
  source 1..10: note = ADI-26906
FGGGTKVEIK

SEQ ID NO: 128-149 - skipped (22 entries)

SEQ ID NO: 150 - DNA, 357 bp, synthetic construct
  source 1..357: mol_type = other DNA; note = VH ADI-26906
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 151-159 - skipped (9 entries)

SEQ ID NO: 160 - DNA, 336 bp, synthetic construct
  source 1..336: mol_type = other DNA; note = VL ADI-26906
gacatcgtgatgacccagtctccagactccctggctgtgtctctgggcgagagggccaccatcaactgcaagtccagccagagtcttttaaacgctagaaccggaaagaactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatcttattctcggagaactttcggcggagggaccaaggtcgagatcaaa

SEQ ID NO: 161-209 - skipped (49 entries)

SEQ ID NO: 210 - protein, 119 aa, synthetic construct
  source 1..119: note = VH ADI-48587
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDHYHRYFYDVWGQGTLVTVSS

SEQ ID NO: 211 - protein, 26 aa, synthetic construct
  source 1..26: note = ADI-48587
QVQLVQSGAEVKKPGASVKVSCKASG

SEQ ID NO: 212 - protein, 9 aa, synthetic construct
  source 1..9: note = ADI-48587
FNIKDYYMH

SEQ ID NO: 213 - protein, 14 aa, synthetic construct
  source 1..14: note = ADI-48587
WVRQAPGQRLEWMG

SEQ ID NO: 214 - protein, 17 aa, synthetic construct
  source 1..17: note = ADI-48587
WIDLENANTIYDAKFQG

SEQ ID NO: 215 - protein, 30 aa, synthetic construct; identical to SEQ ID NO: 115
  source 1..30: note = ADI-48587

SEQ ID NO: 216 - protein, 12 aa, synthetic construct
  source 1..12: note = ADI-48587
ARDHYHRYFYDV

SEQ ID NO: 217 - protein, 11 aa, synthetic construct
  source 1..11: note = ADI-48587
WGQGTLVTVSS

SEQ ID NO: 218-219 - skipped (2 entries)

SEQ ID NO: 220 - protein, 112 aa, synthetic construct
  source 1..112: note = VL ADI-48587
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSHSHRTFGGGTKVEIK

SEQ ID NO: 221 - protein, 23 aa, synthetic construct
  source 1..23: note = ADI-48587
DIVMTQSPDSLAVSLGERATINC

SEQ ID NO: 222 - protein, 17 aa, synthetic construct
  source 1..17: note = ADI-48587
KSSQSLLNARTGHNYLA

SEQ ID NO: 223 - protein, 15 aa, synthetic construct
  source 1..15: note = ADI-48587
WYQQKPGQPPKLLIY

SEQ ID NO: 224 - protein, 7 aa, synthetic construct
  source 1..7: note = ADI-48587
WASTRES

SEQ ID NO: 225 - protein, 32 aa, synthetic construct; identical to SEQ ID NO: 25
  source 1..32: note = ADI-48587

SEQ ID NO: 226 - protein, 8 aa, synthetic construct
  source 1..8: note = ADI-48587
KQSHSHRT

SEQ ID NO: 227 - protein, 10 aa, synthetic construct
  source 1..10: note = ADI-48587
FGGGTKVEIK

SEQ ID NO: 228-249 - skipped (22 entries)

SEQ ID NO: 250 - DNA, 357 bp, synthetic construct
  source 1..357: mol_type = other DNA; note = VH ADI-48587
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatcattaccatagatatttttacgacgtgtggggccaaggaaccctggtcaccgtctcctca

SEQ ID NO: 251-259 - skipped (9 entries)

SEQ ID NO: 260 - DNA, 336 bp, synthetic construct
  source 1..336: mol_type = other DNA; note = VL ADI-48587
gacatcgtgatgacccagtctccagactccctggctgtgtctctgggcgagagggccaccatcaactgcaagtccagccagagtcttttaaacgctagaaccggacataactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatctcattctcatagaactttcggcggagggaccaaggttgagatcaaa

SEQ ID NO: 261-309 - skipped (49 entries)

SEQ ID NO: 310 - protein, 119 aa, synthetic construct
  source 1..119: note = VH ADI-74965
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYHRYFYDVWGQGTLVTVSS

SEQ ID NO: 311 - protein, 26 aa, synthetic construct
  source 1..26: note = ADI-74965
QVQLVQSGAEVKKPGASVKVSCKASG

SEQ ID NO: 312 - protein, 9 aa, synthetic construct
  source 1..9: note = ADI-74965
FNIKDYYMH

SEQ ID NO: 313 - protein, 14 aa, synthetic construct
  source 1..14: note = ADI-74965
WVRQAPGQRLEWMG

SEQ ID NO: 314 - protein, 17 aa, synthetic construct
  source 1..17: note = ADI-74965
WIDLENANTIYDAKFQG

SEQ ID NO: 315 - protein, 30 aa, synthetic construct; identical to SEQ ID NO: 115
  source 1..30: note = ADI-74965

SEQ ID NO: 316 - protein, 12 aa, synthetic construct
  source 1..12: note = ADI-74965
ARDAYHRYFYDV

SEQ ID NO: 317 - protein, 11 aa, synthetic construct
  source 1..11: note = ADI-74965
WGQGTLVTVSS

SEQ ID NO: 318-319 - skipped (2 entries)

SEQ ID NO: 320 - protein, 112 aa, synthetic construct; identical to SEQ ID NO: 220
  source 1..112: note = VL ADI-74965

SEQ ID NO: 321 - protein, 23 aa, synthetic construct
  source 1..23: note = ADI-74965
DIVMTQSPDSLAVSLGERATINC

SEQ ID NO: 322 - protein, 17 aa, synthetic construct
  source 1..17: note = ADI-74965
KSSQSLLNARTGHNYLA

SEQ ID NO: 323 - protein, 15 aa, synthetic construct
  source 1..15: note = ADI-74965
WYQQKPGQPPKLLIY

SEQ ID NO: 324 - protein, 7 aa, synthetic construct
  source 1..7: note = ADI-74965
WASTRES

SEQ ID NO: 325 - protein, 32 aa, synthetic construct; identical to SEQ ID NO: 25
  source 1..32: note = ADI-74965

SEQ ID NO: 326 - protein, 8 aa, synthetic construct
  source 1..8: note = ADI-74965
KQSHSHRT

SEQ ID NO: 327 - protein, 10 aa, synthetic construct
  source 1..10: note = ADI-74965
FGGGTKVEIK

SEQ ID NO: 328-349 - skipped (22 entries)

SEQ ID NO: 350 - DNA, 357 bp, synthetic construct
  source 1..357: mol_type = other DNA; note = VH ADI-74965
caggtgcagctggtgcagtctggggctgaggtgaagaagcctggggcctcagtgaaggtctcctgcaaggcttccggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatgcgtaccatagatatttttacgacgtgtggggccaaggaaccctggtcaccgtctcctca

SEQ ID NO: 351-359 - skipped (9 entries)

SEQ ID NO: 360 - DNA, 336 bp, synthetic construct; identical to SEQ ID NO: 260
  source 1..336: mol_type = other DNA; note = VL ADI-74965

SEQ ID NO: 361-409 - skipped (49 entries)

SEQ ID NO: 410 - protein, 119 aa, synthetic construct; identical to SEQ ID NO: 210
  source 1..119: note = VH ADI-74966

SEQ ID NO: 411 - protein, 26 aa, synthetic construct
  source 1..26: note = ADI-74966
QVQLVQSGAEVKKPGASVKVSCKASG

SEQ ID NO: 412 - protein, 9 aa, synthetic construct
  source 1..9: note = ADI-74966
FNIKDYYMH

SEQ ID NO: 413 - protein, 14 aa, synthetic construct
  source 1..14: note = ADI-74966
WVRQAPGQRLEWMG

SEQ ID NO: 414 - protein, 17 aa, synthetic construct
  source 1..17: note = ADI-74966
WIDLENANTIYDAKFQG

SEQ ID NO: 415 - protein, 30 aa, synthetic construct; identical to SEQ ID NO: 115
  source 1..30: note = ADI-74966

SEQ ID NO: 416 - protein, 12 aa, synthetic construct
  source 1..12: note = ADI-74966
ARDHYHRYFYDV

SEQ ID NO: 417 - protein, 11 aa, synthetic construct
  source 1..11: note = ADI-74966
WGQGTLVTVSS

SEQ ID NO: 418-419 - skipped (2 entries)

SEQ ID NO: 420 - protein, 112 aa, synthetic construct
  source 1..112: note = VL ADI-74966
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSYSHRTFGGGTKVEIK

SEQ ID NO: 421 - protein, 23 aa, synthetic construct
  source 1..23: note = ADI-74966
DIVMTQSPDSLAVSLGERATINC

SEQ ID NO: 422 - protein, 17 aa, synthetic construct
  source 1..17: note = ADI-74966
KSSQSLLNARTGHNYLA

SEQ ID NO: 423 - protein, 15 aa, synthetic construct
  source 1..15: note = ADI-74966
WYQQKPGQPPKLLIY

SEQ ID NO: 424 - protein, 7 aa, synthetic construct
  source 1..7: note = ADI-74966
WASTRES

SEQ ID NO: 425 - protein, 32 aa, synthetic construct; identical to SEQ ID NO: 25
  source 1..32: note = ADI-74966

SEQ ID NO: 426 - protein, 8 aa, synthetic construct
  source 1..8: note = ADI-74966
KQSYSHRT

SEQ ID NO: 427 - protein, 10 aa, synthetic construct
  source 1..10: note = ADI-74966
FGGGTKVEIK

SEQ ID NO: 428-449 - skipped (22 entries)

SEQ ID NO: 450 - DNA, 357 bp, synthetic construct; identical to SEQ ID NO: 250
  source 1..357: mol_type = other DNA; note = VH ADI-74966

SEQ ID NO: 451-459 - skipped (9 entries)

SEQ ID NO: 460 - DNA, 336 bp, synthetic construct
  source 1..336: mol_type = other DNA; note = VL ADI-74966
gacatcgtgatgacccagtctccagactccctggctgtgtctctgggcgagagggccaccatcaactgcaagtccagccagagtcttttaaacgctagaaccggacataactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatcttactctcatagaactttcggcggagggaccaaggttgagatcaaa

SEQ ID NO: 461-509 - skipped (49 entries)

SEQ ID NO: 510 - protein, 119 aa, synthetic construct; identical to SEQ ID NO: 210
  source 1..119: note = VH ADI-74967

SEQ ID NO: 511 - protein, 26 aa, synthetic construct
  source 1..26: note = ADI-74967
QVQLVQSGAEVKKPGASVKVSCKASG

SEQ ID NO: 512 - protein, 9 aa, synthetic construct
  source 1..9: note = ADI-74967
FNIKDYYMH

SEQ ID NO: 513 - protein, 14 aa, synthetic construct
  source 1..14: note = ADI-74967
WVRQAPGQRLEWMG

SEQ ID NO: 514 - protein, 17 aa, synthetic construct
  source 1..17: note = ADI-74968
WIDLENANTIYDAKFQG

SEQ ID NO: 515 - protein, 30 aa, synthetic construct; identical to SEQ ID NO: 115
  source 1..30: note = ADI-74967

SEQ ID NO: 516 - protein, 12 aa, synthetic construct
  source 1..12: note = ADI-74967
ARDHYHRYFYDV

SEQ ID NO: 517 - protein, 11 aa, synthetic construct
  source 1..11: note = ADI-74967
WGQGTLVTVSS

SEQ ID NO: 518-519 - skipped (2 entries)

SEQ ID NO: 520 - protein, 112 aa, synthetic construct
  source 1..112: note = VL ADI-74967
GIVMTQSPDSLAVSLGERATINCKSSQSLLNARTGKNYLAWYQQKPGQPPKLLIYWASTRESGVPDRFSGSGSGTDFTLTISSLQAEDVAVYYCKQSHSHRTFGGGTKVEIK

SEQ ID NO: 521 - protein, 23 aa, synthetic construct
  source 1..23: note = ADI-74967
GIVMTQSPDSLAVSLGERATINC

SEQ ID NO: 522 - protein, 17 aa, synthetic construct
  source 1..17: note = ADI-74967
KSSQSLLNARTGKNYLA

SEQ ID NO: 523 - protein, 15 aa, synthetic construct
  source 1..15: note = ADI-74967
WYQQKPGQPPKLLIY

SEQ ID NO: 524 - protein, 7 aa, synthetic construct
  source 1..7: note = ADI-74967
WASTRES

SEQ ID NO: 525 - protein, 32 aa, synthetic construct; identical to SEQ ID NO: 25
  source 1..32: note = ADI-74967

SEQ ID NO: 526 - protein, 8 aa, synthetic construct
  source 1..8: note = ADI-74967
KQSHSHRT

SEQ ID NO: 527 - protein, 10 aa, synthetic construct
  source 1..10: note = ADI-74967
FGGGTKVEIK

SEQ ID NO: 528-549 - skipped (22 entries)

SEQ ID NO: 550 - DNA, 357 bp, synthetic construct; identical to SEQ ID NO: 250
  source 1..357: mol_type = other DNA; note = VH ADI-74967

SEQ ID NO: 551-559 - skipped (9 entries)

SEQ ID NO: 560 - DNA, 336 bp, synthetic construct
  source 1..336: mol_type = other DNA; note = VL ADI-74967
ggcatcgtgatgacccagtctccagactccctggctgtgtctctgggcgagagggccaccatcaactgcaagtccagccagagtcttttaaacgctagaaccggaaagaactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatctcattctcatagaactttcggcggggggaccaaggttgagatcaaa

SEQ ID NO: 561-609 - skipped (49 entries)

SEQ ID NO: 610 - protein, 119 aa, synthetic construct; identical to SEQ ID NO: 310
  source 1..119: note = VH ADI-74968

SEQ ID NO: 611 - protein, 26 aa, synthetic construct
  source 1..26: note = ADI-74968
QVQLVQSGAEVKKPGASVKVSCKASG

SEQ ID NO: 612 - protein, 9 aa, synthetic construct
  source 1..9: note = ADI-74968
FNIKDYYMH

SEQ ID NO: 613 - protein, 14 aa, synthetic construct
  source 1..14: note = ADI-74968
WVRQAPGQRLEWMG

SEQ ID NO: 614 - protein, 17 aa, synthetic construct
  source 1..17: note = ADI-74968
WIDLENANTIYDAKFQG

SEQ ID NO: 615 - protein, 30 aa, synthetic construct; identical to SEQ ID NO: 115
  source 1..30: note = ADI-74968

SEQ ID NO: 616 - protein, 12 aa, synthetic construct
  source 1..12: note = ADI-74968
ARDAYHRYFYDV

SEQ ID NO: 617 - protein, 11 aa, synthetic construct
  source 1..11: note = ADI-74968
WGQGTLVTVSS

SEQ ID NO: 618-619 - skipped (2 entries)

SEQ ID NO: 620 - protein, 112 aa, synthetic construct; identical to SEQ ID NO: 420
  source 1..112: note = VL ADI-74968

SEQ ID NO: 621 - protein, 23 aa, synthetic construct
  source 1..23: note = ADI-74968
DIVMTQSPDSLAVSLGERATINC

SEQ ID NO: 622 - protein, 17 aa, synthetic construct
  source 1..17: note = ADI-74968
KSSQSLLNARTGHNYLA

SEQ ID NO: 623 - protein, 15 aa, synthetic construct
  source 1..15: note = ADI-74968
WYQQKPGQPPKLLIY

SEQ ID NO: 624 - protein, 7 aa, synthetic construct
  source 1..7: note = ADI-74968
WASTRES

SEQ ID NO: 625 - protein, 32 aa, synthetic construct; identical to SEQ ID NO: 25
  source 1..32: note = ADI-74968

SEQ ID NO: 626 - protein, 8 aa, synthetic construct
  source 1..8: note = ADI-74968
KQSYSHRT

SEQ ID NO: 627 - protein, 10 aa, synthetic construct
  source 1..10: note = ADI-74968
FGGGTKVEIK

SEQ ID NO: 628-649 - skipped (22 entries)

SEQ ID NO: 650 - DNA, 357 bp, synthetic construct
  source 1..357: mol_type = other DNA; note = VH ADI-74968
caggtccagcttgtgcagtctggggctgaggtgaagaagcctggggcctcagtgaaggtttcctgcaaggcttctggatttaatattaaagactattacatgcattgggtgcgccaggcccccggacaaaggcttgagtggatgggatggatcgatttggagaacgcgaacacaatctatgacgcaaagttccagggcagagtcaccattaccagggacacatccgcgagcacagcctacatggagctgagcagcctgagatctgaagacacggcggtgtactactgcgccagggacgcctaccatcgatacttttatgacgtttggggccagggcaccctggtcaccgtctcctca

SEQ ID NO: 651-659 - skipped (9 entries)

SEQ ID NO: 660 - DNA, 336 bp, synthetic construct
  source 1..336: mol_type = other DNA; note = VL ADI-74968
gacatcgtgatgacccagtctccagactccctggctgtgtctctgggcgagagggccaccatcaactgcaagtccagccagagtctcttaaacgcacgtacaggtcataactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtaagcagtcttacagtcatcgcacattcggcggagggaccaaggtggagatcaaa

SEQ ID NO: 661-709 - skipped (49 entries)

SEQ ID NO: 710 - protein, 119 aa, synthetic construct
  source 1..119: note = VH ADI-79842
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDHYGRYFYDVWGQGTLVTVSS

SEQ ID NO: 711 - protein, 26 aa, synthetic construct
  source 1..26: note = ADI-79842
QVQLVQSGAEVKKPGASVKVSCKASG

SEQ ID NO: 712 - protein, 9 aa, synthetic construct
  source 1..9: note = ADI-79842
FNIKDYYMH

SEQ ID NO: 713 - protein, 14 aa, synthetic construct
  source 1..14: note = ADI-79842
WVRQAPGQRLEWMG

SEQ ID NO: 714 - protein, 17 aa, synthetic construct
  source 1..17: note = ADI-79842
WIDLENANTIYDAKFQG

SEQ ID NO: 715 - protein, 30 aa, synthetic construct; identical to SEQ ID NO: 115
  source 1..30: note = ADI-79842

SEQ ID NO: 716 - protein, 12 aa, synthetic construct
  source 1..12: note = ADI-79842
ARDHYGRYFYDV

SEQ ID NO: 717 - protein, 11 aa, synthetic construct
  source 1..11: note = ADI-79842
WGQGTLVTVSS

SEQ ID NO: 718-719 - skipped (2 entries)

SEQ ID NO: 720 - protein, 112 aa, synthetic construct; identical to SEQ ID NO: 120
  source 1..112: note = VL ADI-79842

SEQ ID NO: 721 - protein, 23 aa, synthetic construct
  source 1..23: note = ADI-79842
DIVMTQSPDSLAVSLGERATINC

SEQ ID NO: 722 - protein, 17 aa, synthetic construct
  source 1..17: note = ADI-79842
KSSQSLLNARTGKNYLA

SEQ ID NO: 723 - protein, 15 aa, synthetic construct
  source 1..15: note = ADI-79842
WYQQKPGQPPKLLIY

SEQ ID NO: 724 - protein, 7 aa, synthetic construct
  source 1..7: note = ADI-79842
WASTRES

SEQ ID NO: 725 - protein, 32 aa, synthetic construct; identical to SEQ ID NO: 25
  source 1..32: note = ADI-79842

SEQ ID NO: 726 - protein, 8 aa, synthetic construct
  source 1..8: note = ADI-79842
KQSYSRRT

SEQ ID NO: 727 - protein, 10 aa, synthetic construct
  source 1..10: note = ADI-79842
FGGGTKVEIK

SEQ ID NO: 728-749 - skipped (22 entries)

SEQ ID NO: 750 - DNA, 357 bp, synthetic construct
  source 1..357: mol_type = other DNA; note = VH ADI-79842
caggtgcagctggtgcagtctggggctgaggtgaagaagcctggggcctcagtgaaggtctcctgcaaggcttctggattcaacatcaaggactactatatgcactgggtgagacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatcactacgggagatatttttacgacgtgtggggccaaggaaccctggtcaccgtctcctca

SEQ ID NO: 751-759 - skipped (9 entries)

SEQ ID NO: 760 - DNA, 336 bp, synthetic construct
  source 1..336: mol_type = other DNA; note = VL ADI-79842
gacatcgtgatgacccagtctccagactccctggctgtgtctctgggcgagagggccaccatcaactgcaagtccagccagagtcttttgaacgctagaaccggaaagaactacctggcttggtaccagcagaaaccaggacagcctcctaagctgttgatttactgggcatctaccagagaatccggggtccctgacagattcagtggcagcgggtctgggacagacttcactttgactatcagtagcctgcaggctgaagatgtggcagtttattactgtaagcaatcttattctagaagaactttcggcggagggaccaaggtcgagatcaaa

SEQ ID NO: 761-809 - skipped (49 entries)

SEQ ID NO: 810 - protein, 119 aa, synthetic construct; identical to SEQ ID NO: 310
  source 1..119: note = VH ADI-79843

SEQ ID NO: 811 - protein, 26 aa, synthetic construct
  source 1..26: note = ADI-79843
QVQLVQSGAEVKKPGASVKVSCKASG

SEQ ID NO: 812 - protein, 9 aa, synthetic construct
  source 1..9: note = ADI-79843
FNIKDYYMH

SEQ ID NO: 813 - protein, 14 aa, synthetic construct
  source 1..14: note = ADI-79843
WVRQAPGQRLEWMG

SEQ ID NO: 814 - protein, 17 aa, synthetic construct
  source 1..17: note = ADI-79843
WIDLENANTIYDAKFQG

SEQ ID NO: 815 - protein, 30 aa, synthetic construct; identical to SEQ ID NO: 115
  source 1..30: note = ADI-79843

SEQ ID NO: 816 - protein, 12 aa, synthetic construct
  source 1..12: note = ADI-79843
ARDAYHRYFYDV

SEQ ID NO: 817 - protein, 11 aa, synthetic construct
  source 1..11: note = ADI-79843
WGQGTLVTVSS

SEQ ID NO: 818-819 - skipped (2 entries)

SEQ ID NO: 820 - protein, 112 aa, synthetic construct; identical to SEQ ID NO: 120
  source 1..112: note = VL ADI-79843

SEQ ID NO: 821 - protein, 23 aa, synthetic construct
  source 1..23: note = ADI-79843
DIVMTQSPDSLAVSLGERATINC

SEQ ID NO: 822 - protein, 17 aa, synthetic construct
  source 1..17: note = ADI-79843
KSSQSLLNARTGKNYLA

SEQ ID NO: 823 - protein, 15 aa, synthetic construct
  source 1..15: note = ADI-79843
WYQQKPGQPPKLLIY

SEQ ID NO: 824 - protein, 7 aa, synthetic construct
  source 1..7: note = ADI-79843
WASTRES

SEQ ID NO: 825 - protein, 32 aa, synthetic construct; identical to SEQ ID NO: 25
  source 1..32: note = ADI-79843

SEQ ID NO: 826 - protein, 8 aa, synthetic construct
  source 1..8: note = ADI-79843
KQSYSRRT

SEQ ID NO: 827 - protein, 10 aa, synthetic construct
  source 1..10: note = ADI-79843
FGGGTKVEIK

SEQ ID NO: 828-849 - skipped (22 entries)

SEQ ID NO: 850 - DNA, 357 bp, synthetic construct
  source 1..357: mol_type = other DNA; note = VH ADI-79843
caggtgcagctggtgcagtctggggctgaggtgaagaagcctggggcctcagtgaaggtctcctgcaaggcttctggattcaacatcaaggactactatatgcactgggtgaggcaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatgcttaccatagatatttttacgacgtgtggggccaaggaaccctggtcaccgtctcctca

SEQ ID NO: 851-859 - skipped (9 entries)

SEQ ID NO: 860 - DNA, 336 bp, synthetic construct; identical to SEQ ID NO: 760
  source 1..336: mol_type = other DNA; note = VL ADI-79843

SEQ ID NO: 861-909 - skipped (49 entries)

SEQ ID NO: 910 - protein, 119 aa, synthetic construct; identical to SEQ ID NO: 110
  source 1..119: note = VH ADI-79848

SEQ ID NO: 911 - protein, 26 aa, synthetic construct
  source 1..26: note = ADI-79848
QVQLVQSGAEVKKPGASVKVSCKASG

SEQ ID NO: 912 - protein, 9 aa, synthetic construct
  source 1..9: note = ADI-79848
FNIKDYYMH

SEQ ID NO: 913 - protein, 14 aa, synthetic construct
  source 1..14: note = ADI-79848
WVRQAPGQRLEWMG

SEQ ID NO: 914 - protein, 17 aa, synthetic construct
  source 1..17: note = ADI-79848
WIDLENANTIYDAKFQG

SEQ ID NO: 915 - protein, 30 aa, synthetic construct; identical to SEQ ID NO: 115
  source 1..30: note = ADI-79848

SEQ ID NO: 916 - protein, 12 aa, synthetic construct
  source 1..12: note = ADI-79848
ARDAYGRYFYDV

SEQ ID NO: 917 - protein, 11 aa, synthetic construct
  source 1..11: note = ADI-79848
WGQGTLVTVSS

SEQ ID NO: 918-919 - skipped (2 entries)

SEQ ID NO: 920 - protein, 112 aa, synthetic construct
  source 1..112: note = VL ADI-79848
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSYSRRTFGGGTKVEIK

SEQ ID NO: 921 - protein, 23 aa, synthetic construct
  source 1..23: note = ADI-79848
DIVMTQSPDSLAVSLGERATINC

SEQ ID NO: 922 - protein, 17 aa, synthetic construct
  source 1..17: note = ADI-79848
KSSQSLLNARTGHNYLA

SEQ ID NO: 923 - protein, 15 aa, synthetic construct
  source 1..15: note = ADI-79848
WYQQKPGQPPKLLIY

SEQ ID NO: 924 - protein, 7 aa, synthetic construct
  source 1..7: note = ADI-79848
WASTRES

SEQ ID NO: 925 - protein, 32 aa, synthetic construct; identical to SEQ ID NO: 25
  source 1..32: note = ADI-79848

SEQ ID NO: 926 - protein, 8 aa, synthetic construct
  source 1..8: note = ADI-79848
KQSYSRRT

SEQ ID NO: 927 - protein, 10 aa, synthetic construct
  source 1..10: note = ADI-79848
FGGGTKVEIK

SEQ ID NO: 928-949 - skipped (22 entries)

SEQ ID NO: 950 - DNA, 357 bp, synthetic construct
  source 1..357: mol_type = other DNA; note = VH ADI-79848
caggtgcagctggtgcagtctggggctgaggtgaagaagcctggggcctcagtgaaggtctcctgcaaggcttctggattcaacatcaaggactactatatgcactgggtgaggcaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatgcttacgggagatatttttacgacgtgtggggccaaggaaccctggtcaccgtctcctca

SEQ ID NO: 951-959 - skipped (9 entries)

SEQ ID NO: 960 - DNA, 336 bp, synthetic construct
  source 1..336: mol_type = other DNA; note = VL ADI-79848
gacatcgtgatgacccagtctccagactccctggctgtgtctctgggcgagagggccaccatcaactgcaagtccagccagagtcttttgaacgctagaaccggacataactacctggcttggtaccagcagaaaccaggacagcctcctaagctgctgatttactgggcatctaccagagaatccggggtccctgacagattcagtggcagcgggtctgggacagacttcactttgactatcagtagcctgcaggctgaagatgtggcagtttattactgtaagcaatcttattctaggagaactttcggcggagggaccaaggtcgagatcaaa

SEQ ID NO: 961-1009 - skipped (49 entries)

SEQ ID NO: 1010 - protein, 119 aa, synthetic construct; identical to SEQ ID NO: 110
  source 1..119: note = VH ADI-79844

SEQ ID NO: 1011 - protein, 26 aa, synthetic construct
  source 1..26: note = ADI-79844
QVQLVQSGAEVKKPGASVKVSCKASG

SEQ ID NO: 1012 - protein, 9 aa, synthetic construct
  source 1..9: note = ADI-79844
FNIKDYYMH

SEQ ID NO: 1013 - protein, 14 aa, synthetic construct
  source 1..14: note = ADI-79844
WVRQAPGQRLEWMG

SEQ ID NO: 1014 - protein, 17 aa, synthetic construct
  source 1..17: note = ADI-79844
WIDLENANTIYDAKFQG

SEQ ID NO: 1015 - protein, 30 aa, synthetic construct; identical to SEQ ID NO: 115
  source 1..30: note = ADI-79844

SEQ ID NO: 1016 - protein, 12 aa, synthetic construct
  source 1..12: note = ADI-79844
ARDAYGRYFYDV

SEQ ID NO: 1017 - protein, 11 aa, synthetic construct
  source 1..11: note = ADI-79844
WGQGTLVTVSS

SEQ ID NO: 1018-1019 - skipped (2 entries)

SEQ ID NO: 1020 - protein, 112 aa, synthetic construct
  source 1..112: note = VL ADI-79844
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSHSRRTFGGGTKVEIK

SEQ ID NO: 1021 - protein, 23 aa, synthetic construct
  source 1..23: note = ADI-79844
DIVMTQSPDSLAVSLGERATINC

SEQ ID NO: 1022 - protein, 17 aa, synthetic construct
  source 1..17: note = ADI-79844
KSSQSLLNARTGKNYLA

SEQ ID NO: 1023 - protein, 15 aa, synthetic construct
  source 1..15: note = ADI-79844
WYQQKPGQPPKLLIY

SEQ ID NO: 1024 - protein, 7 aa, synthetic construct
  source 1..7: note = ADI-79844
WASTRES

SEQ ID NO: 1025 - protein, 32 aa, synthetic construct; identical to SEQ ID NO: 25
  source 1..32: note = ADI-79844

SEQ ID NO: 1026 - protein, 8 aa, synthetic construct
  source 1..8: note = ADI-79844
KQSHSRRT

SEQ ID NO: 1027 - protein, 10 aa, synthetic construct
  source 1..10: note = ADI-79844
FGGGTKVEIK

SEQ ID NO: 1028-1049 - skipped (22 entries)

SEQ ID NO: 1050 - DNA, 357 bp, synthetic construct; identical to SEQ ID NO: 950
  source 1..357: mol_type = other DNA; note = VH ADI-79844

SEQ ID NO: 1051-1059 - skipped (9 entries)

SEQ ID NO: 1060 - DNA, 336 bp, synthetic construct
  source 1..336: mol_type = other DNA; note = VL ADI-79844
gacatcgtgatgacccagtctccagactccctggctgtgtctctgggcgagagggccaccatcaactgcaagtccagccagagtcttttgaacgctagaaccggaaagaactacttggcttggtaccagcagaaaccaggacagcctcctaagctgctgatttactgggcatctaccagagaatccggggtccctgacagattcagtggcagcgggtctgggacagacttcactttgactatcagtagcctgcaggctgaagatgtggcagtttattactgtaagcaatctcactctagaagaactttcggcggagggaccaaggtcgagatcaaa

SEQ ID NO: 1061-1109 - skipped (49 entries)

SEQ ID NO: 1110 - protein, 119 aa, synthetic construct; identical to SEQ ID NO: 110
  source 1..119: note = VH ADI-79845

SEQ ID NO: 1111 - protein, 26 aa, synthetic construct
  source 1..26: note = ADI-79845
QVQLVQSGAEVKKPGASVKVSCKASG

SEQ ID NO: 1112 - protein, 9 aa, synthetic construct
  source 1..9: note = ADI-79845
FNIKDYYMH

SEQ ID NO: 1113 - protein, 14 aa, synthetic construct
  source 1..14: note = ADI-79845
WVRQAPGQRLEWMG

SEQ ID NO: 1114 - protein, 17 aa, synthetic construct
  source 1..17: note = ADI-79845
WIDLENANTIYDAKFQG

SEQ ID NO: 1115 - protein, 30 aa, synthetic construct; identical to SEQ ID NO: 115
  source 1..30: note = ADI-79845

SEQ ID NO: 1116 - protein, 12 aa, synthetic construct
  source 1..12: note = ADI-79845
ARDAYGRYFYDV

SEQ ID NO: 1117 - protein, 11 aa, synthetic construct
  source 1..11: note = ADI-79845
WGQGTLVTVSS

SEQ ID NO: 1118-1119 - skipped (2 entries)

SEQ ID NO: 1120 - protein, 112 aa, synthetic construct
  source 1..112: note = VL ADI-79845
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSHRTFGGGTKVEIK

SEQ ID NO: 1121 - protein, 23 aa, synthetic construct
  source 1..23: note = ADI-79845
DIVMTQSPDSLAVSLGERATINC

SEQ ID NO: 1122 - protein, 17 aa, synthetic construct
  source 1..17: note = ADI-79845
KSSQSLLNARTGKNYLA

SEQ ID NO: 1123 - protein, 15 aa, synthetic construct
  source 1..15: note = ADI-79845
WYQQKPGQPPKLLIY

SEQ ID NO: 1124 - protein, 7 aa, synthetic construct
  source 1..7: note = ADI-79845
WASTRES

SEQ ID NO: 1125 - protein, 32 aa, synthetic construct; identical to SEQ ID NO: 25
  source 1..32: note = ADI-79845

SEQ ID NO: 1126 - protein, 8 aa, synthetic construct
  source 1..8: note = ADI-79845
KQSYSHRT

SEQ ID NO: 1127 - protein, 10 aa, synthetic construct
  source 1..10: note = ADI-79845
FGGGTKVEIK

SEQ ID NO: 1128-1149 - skipped (22 entries)

SEQ ID NO: 1150 - DNA, 357 bp, synthetic construct; identical to SEQ ID NO: 950
  source 1..357: mol_type = other DNA; note = VH ADI-79845

SEQ ID NO: 1151-1159 - skipped (9 entries)

SEQ ID NO: 1160 - DNA, 336 bp, synthetic construct
  source 1..336: mol_type = other DNA; note = VL ADI-79845
gacatcgtgatgacccagtctccagactccctggctgtgtctctgggcgagagggccaccatcaactgcaagtccagccagagtcttcttaacgctagaaccggaaagaactacctggcttggtaccagcagaaaccaggacagcctcctaagctgcttatttactgggcatctaccagggaatccggggtccctgacagattcagtggcagcgggtctgggacagacttcactctgactatcagtagcctgcaggctgaagatgtggcagtttattactgtaagcaatcttattctcacagaactttcggcggagggaccaaggtcgagatcaaa

SEQ ID NO: 1161-1209 - skipped (49 entries)

SEQ ID NO: 1210 - protein, 119 aa, synthetic construct
  source 1..119: note = VH ADI-79846
QVQLVQSGAEVKKPGASVKVSCKASGFNIKDYYMHWVRQAPGQRLEWMGWIDLENANTIYDAKFQGRVTITRDTSASTAYMGLSSLRSEDTAVYYCARDHYGRYFYDVWGQGTLVTVSS

SEQ ID NO: 1211 - protein, 26 aa, synthetic construct
  source 1..26: note = ADI-79846
QVQLVQSGAEVKKPGASVKVSCKASG

SEQ ID NO: 1212 - protein, 9 aa, synthetic construct
  source 1..9: note = ADI-79846
FNIKDYYMH

SEQ ID NO: 1213 - protein, 14 aa, synthetic construct
  source 1..14: note = ADI-79846
WVRQAPGQRLEWMG

SEQ ID NO: 1214 - protein, 17 aa, synthetic construct
  source 1..17: note = ADI-79846
WIDLENANTIYDAKFQG

SEQ ID NO: 1215 - protein, 30 aa, synthetic construct
  source 1..30: note = ADI-79846
RVTITRDTSASTAYMGLSSLRSEDTAVYYC

SEQ ID NO: 1216 - protein, 12 aa, synthetic construct
  source 1..12: note = ADI-79846
ARDHYGRYFYDV

SEQ ID NO: 1217 - protein, 11 aa, synthetic construct
  source 1..11: note = ADI-79846
WGQGTLVTVSS

SEQ ID NO: 1218-1219 - skipped (2 entries)

SEQ ID NO: 1220 - protein, 112 aa, synthetic construct; identical to SEQ ID NO: 220
  source 1..112: note = VL ADI-79846

SEQ ID NO: 1221 - protein, 23 aa, synthetic construct
  source 1..23: note = ADI-79846
DIVMTQSPDSLAVSLGERATINC

SEQ ID NO: 1222 - protein, 17 aa, synthetic construct
  source 1..17: note = ADI-79846
KSSQSLLNARTGHNYLA

SEQ ID NO: 1223 - protein, 15 aa, synthetic construct
  source 1..15: note = ADI-79846
WYQQKPGQPPKLLIY

SEQ ID NO: 1224 - protein, 7 aa, synthetic construct
  source 1..7: note = ADI-79846
WASTRES

SEQ ID NO: 1225 - protein, 32 aa, synthetic construct; identical to SEQ ID NO: 25
  source 1..32: note = ADI-79846

SEQ ID NO: 1226 - protein, 8 aa, synthetic construct
  source 1..8: note = ADI-79846
KQSHSHRT

SEQ ID NO: 1227 - protein, 10 aa, synthetic construct
  source 1..10: note = ADI-79846
FGGGTKVEIK

SEQ ID NO: 1228-1249 - skipped (22 entries)

SEQ ID NO: 1250 - DNA, 357 bp, synthetic construct
  source 1..357: mol_type = other DNA; note = VH ADI-79846
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggggctgagcagcctgagatctgaagacacggcggtgtactactgcgcgagagatcattacgggagatatttttacgacgtgtggggccaaggaaccctggtcaccgtctcctca

SEQ ID NO: 1251-1259 - skipped (9 entries)

SEQ ID NO: 1260 - DNA, 336 bp, synthetic construct; identical to SEQ ID NO: 260
  source 1..336: mol_type = other DNA; note = VL ADI-79846

SEQ ID NO: 1261-1309 - skipped (49 entries)

SEQ ID NO: 1310 - protein, 119 aa, synthetic construct; identical to SEQ ID NO: 210
  source 1..119: note = VH ADI-79847

SEQ ID NO: 1311 - protein, 26 aa, synthetic construct
  source 1..26: note = ADI-79847
QVQLVQSGAEVKKPGASVKVSCKASG

SEQ ID NO: 1312 - protein, 9 aa, synthetic construct
  source 1..9: note = ADI-79847
FNIKDYYMH

SEQ ID NO: 1313 - protein, 14 aa, synthetic construct
  source 1..14: note = ADI-79847
WVRQAPGQRLEWMG

SEQ ID NO: 1314 - protein, 17 aa, synthetic construct
  source 1..17: note = ADI-79847
WIDLENANTIYDAKFQG

SEQ ID NO: 1315 - protein, 30 aa, synthetic construct; identical to SEQ ID NO: 115
  source 1..30: note = ADI-79847

SEQ ID NO: 1316 - protein, 12 aa, synthetic construct
  source 1..12: note = ADI-79847
ARDHYHRYFYDV

SEQ ID NO: 1317 - protein, 11 aa, synthetic construct
  source 1..11: note = ADI-79847
WGQGTLVTVSS

SEQ ID NO: 1318-1319 - skipped (2 entries)

SEQ ID NO: 1320 - protein, 112 aa, synthetic construct
  source 1..112: note = VL ADI-79847
DIVMTQSPDSLAVSLGERATINCKSSQSLLNARTGHNYLAWYQQKPGQPPKLLIYWASTRESGVPDRFSGSGSGTDFTLTISSLQAEDVAVYYCKQSHSRRTFGGGTKVEIK

SEQ ID NO: 1321 - protein, 23 aa, synthetic construct
  source 1..23: note = ADI-79847
DIVMTQSPDSLAVSLGERATINC

SEQ ID NO: 1322 - protein, 17 aa, synthetic construct
  source 1..17: note = ADI-79847
KSSQSLLNARTGHNYLA

SEQ ID NO: 1323 - protein, 15 aa, synthetic construct
  source 1..15: note = ADI-79847
WYQQKPGQPPKLLIY

SEQ ID NO: 1324 - protein, 7 aa, synthetic construct
  source 1..7: note = ADI-79847
WASTRES

SEQ ID NO: 1325 - protein, 32 aa, synthetic construct; identical to SEQ ID NO: 25
  source 1..32: note = ADI-79847

SEQ ID NO: 1326 - protein, 8 aa, synthetic construct
  source 1..8: note = ADI-79847
KQSHSRRT

SEQ ID NO: 1327 - protein, 10 aa, synthetic construct
  source 1..10: note = ADI-79847
FGGGTKVEIK

SEQ ID NO: 1328-1349 - skipped (22 entries)

SEQ ID NO: 1350 - DNA, 357 bp, synthetic construct
  source 1..357: mol_type = other DNA; note = VH ADI-79847
caggtgcagctggtgcagtctggggctgaggtgaagaagcctggggcctcagtgaaggtctcctgcaaggcttctggattcaacatcaaggactactatatgcactgggtgcgacaggctcctggacaaaggcttgagtggatgggatggatcgaccttgaaaatgctaacacaatctatgacgctaagtttcagggcagggtcaccataaccagggacacgtccgccagcacagcctacatggagctgagcagcctgagatctgaagacacggcggtgtactactgcgcgagagatcactaccatagatatttttacgacgtgtggggccaaggaaccctggtcaccgtctcctca

SEQ ID NO: 1351-1359 - skipped (9 entries)

SEQ ID NO: 1360 - DNA, 336 bp, synthetic construct
  source 1..336: mol_type = other DNA; note = VL ADI-79847
gacatcgtgatgacccagtctccagactccctggctgtgtctctgggcgagagggccaccatcaactgcaagtccagccagagtcttttaaacgctagaaccggacataactacttagcttggtaccagcagaaaccaggacagcctcctaagctgctcatttactgggcatctacccgggaatccggggtccctgaccgattcagtggcagcgggtctgggacagacttcactctcactatcagtagcctgcaggctgaagatgtggcagtttattactgtaagcaatctcattctcgtagaactttcggcggagggaccaaggttgagatcaaa

(19) United States
(12) Patent Application Publication
(10) Pub. No.: US 2026/0152557 A1
(43) Pub. Date: Jun. 4, 2026

(54) PH-DEPENDENT ANTI-CD3 ANTIBODIES AND METHODS RELATING THERETO

(71) Applicant: ADIMAB, LLC, Lebanon, NH (US)

(72) Inventors: Michael Benjamin BATTLES, Lebanon, NH (US); Paul WIDBOOM, Lebanon, NH (US)

(21) Appl. No.: 19/122,279

(22) PCT Filed: Oct. 18, 2023
(86) PCT No.: PCT/US2023/077148

§ 371 Date: Apr. 18, 2025

Related U.S. Application Data
(60) Provisional application No. 63/417,118, filed on Oct. 18, 2022.

(51) Int. Cl. C07K 16/28; A61K 31/7088; A61K 35/00; A61K 38/00; C12N 15/11; C12N 15/63

(52) U.S. Cl. CPC C07K 16/2809; A61K 31/7088; A61K 35/00; A61K 38/00; C12N 15/11; C12N 15/63; C07K 2317/14; C07K 2317/31; C07K 2317/522; C07K 2317/526; C07K 2317/565; C07K 2317/73; C07K 2317/92

(57) ABSTRACT

The present disclosure provides anti-CD3 antibodies and antigen-binding fragments and multispecific antibodies and antibody fragments thereof. The present disclosure further provides nucleic acids and vectors encoding such antibodies or antibody fragments and cells comprising such nucleic acids. Further provided are pharmaceutical compositions, in vivo methods, screening methods, and manufacturing methods relating to anti-CD3 antibodies and antigen-binding fragments.

### CROSS-REFERENCE TO RELATED APPLICATIONS

This application claims priority to U.S. Provisional Application No. 63/417,118, filed on Oct. 18, 2022, entitled “PH-DEPENDENT ANTI-CD3 ANTIBODIES AND METHODS RELATING THERETO”, the contents of which are incorporated by reference in their entirety herein.

### REFERENCE TO AN ELECTRONIC SEQUENCE LISTING

The contents of the electronic sequence listing (1160430o003813.xml; Size: 508,346 bytes; and Date of Creation: Oct. 11, 2023) is herein incorporated by reference in its entirety.

### BACKGROUND

Cell proliferative disorders, such as cancer, are characterized by the uncontrolled growth of cell subpopulations. They are the leading cause of death in the developed world and the second leading cause of death in developing countries, with a total number of new cancer cases per year expected to rise to 23.6 million by 2030. The National Cancer Institute estimates that almost 2 million new cases of cancer will be diagnosed in the U.S. and greater than 600,000 Americans will die of cancer in 2020. Cancer care thus represents a significant and ever-increasing societal burden.

The idea of using the cytotoxic capacity of T cells to kill tumor cells through use of CD3 targeting bispecific antibodies dates back to the mid-1980s. (Staerz et al. Nature 1985 314: 628-32). Many bispecific antibodies developed to date contain a first binding site specific to CD3 for T-cell recruitment and activation, and a second binding site for a targeted disease-associated antigen, such as an antigen produced by a tumor cell. CD3 bispecific antibodies bind to both the CD3 surface receptor on T cells and their second target antigen such as a protein expressed on tumors such that available T cells can bind to target-expressing cells via bridging by the CD3 bispecific antibody, irrespective of the peptide/MUC specificity of their T-cell receptor. See, e.g., Bassan, 2012, Blood 120:5094-95). Bridging of T cells and tumor cells using CD3 bispecific antibodies can induce dramatic regression of advanced-stage malignancies and, in some cases, lead to complete remission. Currently, more than 25 different CD3 bispecific antibodies are in clinical development for treatment of hematologic malignancies or solid cancers by targeting CD19, CD20, CD33, and CD123, or EpCAM, HER2, PSMA, and CEA, respectively. (See, e.g., Liu et al. Front Immunol 2017 8:38).

A variety of anti-CD3 antibodies are known in the art, including monoclonal and bispecific antibody formats. See, e.g., U.S. Pat. Nos. 7,262,276; 7,635,472; 7,862,813; 9,587,021; and 10,174,124. However, many of these anti-CD3 antibodies elicit excess production of cytokines, often leading to toxic cytokine release syndrome (CRS). Because the anti-CD3 binding region of the bispecific antibody engages all T cells, the high cytokine-producing CD4 T cell subset is recruited. Accordingly, there is an unmet need for the provision for anti-CD3 antibodies that display desirable CRS risk profiles and are safe and efficacious in, for example, binding specifically to CD3 expressed on T-cels, activating T-cells and (re)-directing the activated T-cells to kill target cells, and doing this with diminished risk of eliciting CRS.

One approach to developing CD3 binding regions that display desirable CRS risk profiles, is to engineer CD3 binding regions with pH-dependent antigen binding. Incorporation of histidines and/or other ionizable residues into the binding interfaces of antibodies and other proteins has previously been used to engineer pH-dependent antigen-binding (see, e.g., Igawa et al., Nature Biotechnology 28:1203-1207 (2010)). Protonation of histidine side chains in binding interfaces may alter electrostatic interactions and/or induce conformational changes that lead to pH-dependent differences in binding affinity (Gera et al., PLOS ONE 7(11) e48928. doi:10.1371/2012). Recognizing that the pH range of human blood is about 7.6-7.8, whereas tumor cells have an extracellular pH of about 6.3-6.5 due at least in part to accumulation of metabolic acids that are inefficiently cleared because of poor tumor vascularization, Applicant previously engineered pH-dependent CD3 binding antibodies with preferential CD3 binding at low(er) pH values, which promote binding and activity in and around the tumor microenvironment (WO2020247932A1). Without being bound by theory, it is believed that CD3 binding regions engineered to preferably bind to CD3 at a lower pH, e.g., pH ˜6, may result in selective and sustained cytotoxic activity at or around the tumor site, thereby potentially reducing or eliminating on-target off-tumor effects as well as improving half-life and enabling reduced dosing.

### SUMMARY

The present disclosure relates to: improved pH4-dependent anti-CD3 antibodies and antigen-binding fragments thereof, e.g., those that bind to not only human but also cynomolgus CD3 and/or provide improved binding to CD3 and/or CD3-expressing cells at pH 6.0; and methods of using or producing the same.

In one aspect, the disclosure provides pH-dependent anti-CD3 antibodies and antigen-binding fragments.

In some embodiments, the anti-CD3 antibody or antigen-binding fragment thereof may comprise: (A) a heavy chain variable domain (VH) polypeptide comprising: (a) a VH complementarity determining region 1 (CDR-H1) comprising the amino acid sequence of: (i) the CDR-H1 contained in ADI-74968, ADI-74967, ADI-74966, ADI-74965, ADI-79842, ADI-79843, ADI-79848, ADI-79844, ADI-79845, ADI-79846, or ADI-79847; and/or (ii) FNIKDYYMH (SEQ ID NO: 12, 612, 512, 412, 312, 712, 812, 912, 1012, 1112, 1212, or 1312); (b) a VH complementarity determining region 2 (CDR-H2) comprising the amino acid sequence of: (i) the CDR-H2 contained in ADI-74968, ADI-74967, ADI-74966, ADI-74965, ADI-79842, ADI-79843, ADI-79848, ADI-79844, ADI-79845, ADI-79846, or ADI-79847; and/or (it) WIDLENANTIYDAKFQG (SEQ ID NO: 14, 614, 514, 414, 314, 714, 814, 914, 1014, 1114, 1214, or 1314); and/or (c) a VH complementarity determining region 3 (CDR-H3) comprising the amino acid sequence of: (i) the CDR-H3 contained in ADI-74968, ADI-74967, ADI-74966, ADI-74965, ADI-79842, ADI-79843, ADI-79848, ADI-79844, ADI-79845, ADI-79846, or ADI-79847; (ii) ARDX₂Y X₃RYFYDV (SEQ ID NO: 16), wherein X₂ is A or H and X₃ is H or G; and/or (iii) ARDAYHRYFYDV (SEQ ID NO: 616, 316, or 816), ARDHYHRYFYDV (SEQ ID NO: 516, 416, or 1316), ARDHYGRYFYDV (SEQ ID NO: 716 or 1216), or ARDAYGRYFYDV (SEQ ID NO: 916, 1016, or 1116); and/or (B) a light chain variable domain (VL) polypeptide comprising: (a) a VL complementarity determining region 1 (CDR-L1) comprising the amino acid sequence of: (i) the CDR-L1 contained in ADI-74968, ADI-74967, ADI-74966, ADI-74965, ADI-79842, ADI-79843, ADI-79848, ADI-79844, ADI-79845, ADI-79846, or ADI-79847; (ii) KSSQSLLNARTGX₅NYLA (SEQ ID NO: 22), wherein X₅ is H or K; and/or (iii) KSSQSLLNARIGHNYLA (SEQ ID NO: 622, 422, 322, 922, 1222, or 1322) or KSSQSLLNARTGKNYLA (SEQ ID NO: 522, 722, 822, 1022, or 1122); (b) a VL complementarity determining region 2 (CDR-L2) comprising the amino acid sequence of: (i) the CDR-L2 contained in ADI-74968, ADI-74967, ADI-74966, ADI-74965, ADI-79842, ADI-79843, ADI-79848, ADI-79844, ADI-79845, ADI-79846, or ADI-79847; and/or (ii) WASTRES (SEQ ID NO: 24, 624, 524, 424, 324, 724, 824, 924, 1024, 1124, 1224, or 1324); and/or (c) a VL complementarity determining region 3 (CDR-L3) comprising the amino acid sequence of (i) the CDR-L3 contained in ADI-74968, ADI-74967, ADI-74966, ADI-74965, ADI-79842, ADI-79843, ADI-79848, ADI-79844, ADI-79845, ADI-79846, or ADI-79847; (ii) KQSX₆SX₇RT (SEQ ID NO: 26), wherein X₆ is Y or H and X₇ is H or R; and/or (iii) KQSYSHRT (SEQ ID NO: 626, 426, or 1126), KQSHSHRT (SEQ ID NO: 526, 326, or 1226), KQSHSRRT (SEQ ID NO: 1026 or 1326), or KQSYSRRT (SEQ ID NO: 726, 826, or 926), or the anti-CD3 antibody or antigen-binding fragment may comprise a combination of one or more of the foregoing CDRs.

In some cases, the anti-CD3 antibody and/or antigen-binding fragment does not comprise: (i) at least one of the CDR-H1, CDR-H2, CDR-H3, CDR-L1, CDR-L2, and CDR-L3 contained in ADI-48587; or (ii) at least one of a CDR-H1 comprising FNIKDYYMH (SEQ ID NO: 12 or 212), a CDR-H2 comprising WIDLEENANTIYDAKFQOG (SEQ ID NO: 14 or 214), a CDR-H3 ARDHYHRYFYDV (SEQ ID NO: 216), a CDR-L1 comprising KSSQSLLNARTGHNYLA (SEQ ID NO: 222), a CDR-H2 comprising WASTRES (SEQ ID NO 24 or 224), and a CDR-L3 comprising KQSHSHRT (SEQ ID NO: 226).

In certain cases, the anti-CD3 antibody and/or antigen-binding fragment does not comprise: (i) at least one of the CDR-H3, CDR-L1, and CDR-L3 contained in ADI-48587; or (ii) at least one of a CDR-H3 comprising ARDHYHRYFYDV (SEQ ID NO: 216), a CDR-L1 comprising KSSQSLLNARTGHNYLA (SEQ ID NO: 222), and a CDR-L3 comprising KQSHSHRT (SEQ ID NO: 226).

In some cases, the anti-CD3 antibody and/or antigen-binding fragment does not comprise: (i) at least one of the CDR-H1, CDR-H2, CDR-H3, CDR-L1, CDR-L2, and CDR-L3 contained in ADI-26906; or (ii) at least one of a CDR-H1 comprising FNIKDYYMH (SEQ ID NO: 12 or 112), a CDR-H2 comprising WIDLENANTIYDAKFQG (SEQ ID NO: 14 or 114), a CDR-H3 comprising ARDAYGRYFYDV (SEQ ID NO: 116), a CDR-L1 comprising KSSQSLLNARTGKNYLA (SEQ ID NO: 122), a CDR-L2 comprising WASTRES (SEQ ID NO: 24 or 124), and a CDR-L3 comprising KQSYSRRT (SEQ ID NO: 126).

In certain cases, the anti-CD3 antibody and/or antigen-binding fragment does not comprise: (i) the CDR-H3, CDR-L1, and CDR-L3 contained in ADI-26906; or (ii) a CDR-H3 comprising ARDAYGRYFYDV (SEQ ID NO: 116), a CDR-L1 comprising KSSQSLLNARTGKNYLA (SEQ ID NO: 122), and a CDR-L3 comprising KQSYSRRT (SEQ ID NO: 126).

In certain embodiments, the anti-CD3 antibody or antigen-binding fragment may comprise: (A) a VH polypeptide comprising: (a) a CDR-H) comprising the amino acid sequence of: (i) the CDR-H1 contained in ADI-74968, ADI-74967, ADI-74966, or ADI-74965; and/or (ii) FNIKDYYMH (SEQ ID NO: 12, 612, 512, 412, or 312); (b) a CDR-H2 comprising the amino acid sequence of: (i) the CDR-H2 contained in ADI-74968, ADI-74967, ADI-74966, or ADI-74965; and/or (ii) WIDLENANTIYDAKFQG (SEQ ID NO: 14, 614, 514, 414, or 314); and/or (c) a CDR-H3 comprising the amino acid sequence of: (i) the CDR-H3 contained in ADI-74968, ADI-74967, ADI-74966, or ADI-74965; (ii) ARDX₂Y X₃RYFYDV (SEQ ID NO: 16), wherein X₂ is A or H and X₃ is H; and/or (iii) ARDAYHRYFYDV (SEQ ID NO: 616 or 316) or ARDHYHRYFYDV (SEQ ID NO: 516 or 416); and/or (B) a VL polypeptide comprising: (a) a CDR-L1 comprising the amino acid sequence of (i) the CDR-L1 contained in ADI-74968, ADI-74967, ADI-74966, or ADI-74965; (ii) KSSQSLLNARTGX₅NYLA (SEQ ID NO: 22), wherein X₅ is H or K; and/or (iii) KSSQSLLNARTGHNYLA (SEQ ID NO: 622, 422, or 322) or KSSQSLLNARTGKNYLA (SEQ ID NO: 522); (b) a CDR-L2 comprising the amino acid sequence of: (i) the CDR-L2 contained in ADI-74968, ADI-74967, ADI-74966, or ADI-74965; and/or (ii) WASTRES (SEQ ID NO: 24, 624, 524, 424, or 324); and/or (c) a CDR-L3 comprising the amino acid sequence of: (i) the CDR-L3 contained in ADI-74968, ADI-74967, ADI-74966, or ADI-74965; (ii) KQSX₆SX₇RT (SEQ ID NO: 26), wherein X₆ is Y or H and X is H; and/or (iii) KQSYSHRT (SEQ ID NO: 626 or 426) or KQSHSHRT (SEQ ID NO: 526 or 326).

In particular embodiments, the anti-CD3 antibody or antigen-binding fragment may comprise: (A) a V H polypeptide comprising: (a) a CDR-H comprising the amino acid sequence of: (i) the CDR-H1 contained in ADI-74968; and/or (ii) FNIKDYYMH (SEQ ID NO: 12 or 612); (b) a CDR-H2 comprising the amino acid sequence of: (i) the CDR-H2 contained in ADI-74968; and/or (ii) WIDLENANTIYDAKFQG (SEQ ID NO: 14 or 614); and/or (c) a CDR-H3 comprising the amino acid sequence of: (i) the CDR-H3 contained in ADI-74968; and/or (ii) ARDAYHRYFYDV (SEQ ID NO: 616); and/or (B) a VL polypeptide comprising: (a) a CDR-L1 comprising the amino acid sequence of: (i) the CDR-L1 contained in ADI-74968; (ii) KSSQSLLNARTGHNYLA (SEQ ID NO: 622); (b) a CDR-L2 comprising the amino acid sequence of: (i) the CDR-L2 contained in ADI-74968; and/or (ii) WASTRES (SEQ ID NO: 24 or 624); and/or (c) a CDR-L3 comprising the amino acid sequence of: (i) the CDR-L3 contained in ADI-74968; (ii) KQSYSHRT (SEQ ID NO: 626).

In particular embodiments, the anti-CD3 antibody or antigen-binding fragment may comprise: (A) a VH polypeptide comprising: (a) a CDR-H1 comprising the amino acid sequence of: (i) the CRR-H1 contained in ADI-74967; and/or (ii) FNIKDYYMH (SEQ ID NO: 12 or 512); (b) a CDR-H2 comprising the amino acid sequence of: (i) the CDR-H2 contained in ADI-74967; ad/or (ii) WIDLENANTIYDAKFQG (SEQ ID NO: 14 or 514); and/or (c) a CDR-H3 comprising the amino acid sequence of: (i) the CDR-H3 contained in ADI-74967; (ii) ARDHYHRYFYDV (SEQ ID NO: 516); and/or (B) a VL polypeptide comprising: (a) a CDR-L1 comprising the amino acid sequence of: (i) the CDR-L1 contained in ADI-74967; (ii) KSSQSLLNARTGKNYLA (SEQ ID NO: 522); (b) a CDR-L2 comprising the amino acid sequence of: (i) the CDR-L2 contained in ADI-74967; and/or (ii) WASTRES (SEQ ID NO 24, 624, 524); and/or (c) a CDR-L3 comprising the amino acid sequence of: (i) the CDR-L3 contained in ADI-74967; (ii) KQSHSHRT (SEQ ID NO: 526).

In some cases, the anti-CD3 antibody or antigen-binding fragment according to any of the above embodiments may comprise: (A) a VH polypeptide comprising said CDR-H1, said CDR-H2, and said CDR-H13; and/or (B) a VL polypeptide comprising said CDR-L1, said CDR-L2, and said CDR-L3.

In certain cases, the ani-CD3 antibody or antigen-binding fragment according to any of the above embodiments may comprise: (A) a VH polypeptide comprising said CDR-H1, said CDR-H2, and said CDR-H3; and (B) a VL polypeptide comprising said CDR-L1, said CDR-L2, and said CDR-L3.

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-74968; or (ii) a CDR-H1 comprising FNIKDYYMH (SEQ ID NO: 12 or 612), a CDR-H2 comprising WIDLENANTIYDAKFQG (SEQ ID NO: 14 or 614), a CDR-H3 comprising ARDAYHRYFYDV (SEQ ID NO: 616), a CDR-L1 comprising KSSQSLLNARTGHNYLA (SEQ ID NO: 622); a CDR-L2 comprising WASTRES (SEQ ID NO: 24 or 624), and a CDR-L3 comprising KQSYSHRT (SEQ ID NO: 626).

In particular embodiments, the ani-CD3 antibody or antigen-binding fragment may comprise: (i) the CDR-H1, CDR-H2, CDR-H3, CDR-L1, CDR-L2, and the CDR-L3 contained in ADI-74967; or (ii) a CDR-H1 comprising FNIKDYYMH (SEQ ID NO: 12 or 512), a CDR-H2 comprising WIDLENANTIYDAKFQG (SEQ ID NO: 14 or 514), a CDR-H3 comprising ARDHYHRYFYDV (SEQ ID NO: 516), a CDR-L1 comprising KSSQSLLNARTGKNYLA (SEQ ID NO: 522), a CDR-L2 comprising WASTRES (SEQ TID NO: 24 or 524), and a CDR-L3 comprising KQSHSHRT (SEQ ID NO: 526).

In particular embodiments, the anti-CD3 antibody or antigen-binding fragment may comprise: (i) the CDR-H1, CDR-H2, CDR-H3, CDR-L1, CDR-L2, and the CDR-L3 contained m ADI-74966; or (ii) a CDR-H1 comprising FNIKDYYMH (SEQ ID NO: 12 or 412), a CDR-H2 comprising WIDLENANTIYDAKFQG (SEQ ID NO: 14 or 414), a CDR-H3 comprising ARDHYHRYFYDV (SEQ ID NO: 416), a CDR-L1 comprising KSSQSLLNARTGHNYLA (SEQ ID NO: 422), a CDR-L2 comprising WASTRES (SEQ ID NO: 24 or 424), and a CDR-L3 comprising KQSYSHRT (SEQ ID NO: 426).

In particular embodiments, the anti-CD3 antibody or antigen-binding fragment may comprise: (i) the CDR-H1 CDR-H2, CDR-H3, CDR-L1, CDR-L2, and the CDR-L3 contained in ADI-74965; or (ii) a CDR-H1 comprising FNIKDYYMH (SEQ ID NO: 12 or 312), a CDR-H2 comprising WIDLENANTIYDAKFQG (SEQ ID NO: 14 or 314), a (CDR-H3 comprising ARDAYHRYFYDV (SEQ ID NO: 316), a CDR-L1 comprising KSSQSLLNARTGHNYLA (SEQ TID NO: 322), a CDR-L2 comprising WASTRES (SEQ ID NO: 24 or 324), and a CDR-L3 comprising KQSHSHRT (SEQ ID NO: 326).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79842; or (i) a CDR-H1 comprising FNIKDYYMH (SEQ ID NO: 12 or 712), a CDR-H2 comprising WIDLENANTIYDAKFQG (SEQ ID NO: 14 or 714), a CDR-H3 comprising ARDHYGRYFYDV (SEQ ID NO: 716), a CDR-L1 comprising KSSQSLLNARTGKNYLA (SEQ ID NO: 722), a CDR-L2 comprising WASTRES (SEQ ID NO: 24 or 724), and a CDR-L3 comprising KQSYSRRT (SEQ ID NO: 726).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79843; or (ii) a CDR-H1 comprising FNIKDYYMH (SEQ ID NO: 12 or 812), a CDR-F12 comprising WIDLENANTIYDAKFQG (SEQ ID NO: 14 or 814), a CDR-H3 comprising ARDAYHRYFYDV (SEQ ID NO: 816), a CDR-L1 comprising KSSQSLLNARTGKNYLA (SEQ ID NO: 822), a CDR-L2 comprising WASTRES (SEQ ID NO: 24 or 824), and a CDR-L3 comprising KQSYSRRT (SEQ ID NO: 826).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79848; or (ii) a CDR-H1 comprising FNIKDYYMH (SEQ ID NO: 12 or 912), a CDR-H2 comprising WIDLENANTIYDAKFQG (SEQ ID NO: 14 or 914), a CDR-H3 comprising ARDAYGRYFYDV (SEQ ID NO: 916), a CDR-L1 comprising KSSQSLLNARTGHNYLA (SEQ TID NO: 922), a CDR-L2 comprising WASTRES (SEQ ID NO: 24 or 924), and a CDR-L3 comprising KQSYSRRT (SEQ TID NO: 926).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79844; or (ii) a CDR-H1 comprising FNIKDYYMH (SEQ ID NO: 12 or 1012), a CDR-H2 comprising WIDLENANTIYDAKFQG (SEQ ID NO: 14 or 1014), a CDR-H3 comprising ARDAYGRYFYDV (SEQ ID NO: 1016), a CDR-L1 comprising KSSQSLLNARTGKNYLA (SEQ ID NO: 1022), a CDR-L2 comprising WASTRES (SEQ ID NO: 24 or 1024), and a CDR-L3 comprising KQSHSRRT (SEQ ID NO: 1026).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79845; or (ii) a CDR-H1 comprising FNIKDYYMH (SEQ ID NO: 12 or 1112), a CDR-H2 comprising WIDLENANTIYDAKFQG (SEQ ID NO: 14 or 1114), a CDR-H3 comprising ARDAYGRYFYDV (SEQ ID NO: 1116), a CDR-L1 comprising KSSQSLLNARTGKNYLA (SEQ ID NO: 1122), a CDR-L2 comprising WASTRES (SEQ ID NO: 24 or 1124), and a CDR-L3 comprising KQSYSHRT (SEQ ID NO: 1126).

In particular embodiments, the ani-CD3 antibody or antigen-binding fragment may comprise: (i) the CDR-H1, CDR-H2, CDR-H3, CDR-L1, CDR-L2, and the CDR-L3 contained in ADI-79846; or (it) a CDR-H1 comprising FNIKDYYMH (SEQ ID NO: 12 or 1212), a CDR-H2 comprising WIDLENANTIYDAKFQG (SEQ ID NO: 14 or 1214), a CDR-H3 comprising ARDAYGRYFYDV (SEQ ID NO: 1216), a CDR-L1 comprising KSSQSLLNARTGKNYLA (SEQ ID NO: 1222), a CDR-L2 comprising WASTRES (SEQ ID NO: 24 or 1224), and a CDR-L3 comprising KQSHSHRT (SEQ ID NO: 1226).

In particular embodiments, the anti-CD3 antibody or antigen-binding fragment may comprise: (i) the CDR-H1, CDR-H2, CDR-H3, CDR-L1, CDR-L2, and the CDR-L3 contained in ADI-79846; or (ii) a CDR-H1 comprising FNIKDYYMH (SEQ ID NO: 12 or 1312), a CDR-H2 comprising WIDLENANTIYDAKFQG (SEQ ID NO: 14 or 1314), a CDR-H3 comprising ARDHYGRYFYDV (SEQ ID NO: 1316), a CDR-L1 comprising KSSQSLLNARTGHNYLA (SEQ ID NO: 1322), a CDR-L2 comprising WASTRES (SEQ ID NO: 24 or 1324), and a CDR-L3 comprising KQSHSRRT (SEQ ID NO: 1326).

In some cases, in the anti-CD3 antibody or Antigen-binding fragment according to any of the above embodiments: (A) the VH polypeptide comprises: (a) a VH framework region 1 (FR-H1) comprising the amino acid sequence of: (i) the FR-H1 contained in ADI-74968, ADI-74967, ADI-74966, ADI-74965, ADI-79842, ADI-79843, ADI-79848, ADI-79844, AD-79845, ADI-79846, or ADI-79847; and/or (ii) QVQLVQSGAEVKKPGASVKVSCKASG (SEQ ID NO: 11, 611, 511, 411, 311, 711, 811, 911, 1011, 1111, 1211, or 1311); (b) a VH framework region 2 (FR-H2) comprising the amino acid sequence of: (i) the FR-H2 contained in ADI-74968, ADI-74967, ADI-74966, ADI-74965, ADI-79842, ADI-79843, ADI-79848, ADI-79844, ADI-79845, ADI-79846, or ADI-79847; and/or (ii) WVRQAPGQRLEWMG (SEQ ID NO: 13, 613, 513, 413, 313, 713, 813, 913, 1013, 1113, 1213, or 1313); (c) a VH framework region 3 (FR-H3) comprising the amino acid sequence of: (i) the FR-H3 contained in ADI-74968, ADI-74967, ADI-74966, ADI-74965, ADI-79842, ADI-79843, ADI-79848, ADI-79844, ADI-79845, ADI-79846, or ADI-79847; (ii) RVTITRDTSASTAYMX₁LSSLRSEDTAVYYC (SEQ ID NO: 15), wherein X₁ is E or G; and/or (iii) RVTITRDTSASTAYMELSSLRSEDTAVYYC (SEQ ID NO: 615, 515, 415, 315, 715, 815, 915, 1015, 1115, or 1315) or RVTITRDTSASTAYMGLSSLRSEDTAVYYC (SEQ ID NO: 1215); and/or (d) a VH framework region 4 (FR-H4) comprising the amino acid sequence of: (i) the FR-H4 contained in ADI-74968, ADI-74967, ADI-74966, ADI-74965, ADI-79842, ADI-79843, ADI-79848, ADI-79844, ADI-79845, ADI-79846, or ADI-79847; and/or (ii) WGQGTLVTVSS (SEQ ID NO: 17, 617, 517, 417, 317, 717, 817, 917, 1017, 1117, 1217, or 1317); and/or (B) the VL polypeptide comprises: (a) a VL framework region 1 (FR-L1) comprising the amino acid sequence of: (i) the FR-L1 contained in ADI-74968, ADI-74967, ADI-74966, ADI-74965, ADI-79842, ADI-79843, ADI-79848, ADI-79844, ADI-79845, ADI-79846, or ADI-79847; (ii) X₄IVMTQSPDSLAVSLGERATINC (SEQ ID NO: 21), wherein X₁ is D or G; and/or (iii) DIVMTQSPDSLAVSLGERATINC (SEQ ID NO: 621, 421, 321, 721, 821, 921, 1021, 1121, 1221, or 1321) or GIVMTQSPDSLAVSLGERATINC (SEQ ID NO: 521); (b) a VL framework region 2 (FR-L2) comprising the amino acid sequence of: (i) the FR-L2 contained in ADI-74968, ADI-74067, ADI-74966, ADI-74965, ADI-79842, ADI-79843, ADI-79848, ADI-79844, ADI-79845, ADI-79846, or ADI-79847; and/or (ii) WYQQKPGQPPKLLIY (SEQ ID NO: 23, 623, 523, 423, 323, 723, 823, 923, 1023, 1123, 1223, or 1323); (c) a VL framework region 3 (FR-L3) comprising the amino acid sequence of (i) the FR-L3 contained in ADI-74968, ADI-74967, ADI-74966, ADI-74965, ADI-79842, ADI-79843, ADI-79848, ADI-79844, ADI-79845, ADI-79846, or ADI-79847; and/or (ii) GVPDRFSGSGSGTDFTLTISSLQAEDVAVYYC (SEQ ID NO: 25, 625, 525, 425, 325, 725, 825, 925, 1025, 1125, 1225, or 1325); and/or (d) a VL framework region 4 (FR-L4A) comprising the amino acid sequence of: (i) the FR-L4 contained in ADI-74968, ADI-74967, ADI-74966, ADI-74965, ADI-79842, ADI-79843, ADI-79848, ADI-79844, ADI-79845, ADI-79846, or ADI-79847; and/or (ii) FGGGTKVEIK (SEQ ID NO: 27, 627, 527, 427, 327, 727, 827, 927, 1027, 1127, 1227, or 1327), or the anti-CD3 antibody or antigen-binding fragment may comprise a VH and/or a VL that comprises any combination of the foregoing VH and VL framework regions.

In certain cases, the anti-CD3 antibody or antigen-binding fragment according to any of the above embodiments may comprise: (i) the FR-H1, FR-H2, FR-H3, FL-H4, FR-L1, FR-L2, FR-L3, and the FR-L4 contained in ADI-74968, ADI-74966, ADI-74965, ADI-79842, ADI-79843, ADI-79848, ADI-79844, ADI-79845, or ADI-79847; or (ii) a FR-H3 comprising QVQLVQSGAEVKKPGASVKVSCKASG (SEQ ID NO: 11, 611, 411, 311, 711, 811, 911, 1011, 1111, or 1311), a FR-H2 comprising WVRQAPGQRLEWMG (SEQ ID NO: 13, 613, 413, 313, 713, 813, 913, 1013, 11113, or 1313), a FR-H3 comprising RVTITRDTSASTAYMELSSLRSEDTAVYYC (SEQ ID NO: 615, 415, 315, 715-815, 915, 1015, 1115, or 1315), a FR-H4 comprising WGQGTLVTVSS (SEQ ID NO: 17, 617, 417, 317, 717, 817, 917, 1017, 1117, or 1317), a FR-L1 comprising DIVMTQSPDSLAVSLGERATINC (SEQ ID NO: 621, 421, 321, 721, 821, 921, 1021, 1121, or 1321), a FR-L2 comprising WYQQKPGQPPKLLIY (SEQ ID NO: 23, 623, 423, 323, 723, 823, 923, 1023, 1123, or 1323), a FR-L3 comprising GVPDRFSGSGSGTDFTLTISSLQAEDVAVYYC (SEQ ID NO: 25, 625, 425, 325, 725, 825, 925, 1025, 1125, or 1325), and a FR-L4 comprising FGGGTKVEIK (SEQ ID NO: 27, 627, 427, 327, 727, 827, 927, 1027, 1127, or 1327).

In certain cases, the anti-CD3 antibody or antigen-binding fragment according to any of the above embodiments may comprise: (i) the FR-H1, FR-H2, FR-H3, FL-H4, FR-L1, FR-L2, FR-L3, and the FR-L4 contained in ADI-74967; or (ii) a FR-H1 comprising QVQLVQSGAEVKKPGASVKVSCKASG (SEQ ID NO: 11 or 511), a FR-H2 comprising WVRQAPGQRLEWMG (SEQ ID NO: 13 or 513), a FR-H3 comprising RVTITRDTSASTAYMELSSLRSEDTAVYYC (SEQ ID NO: 515), a FR-H4 comprising WGQGTLVTVSS (SEQ ID NO: 17 or 517), a FR-L1 comprising GIVMTQSPDSLAVSLGERATINC (SEQ ID NO: 521), a FR-L2 comprising WYQQKPGQPPKLLIY (SEQ ID NO: 23 or 523), a FR-L3 comprising GVPDRFSGSGSGTDFTLTISSLQAEDVAVYYC (SEQ ID NO: 25 or 525), and a FR-LA comprising FGGGTKVEIK (SEQ ID NO: 27 or 527).

In certain cases, the anti-CD3 antibody or antigen-binding fragment according to any of the above embodiments may comprise: (i) the FR-H1, FR-H2, FR-H3, FR-H4, FR-L1, FR-L2, FR-L3, and the FR-L4 contained in ADI-74968, ADI-74966, ADI-74965, ADI-79842, ADI-79843, ADI-79848, ADI-79844, ADI-79845, or ADI-79847; or (ii) a FR-H1 comprising QVQLVQSGAEVKKPGASVKVSCKASG (SEQ ID NO: 11 or 1211), a FR-H2 comprising WVRQAPGQRLEWMG (SEQ ID NO: 13 or 1213), a FR-H3 comprising RVTITRDTSASTAYMGLSSLRSEDTAVYYC (SEQ ID NO: 1215), a FR-H4 comprising WGQGTLVTVSS (SEQ ID NO: 17 or 1217), a FR-L1 comprising DIVMTQSPDSLAVSLGERATINC (SEQ ID NO: 1221), a FR-L2 comprising WYQQKPGQPPKLLIY (SEQ ID NO: 1223), a FR-L3 comprising GVPDRFSGSGSGTDFTLTISSLQAEDVAVYYC (SEQ ID NO: 1225), and a FR-LA comprising FGGGTKVEIK (SEQ ID NO: 1227).

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-7496; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 611, 612, 613, 614, 615, 616, 617, 621, 622, 623, 624, 625, 626, and 627, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-H1, CDR-L1, FR-L2, CDR-L2, FR-L3, CDR-L3, and the FR-L4 contained in ADI-74967, or (hi) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 511, 512, 513, 514, 515, 516, 517, 521, 522, 523, 524, 525, 526, and 527, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74966; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-LA comprising SEQ ID NOS: 411, 412, 413, 414, 415, 416, 417, 421, 422, 423, 424, 425, 426, and 427, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74965; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 311, 312, 313, 314, 315, 316, 317, 321, 322, 323, 324, 325, 326, and 327, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79842; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 711, 712, 713, 714, 715, 716, 717, 721, 722, 723, 724, 725, 726, and 727, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79843; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 811, 812, 813, 814, 815, 816, 817, 821, 822, 823, 824, 825, 826, and 827, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79848; or (ii) a FR-H1, CDR-H1, FR-H2, CDR-H2, FR-H3, CDR-H3, FR-H4, FR-L1, CDR-L, FR-L2, CDR-L2, FR-L3, CDR-L3, and FR-L4 comprising SEQ ID NOS: 911, 912, 913, 914, 915, 916, 917, 921, 922, 923, 924, 925, 926, and 927, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79844; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 1011, 1012, 1013, 1014, 1015, 1016, 1017, 1021, 1022, 1023, 1024, 1025, 1026, and 1027, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79845; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-4 comprising SEQ ID NOS: 1111, 1112, 1113, 1114, 1115, 1116, 1117, 1121, 1122, 1123, 1124, 1125, 1126, and 1127, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79846; or (ii) a FR-H1, CDR-H1, FR-12, CDR-H2, FR-H3, CDR-H3, FR-H4, FR-H1, CDR-L1, FR-L2, CDR-L2, FR-L3, CDR-L3, and FR-L4 comprising SEQ ID NOS: 1211, 1211, 1213, 1214, 1215, 1216, 1217, 1221, 1222, 1223, 1224, 1225, 1226, and 1227, respectively.

In particular embodiments, the anti-CD3 antibody or antigen-binding fragment may comprise: (i) the FR-H1, CDR-H1, FR-H2, CDR-H2, FR-H3, CDR-H3, FR-H4, FR-L1, CDR-L1, FR-2, CDR-L2, FR-L3, CDR-L3, and the FR-L4 contained in ADI-79847; or (ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 1311, 1312, 1313, 1314, 1315, 1316, 1317, 1321, 1322, 1323, 1324, 1325, 1326, and 1327, respectively.

In certain embodiments, the anti-CD3 antibody or antigen-binding fragment may comprise: (A) a VH polypeptide comprising an amino acid sequence at least 80%, at least 85%, at least 90%, at least 92%, at least 94%, at least 95%, at least 96%, at least 97%, at least 98%, at least 99%, or 100% identical to SEQ ID NO: 610, 510, 410, 310, 710, 810, 910, 1010, 1110, 1210, or 1310; and/or (B) a VL polypeptide comprising the amino acid sequence at least 80%, at least 85%, at least 90%4), at least 92%, at least 94%, at least 95%, at least 96%, at least 97%, at least 98%, at least 99%, or 100% identical to SEQ ID NO: 620, 520, 420, 320, 720, 820, 920, 1020, 1120, 1220 or 1320.

In particular embodiments, the anti-CD3 antibody or antigen-binding fragment may comprise: (A) a VH polypeptide comprising (i) the CDR-H1, the CDR-H2, and the CFR-H3 sequences of SEQ ID NOS: 612, 614, and 616, respectively and (ii) an amino acid sequence at least 90% identical to SEQ ID NO: 610; and (B) a VL polypeptide comprising (i) the CDR-L1, the CDR-L2, and the CFR-L3 sequences of SEQ ID NOS: 622, 624, and 626, respectively; and (ii) an amino acid sequence at least 90% identical to SEQ ID NO: 620.

In particular embodiments, the anti-CD3 antibody or antigen-binding fragment may comprise: (A) a VH polypeptide comprising (i) the CDR-H1, the CDR-H2, and the CFR-H3 sequences of SEQ ID NOS: 512, 514, and 516, respectively and (ii) an amino acid sequence at least 90% identical to SEQ ID NO: 510; and (B) a VL polypeptide comprising (i) the CDR-L1, the CDR-L2, and the CFR-L3 sequences of SEQ ID NOS: 522, 524, and 526, respectively; and (ii) an amino acid sequence at least 90% identical to SEQ ID NO: 520.

In particular embodiments, the anti-CD3 antibody or antigen-binding fragment may comprise VH and VL polypeptides comprising the amino acid sequences of SEQ ID NOS: 610 and 620, respectively.

In particular embodiments, the anti-CD3 antibody or antigen-binding fragment may comprise VH and VL polypeptides comprising the amino acid sequences of SEQ ID NOS: 510 and 520, respectively.

In particular embodiments, the anti-CD3 antibody or antigen-binding fragment may comprise VH and VL polypeptides comprising the amino acid sequences of SEQ ID NOS: 410 and 420, respectively.

In particular embodiments, the anti-CD3 antibody or antigen-binding fragment may comprise VH and VL polypeptides comprising the amino acid sequences of SEQ ID NOS: 310 and 320, respectively.

In particular embodiments, the anti-CD3 antibody or antigen-binding fragment may comprise VH and VL polypeptides comprising the amino acid sequences of SEQ ID NOS: 710 and 720, respectively.

In particular embodiments, the anti-CD3 antibody or antigen-binding fragment may comprise VH and VL polypeptides comprising the amino acid sequences of SEQ ID NOS: 810 and 820, respectively.

In particular embodiments, the anti-CD3 antibody or antigen-binding fragment may comprise VH and VL polypeptides comprising the amino acid sequences of SEQ ID NOS: 910 and 920, respectively.

In particular embodiments, the anti-CD3 antibody or antigen-binding fragment may comprise VH and VL polypeptides comprising the amino acid sequences of SEQ ID NOS: 1010 and 1020, respectively.

In particular embodiments, the anti-CD3 antibody or antigen-binding fragment may comprise VH and VL polypeptides comprising the amino acid sequences of SEQ ID NOS: 1110 and 1120, respectively.

In particular embodiments, the anti-CD3 antibody or antigen binding fragment may comprise VH and VL polypeptides comprising the amino acid sequences of SEQ ID NOS: 1210 and 1220, respectively.

In particular embodiments, the anti-CD3 antibody or antigen-binding fragment may comprise VH and VL polypeptides comprising the amino acid sequences of SEQ ID NOS: 1310 and 1320, respectively.

In some embodiments, the anti-CD3 antibody or antigen-binding fragment may comprise an antibody constant region, a CH1 domain, a hinge, a CH2 domain, and/or a CH3 domain. In some cases, the antibody constant region, a CH1 domain, a hinge, a CH2 domain, and/or a CH3 domain may individually be of or derived from an IgG or human IgG. In certain cases, the antibody constant region, a CH1 domain, a hinge, a CH2 domain, and/or a CH3 domain may individually be of or derived from a human IgG1, IgG4, IgG2, or IgG3.

In some embodiments, the ani-CD3 antibody or antigen-binding fragment may comprise a fragment crystallizable (Fc) region.

In certain embodiments, the Fc region may be of a human IgG1 In some cases, the Fc region may comprise one or more of the following amino acid modifications: N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E4300, S440Y M428L, N434S, L328F, M252Y, S254T, T256E, or any combination thereof, according to ETU numbering.

In certain embodiments, the Fc region may be of a human IgG4. In some cases, the Fe region may comprise one or more of the following amino acid modifications: E233P, F234V, L235A, G237A, E318A, S228P, L236E, S241P, L248E, T394D, M252Y, S254T, T256E, N297A, N297Q, or any combination thereof, according to EU numbering.

In certain embodiments, the Fc region may be of a human IgG2 In some cases, the Fc region may comprise one or more of the following amino acid modifications: P238S, V234A, G237A, H268A, H268Q, H268E, V309L, N297A, N297Q, A330S, P331S, C232S, C233S, M252Y, S254T, T256E, or any combination thereof, according to EU numbering.

In certain embodiments, the Fe region may be of a human IgG3. In some cases, the Fe region may comprise E235Y, according to EU numbering.

In some embodiments, the ani-CD3 antibody or antigen-binding fragment may comprise an IgG, IgA, IgE, IgD, or IgM. In some cases, the IgG may be IgG1, IgG4, IgG2, or IgG3.

In some embodiments, the anti-CD3 antibody or antigen-binding fragment may comprise an antibody fragment selected from the group consisting of: a fragment antigen-binding (Fab); an Fab₂; an Fab₃; an Fab′ fragment; an F(ab′)₂; a variable fragment (Fv); a single-chain Fv (scFv) fragment; a diabody; a triabody; a minibody; a scFv-Fc, a scFv2-Fc2; scFv-IgG; a monovalent IgG (or a half IgG); and/or a chimeric antigen receptor (CAR) comprising an antigen-binding region comprising said VH polypeptide and/or said VL polypeptide, a transmembrane domain, and at least one intracellular signaling domain (optionally derived from a T-cell receptor, further optionally CD3ζ).

In some embodiments, the anti-CD3 antibody or antigen-binding fragment may comprise or may be comprised in a multispecific antibody or antibody fragment having at least (a) a first antigen-binding region specific to CD3, comprising said VH polypeptide and/or said VL polypeptide, and (b) a second antigen-binding region.

In certain embodiments, the second antigen-binding region may be specific to an oncology target, a target molecule expressed on cancer cells, an immune-oncology target, a target molecule expressed on immune cells, an autoimmune disorder target (optionally a self-reactive immune molecule or a target molecule expressed on an immune cell expressing a self-reactive immune molecule), an inflammatory disease target (optionally an inflammatory cytokine or chemokine or a receptor thereof), a neurodegenerative disease target, an infectious disease target (optionally a target molecule of a virus, bacterium, or a fungus), a target molecule expressed on infected cells (optionally infected with a virus, a bacterium, or fungus), a metabolic disease target, a cognitive disorder target, a blood-brain barrier target, or a blood disease target.

In certain embodiments, the second antigen-binding region may be specific to a second antigen selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-L ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10CCR, CCR1, CCR10, CCR10, CCR2, CCR3, CCR4, CCR5, CCR6, CCR7, CCR8, CCR9, CD1, CD2; CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1) Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcRI, FEN-1, Ferritin, FGF, FGF-19, FGF-2, FGF3, FGF-8, FGFR, FGFR-3, Fibrin, FL, FLIP, Fit-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3-GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, FILA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, inhibin, iNOS, Insulin A-chain, Insulin B-chain. Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lpin, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPTF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX-40L, OX40R, p150, p95, PADPr, Parathyroid hormone, PARC, PARP, PBR, PBSF, PCAD, P-Cadherin, PCNA, PDGF, PDGE, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shb, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., I-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, INF-RII, TNFRSF10A (TRAIL R1, Apo-2, DR4), TNFRSF10B (TRAIL R2 DR5, KILLER, TRICK-2A, TRICK-B), TNFRSF10C (TRAIL R3, DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR ATTR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF36B (DcR3 M68, TR6), TNFRSF7 (CD27), TNFRSF8 (CD30), TNFRSF9 (4-1BB CD137, ILA), TNFRSF21 (DR6), TNFRSF22 DcTRAIL R2 TNFRH2), TNFRSF23 (Dc TRAIL R1, TNFR1H1), TNFRSF25 (DR3 Apo-3, LARD, TR-3, TRAMP, WSL-1), TNFSF10 (TRAIL, Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSF1A (TNF-a Connectin, DIF, TNFSF2), TNFSF1B (INF-b LTa, TNFSF1), TNFSF3 (LTb TNFC, p33), TNFSF4 (OX40 Ligand gp34, TXGP1), TNFSF5 (CD40 Ligand, CD154, gp39, HIGM1, IMD3, TRAP), TNFSF6 (Fas Ligand Apo-1 Ligand, APT1 Ligand), TNFSF7 (CD27 Ligand, CD70), TNFSF8 (CD30 Ligand CD1A53), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (ft-4), VEGI, VFM, Viral antigens, VLA, VLA-1, VLA-4, VNR integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1) LAG-3 (lymphocyte activation gene-3), TIM-3 (T cell immunoglobulin and mucin protein-3), and hormone receptors and growth factors.

In certain embodiments, the second antigen-binding region may be specific to an antigen selected from the group consisting of BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcγRIIIa (CD16), FcγRIIa (CD32a), FcγRIIb (CD32b), FcγRI (CD64) Toll-like receptors (TLRs), TLR4, TLR9, cytokines, IL-2, IL-5, IL-13, IL-6, IL-17, IL-12, IL-23, TNFa, TGFb, cytokine receptors, IL-2R, chemokines, chemokine receptors, growth factors, VEGF, and HGF.

In certain embodiments, the multispecific antibody or antibody fragment may be bispecific. In certain embodiments, the multispecific antibody or antibody fragment may further comprise a third antigen-binding region. In certain embodiments, the multispecific antibody or antibody fragment may be trispecific.

In certain embodiments, the multispecific antibody or antibody fragment may comprise a multispecific format selected from the group consisting of: Fab-Fc-scFv, scFv2-Fc2, scFv-IgG, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

In certain embodiments, the multispecific antibody or antibody fragment may comprise at least one CLκ-preferring variant CH1 domain, optionally a CLκ-preferring variant CH1 domain described in WO2021067404.

In certain embodiments, the multispecific antibody or antibody fragment may comprise at least one CLκ-preferring variant CH1 domain, optionally a CLλ-preferring variant CH1 domain described in WO2021067404.

In certain embodiments, the multispecific antibody or antibody fragment may comprise at least one pair of a variant CH1 domain and a variant CL domain which preferentially pair with each other, optionally a pair described in WO2022150787.

In certain embodiments, the multispecific antibody or antibody fragment may comprise at least one pair of a variant CH3 domain and another variant CH3 domain which preferentially pair with each other, optionally a pair described in WO2022150785.

In some embodiments, the anti-CD3 antibody or antigen-binding fragment may bind to CD3 with a greater binding affinity or avidity at an acidic pH, optionally about pH 6.0, than at a physiological pH, optionally about pH 7.4. In certain embodiments, the binding affinity or avidity is measured via: (1) surface plasmon resonance (SPR), optionally using a BIACORE® system (GE healthcare or CYTIVA®); (2) bio-layer interferometry (BLI), optionally using an OCTET® system; (3) enzyme-linked immunosorbent assay (ELISA); and/or (4) radioimmunoassay (RIA). In certain embodiments, the CD3 may be: (1) a human CD3, optionally CD3εδ; and/or (2) a non-human primate, optionally monkey, further optionally cynomolgus CD3, optionally CD3εδ. In certain embodiments, the binding to CD3 may be at least ×1.2, at least ×1.5, at least ×2, at least ×5, at least ×10, at least ×20, at least ×50, at least ×100, at least ×10³, at least ×10⁴, at least ×10⁵, at least ×10⁶, at least ×10⁷, at least ×10⁸, or at least ×10⁹ greater at an acidic pH, optionally about pH 6.0, than at a physiological pH, optionally about pH 7.4, based on equilibrium dissociation constant (Kd) values. In certain embodiments, the anti-CD3 antibody or antigen-binding fragment may not bind to CD3 at a physiological pH, optionally about pH 7.4.

In some embodiments, the anti-CD3 antibody or antigen-binding fragment may bind to human CD3 (optionally CD3εδ) at an acidic pH (optionally about pH 6.0).

In certain embodiments, the anti-CD3 antibody or antigen-binding fragment may bind to human CD3 (optionally CD3εδ) at an acidic pH (optionally about pH 6.0) with a Kd value lower than 1.57×10⁸ (M), lower than 1.0×10⁸ (M), lower than 9.0×10⁹ (M), lower than 8.0×10⁹ (M), lower than 7.0×10⁹ (M), lower than 6.0×10⁹ (M), lower than 5.0×10⁹ (M), lower than 4.0×10⁹ (M), lower than 3.0×10⁹ (M), lower than 2.0×10⁹ (M), lower than 1.0×10⁹ (M), lower than 9.0×10¹⁰ (M), or lower than 8.0×10¹⁰ (M). In some cases, the Kd value may be measured via SPR, optionally using a BIACORE® system.

In certain embodiments, the anti-CD3 antibody or antigen-binding fragment may bind to human CD3 (optionally CD3εδ) at an acidic pH (optionally about pH 6.0) with a Kd value between 1.56×10⁸ (M) and 7.0×10¹⁰ (M), between 1.0×10⁸ (M) and 7.0×10¹⁰ (M), between 1.0×10⁸ (M) and 1.0×10⁹ (M), between 2.0×10⁸ (M) and 1.0×10⁹ (M), or between 5.0×10⁸ (M) and 1.0×10⁹ (M). In some cases, the Kd value may be measured via SPR, optionally using a BIACORE® system.

In certain embodiments, the anti-CD3 antibody or antigen-binding fragment may bind to human CD3 (optionally CD3εδ) at an acidic pH (optionally about pH 6.0) with a Kd value lower than 9.33×10⁹ (M), lower than 9.0×10⁹ (M), lower than 8.0×10⁹ (M), lower than 7.0×10⁹ (M), lower than 6.0×10⁹ (M), lower than 5.0×10⁹ (M), lower than 4.0×10⁹ (M), lower than 3.0×10⁹ (M), or lower than 2.0×10⁹ (M). In some cases, the Kd value may be measured via BLI, optionally using an OCTET® system.

In certain embodiments, the anti-CD3 antibody or antigen-binding fragment may bind to human CD3 (optionally CD3εδ) at an acidic pH (optionally about pH 6.0) with a Kd value between 9.32×10⁹ (M) and 1.0×10⁹ (M), between 9.0×10⁹ (M) and 1.0×10⁹ (M), between 8.0×10⁹ (M) and 3.0×10⁹ (M), between 7.0×10⁹ (M) and 4.0×10⁹, or between 6.0×10⁹ (M) and 5.0×10⁹ (M). In some cases, the Kd value may be measured via BLI, optionally using an OCTET® system.

In some embodiments, the anti-CD3 antibody or antigen-binding fragment may bind to cynomolgus CD3 (optionally CD3εδ) at an acidic pH (optionally about pH 6.0).

In certain embodiments, the anti-CD3 antibody or antigen-binding fragment may bind to cynomolgus CD3 (optionally CD3εδ) at an acidic pH (optionally about pH 6.0) with a Kd value lower than 2.0×10⁸ (M), lower than 1.0×10⁸ (M), lower than 9.0×10⁹ (M), lower than 8.0×10⁹ (M), lower than 7.0×10⁹ (M), lower than 6.0×10⁹ (M), lower than 5.0×10⁹ (M), lower than 4.0×10⁹ (M), lower than 3.0×10⁹ (M), or lower than 2.0×10⁹ (M). In some cases, the Kd value may be measured via BLI, further optionally using an OCTET® system.

In certain embodiments, the anti-CD3 antibody or antigen-binding fragment may bind to cynomolgus CD3 (optionally CD3εδ) at an acidic pH (optionally about pH 6.0) with a Kd value between 2.0×10⁸ (M) and 1.0×10⁹ (M), between 1.0×10⁸ (M) and 1.0×10⁹ (M), between 9.0×10⁹ (M) and 2.0×10⁹ (M), or between 8.0×10⁹ (M) and 5.0×10⁹. In some cases, the Kd value may be measured via BLI, further optionally using an OCTET® system.

In some embodiments, the anti-CD3 antibody or antigen-binding fragment may bind to CD3-expressing cells with greater binding at an acidic pH (optionally about pH 6.0) than at a physiological pH (optionally about pH 7.4).

In certain embodiments, the cell binding may be measured via flow cytometry, further optionally based on median fluorescence intensity (MFI). In certain embodiments, the cells may express a human CD3, optionally CD3εδ. In certain embodiments, the cells may express a non-human primate (optionally monkey, further optionally cynomolgus) CD3 (optionally CD3εδ). In certain embodiments, the cells may be primary cells. In certain embodiments, the cells may be cells of a cell line. In certain embodiments, the cells may be human cells. In certain embodiments, the cells may be human T cells. In certain embodiments, the cells may be Jurkat cells. In certain embodiments, the cells may be non-human primate (optionally monkey, further optionally cynomolgus) cells. In certain embodiments, the cells may be HSC-F cells.

In certain embodiments, the binding to CD3-expressing cells may be at least ×1.2, at least ×1.5, at least ×2, at least ×5, at least ×10, at least ×20, at least ×50, at least ×100, at least ×10³, at least ×10⁴, at least ×10⁵, at least ×10⁶, at least ×10⁷, at least ×10⁸, or at least ×10⁹ greater at an acidic pH (optionally about pH 6.0) than at a physiological pH (optionally about pH 7.4) based on NCB values calculated using MFI values when measured via flow cytometry. In some cases, NCB value is calculated as {(Sample MFI)−(Secondary Only MFI)}/(Secondary Only MFI), in other words, {(MFI for “incubation with test antibody followed by secondary antibody”)−(MFI for incubation with no primary antibody followed by secondary antibody)}/(MFI for incubation with no primary antibody followed by secondary antibody).

In certain embodiments, the binding to CD3-expressing cells may not bind to CD3-expressing cell, at a physiological pH (optionally about pH 7.4).

In some embodiments, the anti-CD3 antibody or antigen-binding fragment may, upon binding to CD3 on cells (optionally T cells), elicits activation and/or enhances cell cytotoxic function(s) of the cells.

In certain embodiments, the elicitation of T cell activation or T cell killing may occur while displaying a decreased propensity to elicit cytokine production to levels capable of inducing cytokine release syndrome (CRS). In certain embodiments, upon binding to CD3 on cells, (optionally T cells), the anti-CD3 antibody or antigen-binding fragment may not elicit cytokine production upon binding to CD3 to levels capable of inducing CRS.

In some embodiments, the anti-CD3 antibody or antigen-binding fragment may comprise or may be comprised in a multispecific antibody or antibody fragment having at least (a) a first antigen-binding region specific to CD3, comprising said VH polypeptide and/or said VL polypeptide, and (b) a second antigen-binding region specific to a second antigen; and upon binding to (i) CD3 on a first cell (optionally T cells) and (ii) the second antigen expressed on a second cell, the first cell may exhibit cytotoxicity to the second cell.

In another aspect, the disclosure provides nucleic acids (e.g., isolated or recombinant nucleic acids, one or more nucleic acids, such as a nucleic acid, a combination of two or more nucleic acids, and the like) encoding any of the anti-CD3 antibodies and antigen-binding fragments described herein. Such nucleic acids may include DNA such as cDNA or RNA such as mRNA, e.g., for delivery to cells and expression of anti-CD3 antibodies or antigen-binding fragments.

In some embodiments, a nucleic acid according to the present disclosure may comprise: (A) a VH polypeptide-encoding nucleic acid sequence at least 80%, at least 85%, at least 90%, at least 92%, at least 94%, at least 95%, at least 96%, at least 97%, at least 98%, at least 90%, or 100% identical to SEQ ID NO: 650, 550, 450, 350, 750, 850, 950, 1050, 1150, 1250, or 1350, or a RNA (e.g., mRNA) version of any of the foregoing; and/or (B) a VL polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 660, 560, 460, 360, 760, 860, 960, 1060, 1160, 1260, or 1360, or a RNA (e.g., mRNA) version of any of the foregoing.

In certain embodiments, said nucleic acid may comprise VH polypeptide-encoding and VL polypeptide-encoding nucleic acid sequences of SEQ ID NOS: 650 and 660, respectively.

In certain embodiments, said nucleic acid may comprise VH polypeptide-encoding and VL polypeptide-encoding nucleic acid sequences of SEQ ID NOS: 550 and 560, respectively.

In certain embodiments, said nucleic acid may comprise VH polypeptide-encoding and VL polypeptide-encoding nucleic acid sequences of SEQ ID NOS: 450 and 460, respectively.

In certain embodiments, said recombinant nucleic acid may comprise VH polypeptide-encoding and VL polypeptide-encoding nucleic acid sequences of SEQ ID NOS: 350 and 360, respectively.

In certain embodiments, said nucleic acid may comprise VH polypeptide-encoding and VL polypeptide-encoding nucleic acid sequences of SEQ ID NOS: 750 and 760, respectively.

In certain embodiments, said nucleic acid may comprise VH polypeptide-encoding and VL polypeptide-encoding nucleic acid sequences of SEQ ID NOS: 850 and 860, respectively.

In certain embodiments, said nucleic acid may comprise VH polypeptide-encoding and VL polypeptide-encoding nucleic acid sequences of SEQ ID NOS: 950 and 960, respectively.

In certain embodiments, said nucleic acid may comprise VH polypeptide-encoding and VL polypeptide-encoding nucleic acid sequences of SEQ ID NOS: 1050 and 1060, respectively.

In certain embodiments, said nucleic acid may comprise VL polypeptide-encoding and VL polypeptide-encoding nucleic acid sequences of SEQ ID NOS: 1150 and 1160, respectively.

In certain embodiments, said nucleic acid may comprise VH polypeptide-encoding and VL polypeptide-encoding nucleic acid sequences of SEQ ID NOS: 1250 and 1260, respectively.

In certain embodiments, said nucleic acid may comprise VH polypeptide-encoding and VL polypeptide-encoding nucleic acid sequences of SEQ ID NOS: 1350 and 1360, respectively.

In some cases, the nucleic acid may comprise an RNA (e.g., mRNA) which may comprise VH polypeptide-encoding and VL polypeptide-encoding RNA sequences corresponding to any of the foregoing nucleic acid sequences.

In another aspect, the disclosure provides vectors (e.g., one or more vectors, such as a vector, a combination of two or more vectors, and the like) encoding any of the anti-CD3 antibodies and antigen-binding fragments described herein.

In some embodiments, the vector may comprise any of the nucleic acids described herein.

In some embodiments, the vector may be an expression vector.

In some embodiments, the vector may comprise a plasmid, a viral vector (optionally adenoviral, lentiviral, or retroviral), a lipid-based vector, a self-replicating RNA vector, a virus-like particle, a polymer-based vector, and/or a nanoparticle, optionally a lipid-based nanoparticle.

In another aspect, the disclosure provides isolated or recombinant cells comprising, transfected with, transformed with, or transduced with any of the nucleic acids and/or any of the vectors described herein.

In some embodiments, the isolated or recombinant cell may be mammalian. In certain embodiments, the isolated or recombinant cell may be human, non-human primate, monkey, rabbit, rodent, hamster, rat, or mouse. In some embodiments, the isolated or recombinant cell may be non-mammalian, optionally plant, bacterial, fungal, yeast, protozoa, or insect. In some embodiments, the isolated or recombinant cell may be an immune cell or a hybridoma.

In another aspect, the disclosure provides pharmaceutical compositions.

In some embodiments, a pharmaceutical composition according to the present disclosure may comprise: (A) any of the anti-CD3 antibodies and antigen-binding fragments described herein; and (B) a pharmaceutically acceptable carrier and/or excipient. In some embodiments, a pharmaceutical composition according to the present disclosure may comprise: (A) any of the nucleic acids described herein; and (B) a pharmaceutically acceptable carrier and/or excipient. In some embodiments, a pharmaceutical composition according to the present disclosure may comprise: (A) any of the vectors described herein; and (B) a pharmaceutically acceptable carrier and/or excipient. In some embodiments, a pharmaceutical composition according to the present disclosure may comprise: (A) any of the isolated or recombinant cells described herein, and (B) a pharmaceutically acceptable carrier and/or excipient.

In another aspect, the disclosure provides methods of treating a subject in need of such treatment and methods of treating or preventing a disease, disorder, or a condition in a subject.

In some embodiments, the method may comprise administering to the subject art effective amount of any of the anti-CD3 antibodies and antigen-binding fragments described herein. In some embodiments, the method may comprise administering to the subject an effective amount of any of the nucleic acids described herein. In some embodiments, the method may comprise administering to the subject an effective amount of any of the vectors described herein. In some embodiments, the method may comprise administering to the subject an effective amount of any of the isolated or recombinant cells described herein. In some embodiments, the method may comprise administering to the subject an effective amount of any of the pharmaceutical compositions described herein.

In another aspect, the disclosure provides methods of eliciting cytotoxicity to a cell expressing a target molecule of interest.

In some embodiments, the method may comprise administering to the subject an effective amount of any of the anti-CD3 antibodies and antigen-binding fragments comprising or comprised in a multispecific antibody or antibody fragment described herein. In some embodiments, the method may comprise administering to the subject an effective amount of a nucleic acid encoding such an anti-CD3 antibody or antigen-binding fragment. In some embodiments, the method may comprise administering to the subject an effective amount of a vector comprising such a nucleic acid and/or encoding such an anti-CD3 antibody or antigen-binding fragment. In some embodiments, the method may comprise administering to the subject an effective amount of isolated or recombinant cells comprising, transfected with, transformed with, or transduced with such a nucleic acid or such a vector. In some embodiments, the method may comprise administering to the subject an effective amount of a pharmaceutical composition comprising (A) any of the anti-CD3 antibodies and antigen-binding fragments comprising or comprised in a multispecific antibody or antibody fragment described herein, a nucleic acid encoding such an anti-CD3 antibody or antigen-binding fragment, a vector comprising such a nucleic acid, and/or isolated or recombinant cell comprising, transfected with, transformed with, or transduced with such a nucleic acid or such a vector, and/or (B) a pharmaceutically acceptable carrier and/or excipient.

In any of the above embodiments of methods, in some cases, the subject may be a mammal, optionally a human, a non-human primate, a monkey, a horse, a cow, sheep, a goat, a pig, a dog, a cat, a rabbit, a rodent, a hamster, a rat, or a mouse. In certain embodiments, the subject may be a non-mammalian vertebrate, optionally a bird, fish, an amphibian, or a reptile.

In any of the above embodiments of methods, in some cases, the method may further comprise administering to the subject an additional agent, optionally an adjuvant or a therapeutic agent.

In any of the above embodiments of methods, in some cases, the subject may comprise or have a risk of developing a disease, disorder, or a condition.

In any of the above embodiments of methods, in some cases, the disease, disorder, or a condition comprises cancer or a neoplastic condition, an autoimmune disease, a neurodegenerative disease, an infectious disease, an inflammatory disease, or another disease.

In certain embodiments, the cancer may be a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

In certain embodiments, the cancer may be a liquid cancer, optionally chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmacytoma of bone, extraosseous plasmacytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK-large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In certain embodiments, the autoimmune or inflammatory disease may be psoriasis, rheumatoid arthritis, autoimmune arthritis, type I diabetes, sarcoidosis, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren syndrome, Addison's disease, Behcet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis.

In certain embodiments, the neurodegenarative disease may be Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease.

In certain embodiments, the infectious disease may be a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter pylori, Neisseria gonorrhoea, Vibrio vulnificus, and/or (3) the fungal disease is Aspergillosis, Candida, Candida auris, Cryptococcus neoformans, Pneumocystis jirovecii, Mucormycoses, Talaromyces, ringworm, Blastomyces, Coccidioides, Cryptococcus gattii, Histoplasma, Paracoccidioides, or Sporothrix infection.

In another aspect, the disclosure provides methods of manufacturing an anti-CD3 antibody or antigen-binding fragment described herein.

In some embodiments, the method may comprise (a) culturing cells comprising a nucleic acid encoding the ani-CD3 antibody or antigen-binding fragment in a condition that allows for expression of said antibody or antigen-binding fragment, and (b) harvesting and purifying the antibody or antigen-binding fragment from the cell culture from (a).

In another aspect, the disclosure provides methods of manufacturing an isolated or recombinant cell according to the present disclosure or a population of such cells.

In some embodiments, the method may comprise introducing (i) a nucleic acid encoding an anti-CD3 antibody or antigen-binding fragment according to the present disclosure and/or (ii) a vector encoding an anti-CD3 antibody or antigen-binding fragment according to the present disclosure or comprising such a nucleic acid into one or more cells.

In certain embodiments, the inducing may occur in vitro, ex vivo, or in vivo.

Any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure may be for use in medicine or in the preparation of a medicament for use in medicine.

Any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure may be for use in treating a disease, disorder, or condition, optionally any of the diseases, disorders, or conditions described herein.

The present disclosure further encompass use of any of the anti-CD3 antibodies and antigen-binding fragments according to the present disclosure, any of the nucleic acids according to the present disclosure, any of the vectors according to the present disclosure, any of the isolated or recombinant cells according to the present disclosure or a population of such cells, and/or any of the pharmaceutical compositions according to the present disclosure for the manufacture of a medicament for treatment of a disease, disorder, or condition, optionally any of the diseases, disorders, or conditions described herein.

### DETAILED DESCRIPTION

### Definitions

Unless defined otherwise, all technical and scientific terms used herein have the same meaning as commonly understood by one of ordinary skill in the art to which this disclosure belongs.

It is to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

Unless the context clearly dictates otherwise, the singular forms “a,” “an,” and “the” as used herein and in the appended claims include plural refence. Thus, the reference to “a cell” refers to one or more cells and equivalents thereof known to those skilled in the art, and so forth.

As used herein, the term “about,” when used in reference to a particular recited numerical value, means that the value may vary from the recited value by no more than 5%. For example, as used herein, the expression “about 100” includes 95 and 105 and all values in between (e.g., 96, 97, 98, 99, 99.1, 99.2, 99.3, 99.4, 100, 101, 102, 103, 104, etc.).

It is understood that aspects and embodiments of the disclosure described herein include “comprising,” “consisting,” and “consisting essentially of” aspects and embodiments. Transitional phrases “comprising,” “including,” “having,” “containing,” “involving,” “composed of,” and the like, are to be understood to be open-ended, i.e., do not exclude additional, unrecited elements. Therefore, such transitional phrases encompass both open-ended embodiments and, as appropriate, closed and semi-closed embodiments (i.e., embodiments described with “consisting of” and “consisting essentially of”, respectively). Only the transitional phrases “consisting of” and “consisting essentially of” shall be closed or semi-closed transitional phrases, respectively.

The term “antibody” is used herein in the broadest sense and encompasses various antibody structures, including but not limited to intact antibodies and antibody fragments (preferably those fragments that exhibit the desired antigen-binding activity (i.e., antigen-binding fragment)), multispecific (e.g., bispecific, trispecific, etc.) antibodies and antibody fragments, monoclonal antibodies, polyclonal antibodies, and the like.

The terms “intact antibody” and “whole antibody” or the like are used herein interchangeably and refer to an antibody having a structure substantially similar to a native antibody. In some instances, an antibody comprises heavy (H) and light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. For example, an intact IgG (or IgD or IgE) antibody comprises two immunoglobulin heavy chains and two immunoglobulin light chains. Therefore, in some instances, an antibody according to the present disclosure may comprise two pairs of heavy and light chains interconnected by disulfide bonds, or an antigen-binding fragment(s) thereof. Some intact antibody comprises multiple units each comprising two pairs of heavy and light chains interconnected by disulfide bonds. For example, art intact IgA comprises two units and art intact IgM comprises five units. Therefore, in other instances, an antibody according to the present disclosure may instead comprise multiple (e.g., two, three, four, five, and so on) units each comprising two pairs of heavy and light chains interconnected by disulfide bonds, or an antigen-binding fragment(s) thereof.

Each heavy chain is comprised of: a heavy chain variable domain (VH); and a heavy chain constant region (CH), which is typically comprised of domains CH1, CH2 and CH3. Each light chain is comprised of: a light chain variable domain (VL); and a light chain constant domain (CL). Typically (with some exceptions such as nanobodies, camelid heavy chain antibodies, IgNARs, and the like), one VH and one VL may form an antigen-binding region. The VH and VL can be further subdivided into regions of hypervariability, termed complementarity determining regions (CDRs), interspersed with regions that are more conserved, termed framework regions (FRs). Each VH and VL polypeptide is composed of three CDRs and four FRs, arranged from amino-terminus to carboxy-terminus in the following order: FR1, CDR1, FR2, CDR2, FR3, CDR3, FR4, CDRs in a heavy chain are designated “CDR-H1,” “CDR-H2,” and “CDR-H3,” respectively, and the CDRs in a light chain are designated “CDR-L1,” “CDR-L2,” and “CDR-L3.” FRs in a heavy chain are designated “FR-H1,” “FR-H2,” “FR-3,” and “FR-H4,” respectively, and the FRs in a light chain are designated “FR-L1” “FR-L2,” “FR-L3,” and “FR-L4.” In certain embodiments of the disclosure, the FRs of the antibody (or antigen-binding fragment thereof) may be identical to the human germline-encoded sequences (e.g., heavy chain FR sequences encoded by the VH1-03 germline, and/or light chain FR sequences encoded by the VK4-01 germline) or may be naturally or artificially modified. An amino acid consensus sequence may be defined based on a side-by-side analysis of two or more CDRs.

The numbering of amino acid residues in antibody variable and/or constant domains may be performed by any appropriate numbering schemes, methods, and definitions, e.g., based on numbering schemes such as EU numbering (as described in Kabat et al., Sequences of Proteins of Immunological interest, 5th Ed. Public Health Service, National Institutes of Health, Bethesda, Md. (1991)), IMGT numbering, Kabat numbering, Chothia numbering, Martin numbering, Gelfand numbering, or Honneger's numbering); or structurally (see e.g., NCBI online tool, IgBlast; Dondelinger et al., Front Immunol. 2018 Oct. 16; 9:2278).

According to TMGT (the international ImMunoGeneTics information system for immunoglobulins or antibodies, T cell receptors, MH, immunoglobulin superfamily IgSF and MhSF), the CH1 domain, the hinge region, the CH2 domain, and the CH3 domain correspond to the amino acid positions 118-215, 216-230, 231-340, and 341-446, respectively (EU numbering). The terms “CH1 domain”, “hinge”, “CH2 domain”, and “CH3” are used in a broad sense herein to encompass any naturally occurring, corresponding heavy chain constant domain and/or region allotypes and variants thereof, which may comprise fewer or more amino acids e.g., a CH1 domain may comprise a portion of a hinge region) and/or amino acid modification(s).

An exemplary CH1 domain of a human IgG1 may comprise the amino acid sequence of SEQ ID NO: 41 or 42; an exemplary hinge of a human IgG1 may comprise the amino acid sequence of SEQ ID NO: 51; and a CH2 domain of a human IgG1 may comprise the amino acid sequences of SEQ ID NOS: 61. An exemplary CH3 domain of a human IgG1 may comprise the amino acid sequence of SEQ ID NO: 71, 72, 73, or 74, and a C-terminal K may be added to any of such CH3 sequences. Any variants of such exemplary sequences may be used in conjunction with anti-CD3 variable sequences described herein.

“Fc region” is a C-terminal region of an immunoglobulin heavy chain that contains at least a portion of the constant region, including native sequence Fc regions and variant Fc regions. A human IgG heavy chain Fc region can extend from Cys226, or from Pro230, to the carboxyl-terminus of the heavy chain. However, the C-terminal lysine (Lys447) of the Fe region may or may not be present. Unless otherwise specified herein, numbering of amino acid residues in the Fe region or constant region is according to the EU numbering system, also called the EU index, as described in Kabat et al., Sequences of Proteins of Immunological Interest, 5th Ed, Public Health Service, National Institutes of Health, Bethesda, Md., 1991.

The phrase “Effector function” of an antibody refers to biological activities attributable to the F, region of an antibody, which varies by antibody isotype. Exemplary effector functions include: complement (e.g., C1q) binding and complement dependent cytotoxicity (CDC); Fe receptor binding; antibody-dependent ceil-mediated cytotoxicity (ADCC); phagocytosis; down regulation of cell surface receptors (e.g., B cell receptor); and B cell activation.

There are two major light chain isotypes, kappa (κ) and lambda (λ), and the corresponding light chain constant domains are called kappa CL, domain (CLκ domain) and lambda CL domain (CLλ domain), respectively.

According to IMGT, the CLκ domain is the amino acid positions 108-214 (EU numbering). An exemplary CLκ domain of a human IgG may comprise the amino acid sequence of SEQ ID NO: 81. According to IMGT the CLλ domain is the amino acid positions 107-215 (EU numbering). An exemplary CL λ domain of a human IgG may comprise the amino acid sequence of SEQ ID NO: 82.

The terms “CLκ domain” “CLλ domain” are used in a broad sense herein to encompass any naturally occurring, corresponding light chain constant domain and/or region allotypes and variants thereof, which may comprise fewer or more amino acids and/or amino acid modification(s).

Various standard sequences (corresponding to different allotypes) of the constant domains of human IgG1, IgG2, IgG3, and IgG4 are known in the field and may be found for example in Vidarsson et al., Front Immunol. 2014 Oct. 20:5-520 and U.S. Pat. No. 9,150,663, the disclosures of which are hereby incorporated by reference herein in their entirety herein. Again these reference sequences are intended to be exemplary as Applicant intends for human IgG1, IgG2, IgG3, and IgG4 sequences to include any naturally occurring human IgG1, IgG2, IgG3, and IgG4 allotype.

An “antigen-binding fragment” or “antigen-binding antibody fragment” refers to a portion of an intact antibody or to a combination of portions derived from one or more intact antibody that binds the antigen to which the intact antibody binds (in this case, CD3). An antigen-binding fragment of an antibody includes any naturally occurring, enzymatically obtainable, synthetic, or genetically engineered polypeptide or glycoprotein that specifically binds an antigen to form a complex. Exemplary antigen-binding fragments include, but are not limited to, Fv, Fab, Fab′, Fab′-SH, F(ab′)₂, diabodies; linear antibodies; single-chain antibody molecules (e.g. single-chain variable fragment (scFv), half antibody, nanobody or VH only, or VL only); and multispecific antibodies formed from antibody fragments. In some embodiments, the antigen-binding fragments of the anti-CD3 antibodies described herein are scFvs. The term “half molecule” or “half antibody” when referring to IgG, IgE, or IgD, which may also be referred to as “half IgG”, “half IgE” or “half IgD”, respectively, refers to a set of one heavy chain and one light chain of the referenced antibody.

An “antigen-binding region” refers to a portion of an antibody or antigen-binding fragment with specificity for an antigen.

With regard to multispecific antibodies (e.g., bispecific, trispecific, tetraspecific, and so on), such antibodies comprise at least two different antigen binding regions which recognize and specifically bind to at least two different antigens or epitopes. The at least two epitopes may or may not be within the same antigen. A “bispecific antibody” is a type of multispecific antibody and comprises two different antigen binding regions which recognize and specifically bind to two different antigens or two epitopes. A bispecific antibody may target, for example, two different surface receptors on the same or different (e.g., an immune cell and a cancer cell) cells.

The phrase “different antigens” may refer to different and/or distinct proteins, polypeptides, or molecules; as well as different and/or distinct epitopes, which epitopes may be contained within one protein, one polypeptide, or one molecule.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

A “monoclonal antibody” or “mAb” refers to an antibody obtained from a population of substantially homogeneous antibodies, i.e., the individual antibodies comprising the population are identical and/or hind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single.

“Cluster of Differentiation 3” or “CD3”, generally refers to any native CD3 from any vertebrate source, including mammals such as primates (e.g., humans and non-human primates) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No NP_000724), which is 207 amino acids in length, and human CD3γ protein (NCBI RefSeq No. NP_000064), which is 182 amino acids in length. The term also refers to either the human or cynomolgus CD3epsilon protein, SEQ ID NOs: 91 and 92, respectively, (Table U). “CD3εN27” and “CD3εN13” refer to the N-terminal 27 amino acids and the N-terminal 13 amino acids, respectively, of CD3, and optionally containing chemical modifications or conjugations made thereto.

An “anti-CD3 antibody” refers to an antibody or an antigen-binding fragment thereof capable of binding to CD3, e.g., CD3ε and/or CD3γ, e.g., human CD3ε; and/or CD3γ with sufficient affinity and/or specificity such that the antibody is useful as a diagnostic and/or therapeutic agent in targeting CD3. In some embodiments, an anti-CD3 antibody binds to CD3 with a dissociation constant (K_{D}) of about 100×10⁻⁹ M or less, about 50×10⁻⁹ M or less, about 25×10⁻⁹ M or less, about 20×10⁻⁹ M or less, or about 10×10⁻⁹ M or less. In some embodiments, an anti-CD3 antibody binds to CD3 with a dissociation constant (K_{D}) of about 5×10⁻⁹ M or less. In some embodiments, an anti-CD3 antibody binds to CD3 with a dissociation constant (K_{D}) of about 2.5×10⁻⁹ M or less. In some embodiments, an anti-CD3 antibody binds to CD3 with a dissociation constant (K_{D}) of about 1×10⁻¹⁰ M or less. In some embodiments, K_{D} is measured by SPR, e.g., BIACORE®, BLI measurements using, e.g., a FORTEBIO OCTET® HTX instrument (Pall Life Sciences), or solution-affinity ELISA. In some embodiments, the KD is measured using an scFv fragment of the anti-CD3 antibody. In some embodiments, the monovalent KD is measured. In some embodiments, the anti-CD3 antibody binds to an epitope of CD3 that is conserved among CD3 from different species, e.g., human and cyno cross-reactive.

An “immunoconjugate” is an antibody conjugated to one or more moiety such as a polymer, a label, an additional agent, e.g., an antibiotic, a second anti-CD3 antibody, a vaccine, or a toxoid, or any other therapeutic moiety, or any of the additional agents described herein.

The term “developable” or “developability” refers to the extent to which one or more polypeptides in a plurality of polypeptides possess desirable characteristics, such as but not limited to: desirable binding specificity, for example binding to a cognate antigen at desirable affinity and not significantly to non-cognate antigens; desirable expression, for example, in mammalian cells; solubility; viscosity; aggregation; chemical and/or physical stability; desirable shelf-life; melting temperature; toxicity; pharmacokinetic profiles, circulation half-life; and clearance characteristics. Such characteristics may serve as indicia, independently, as combinations of sub-sets of such indicia, or in totality, for the likelihood that such one or more polypeptides may be successfully developed as a therapeutic candidate, and ultimately an approved drug. Generally, polypeptides with desirable developability characteristics possess one or more of relatively high solubility, relatively low viscosity, relatively low propensity for aggregation, relatively high chemical stability, relatively high physical stability, relatively long shelf life, relatively high melting temperature, relatively long circulation half-life, relatively low rate of clearance, and the like. By contrast polypeptides with undesirable developability characteristics generally possess one or more of: relatively low solubility, relatively high viscosity, relatively high propensity for aggregation, relatively poor chemical stability, relatively poor physical stability, relatively short shelf life, relatively low melting temperature, relatively short circulation half-life, relatively fast rate of clearance, and the like.

Methods and assays that may be employed to ascertain the degree to which polypeptides, such as anti-CD3 antibodies and/or antigen-binding fragments thereof a described herein, possess desirable developability characteristics are available in the art, and may include but may not be limited to; cytokine release assays; polyspecificity reagent (PSR) assays (WO 2014/179363 and Xu et al., Protein Eng Des Sel, Vol 26, pages 663-670 (2013)); cross interaction chromatography (CIC); self-interaction chromatography (SIC); hydrophobic interaction chromatography (HIC); size exclusion chromatography (SEC); dynamic light scattering (OLS) spectroscopy; photon correlation spectroscopy; quasi-elastic light scattering, circular dichroism (CD), viscosity measurements; whole cell binding; tissue micro array methodologies; ELISA assays such as BVP ELISA assays; AC-SINS assays (Liu et al; MAbs, Vol. 6, pages 483-492 (2014); melting temperature (Tm) assays; differential scanning calorimetry or differential scanning fluorometry (DSF); and the like (see, e.g., He et al., J. Pharm. Sci., Vol. 100(4), pp. 1330-1340 (2011); Wagner et al., Pharm Develop. & Technol (posted online 2012; hyper-text transfer protocol: informahealthcare.com/doi/abs/10.3109/10837450.2011.649851); Hotzel et al., MAbs, Vol. 4(6), pages 753-7601 (2012), Weiqiang et al., J. Pharm. Sci., Vol. 101(5), pp. 1701-1720 (2012); Banks et al., J. Pharm. Sci., Vol. 101(8), pp. 2720-2732 (2012); Lie et al., J. Pharm. Sci., Vol. 94(9), pp. 1928-1948 (2005); and Payne et al., Biopolymers, Vol 85(5), pp. 527-533 (2006)).

A “developability profile” refers to an index that may be assigned to antibodies upon assessing their developability. A developability profile is a measure or metric by which developability of anti-CD3 antibodies may be assessed, compared, and/or ranked. Such developability profiles serve as a measure of the degree of interaction of CD3 binders and antibodies comprising them. The degree of interaction may be assessed by any number of means available in the art that provides an output value that correlates with a strength or affinity of a polypeptide for a moiety to which it is bound. Exemplary means include flow cytometry means, such as fluorescence-activated cell sorting (FACS); enzyme-linked immunosorbent assay (ELISA); quantitative immunoaffinity assays or immunoprecipitation assays; mammalian two-hybrid or yeast two-hybrid assays, and the like. In the context of FACS, as demonstrated in the Examples, a degree of interaction between polypeptides in the plurality and the PSR may be ascertained by generating a mean fluorescence intensity for each polypeptide-PSR interaction that is detected, and then ordering the mean fluorescence intensity in either ascending or descending order, thereby ranking the polypeptides in the plurality according to the relative degree of interaction between each detected polypeptide and the PSR. Such a ranking provides for a ranking of polypeptides of the plurality such that those polypeptides possessing enhanced developability are readily ascertained, as are those polypeptides possessing decreased developability.

A developability profile may also take the form of a normalized score, for example, by normalizing developability of anti-CD3 antibodies described herein to the developability of a standard (or control) antibody, e.g., anti-HEL antibody.

The term “cytokine release syndrome” (or “CRS”) refers to a pro-inflammatory, positive feedback loop between cytokines and immune cells leading to excessive or uncontrolled release of pro-inflammatory cytokines by cells within the immune system (see, e.g., Lee et al., Blood, Vol. 124, pages 188-195 (2014) and Tisoneik et al., Microbiol Mol Biol Rev, Vol. 76, pages 16-32 (2012). Upon stimulation and activation, T cells release a series of cytokines to a level and degree that generates untoward biological/physiological effects or varying degree and severity, including acute inflammation characterized by, e.g., rubor (redness), swelling or edema, calor (heat), dolor (pain), and “functio laesa” (loss of function). When localized in skin or other tissue, biological/physiological effects comprise increased blood flow, enabling vascular leukocytes and plasma proteins to reach extravascular sites of injury, increasing local temperatures and generation of pain, tissue edema and extravascular pressure and a reduction in tissue perfusion. Other biological/physiological effects comprise organ and system dysfunction, such as cardiac dysfunction, adult respiratory distress syndrome, neurologic toxicity, renal and/or hepatic failure, and disseminated intravascular coagulation. Elevated levels of IFNγ, IL-6, TNFα, TGFbeta, IL-2, granulocyte macrophage-colony-stimulating factor (GM-CSF), IL-10, IL-8, IL-S, and/or fractalkine are implicated as predictive and/or causative of CRS or the propensity to elicit CRS upon T-cell stimulation.

The term a “pH-dependent” antibody refers to an antibody having a modified amino acid sequence that allows fir preferential or selective antigen binding at a certain pH. For example, an antibody can be engineered (e.g., by modifying the amino acid sequence) for pH-dependent binding. pH-dependent binding refers to an antibody's preference to bind an antigen at a given pH (or given pH range) as compared to a different pH (or pH range). In one embodiment, pH1-dependent antibodies preferentially or selectively bind to an antigen at an acidic pH (e.g., a pH at around 64 as compared to a physiological pH (e.g., a pH at around 7 or 74). An antibody sequence may be modified by, for example, substitution with one or more ionizable amino acid residues such as histidine, lysine, arginine, aspartic acid, and glutamic acid. Ionizable residues may be substituted into CDRs and/or the FRs. In some embodiments, 1-10 substitutions may be present per variant VH or VK. In some embodiments, 1-6 substitutions may be present per variant VH or VK. While the pH range of human blood is about 7.6-7.8, tumor cells have an extracellular pH of about 6.3-6.5 due at least in part to accumulation of metabolic acids that are inefficiently cleared because of poor tumor vascularization. Therefore, antibodies with antigen binding preference at a lower pH (e.g., pH ˜6) than a physiological pH is believed to provide selective and sustained cytotoxic activity at or around the tumor site, thereby potentially reducing or eliminating off-target effects and minimizing the CRS risk, as well as improving half-life and dosing. Therefore, in certain embodiments, the anti-CD3 antibodies and/or antigen-binding fragments described herein may have reduced likelihood of causing or may reduce the severity of potential CRS.

The tendency of antibodies to bind multiple targets is referred to as “polyspecificity,” which, with target-specific therapeutic antibodies, may be associated with negative clinical outcomes. Anti-CD3 antibodies and antigen-binding fragments of the present disclosure may exhibit reduced polyspecificity [e.g., as assessed by interaction with polyspecific reagent (PSR)]. Antibodies with reduced polyspecificity may be engineered from starting antibodies and antibody fragments by substituting various variable domain amino acid residues with residues having charged side chains. Residues may be substituted with amino acid residues having negatively charged side chains, e.g., Asp and Glu residues. Residues selected for substitution may be selected from those not predicted to specifically interact with CD3 amino acid residues bound by the anti-CD3 antibodies and antigen-binding fragments. Antibodies having high interaction with a PSR may be referred to as “polyspecific” antibodies and may be referred to as relatively “undevelopable” or relatively “non-developable”.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24, 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and bistidine; 6) acidic side chains: aspartate and glutarnate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, in some embodiments, a conservative replacement comprises any change having a positive value in the PAM250 log-likelihood matrix disclosed in Gonnet et al. (1992) Science 256, 1443 45. In some embodiments, a “moderately conservative” replacement comprises any change having a nonnegative value in a PAM250 log-likelihood matrix.

The term “nucleic acid” or “polynucleotide” refers to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis, recombinantly, or derived from a microorganism.

A “vector” is a compound or a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, viruses, and virus-like particles (VLPs). Thus, the term “vector” includes an autonomously replicating plasmid, a self-replicating RNA, or a viral particles. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

The term “host cell” refers to cells into which an exogenous nucleic acid sequence has been introduced, including the progeny of such cells. Host cells include transformants and transformed cells, which include the primary transformed cell and progeny derived therefrom without regard to the number of passages.

A “pharmaceutical formulation” refers to a preparation in such form as to permit the biological activity of an active ingredient contained therein, such as the anti-CD3 antibodies described herein, to be effective and which preferably contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In another embodiment, a pharmaceutical formulation comprises any of the anti-CD3 antibodies provided herein and at least one additional therapeutic agent.

An “effective amount” of an anti-CD3 antibody disclosed herein or a composition (e.g., pharmaceutical composition) comprising such antibody, is at least the minimum amount required to achieve the desired therapeutic or prophylactic result, e.g., a measurable improvement or prevention of a particular disorder, e.g., a cell proliferative disorder, e.g., cancer, preferably with minimal or no toxic or detrimental effects. An effective amount may vary according to inter alia disease state, age, sex, and weight of the patient, and the ability of the antibody (or antigen-binding fragment thereof) to elicit a desired response in the individual and, in some instances, by co-administering one or more additional therapeutic agents.

A “disorder” refers to any condition or disease that would benefit from treatment including, but not limited to, chronic and acute disorders or diseases including those pathological conditions which predispose a mammal to the disorder in question.

The terms “cell proliferative disorder” and “proliferative disorder” refer to disorders that are associated with some degree of abnormal cell proliferation. Cell proliferative disorders include cancer, e.g., a tumor.

“Tumor” as used herein refers to all neoplastic cell growth and proliferation, whether malignant or benign, and all pre-cancerous and cancerous cells and tissues.

“Cancer” refers to a physiological condition in mammals characterized by unregulated cell growth. Examples of cancer include, but are not limited to, carcinoma, lymphoma, blastoma, sarcoma, and leukemia or lymphoid malignancies; with more particular examples including squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer; rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NIHL; high grade small non-cleaved cell NHL; bulky disease NHL; mantle cell lymphoma; AIDS-related lymphoma; and Waldenström's Macroglobulinemia); chronic lymphocytic leukemia (CLL) acute lymphoblastic leukemia (ALL); hairy cell leukemia; chronic myeloblastic leukemia; and post-transplant lymphoproliferative disorder (PTLD), as well as abnormal vascular proliferation associated with phacomatoses, edema (such as that associated with brain tumors), Meigs' syndrome, brain, as well as head and neck cancer, and associated metastases. In certain embodiments, cancers that are amenable to treatment by antibodies of the disclosure include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (SLL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenström's macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenström's macroglobulinemia, Heavy chain diseases, a Heavy chain disease, γ Heavy chain disease, μ Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, AIX-positive large B-cell lymphoma, Plasmablastic lymphoma, Large B-cell lymphoma arising in HHV8-associated multicentric Castleman disease, Primary effusion lymphoma; B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

As used herein, “treatment” or “treat” or “treating” refer to clinical intervention in an attempt to alter the natural course of an individual being treated and can be performed either for prophylaxis or during the course of clinical pathology. Desirable effects of treatment include, but are not limited to, preventing occurrence or recurrence of disease, alleviation of symptoms, diminishment and/or reversal of any direct or indirect pathological consequences of the disease, preventing metastasis (e.g., in cancer), delaying progression of a disease/disorder/condition, decreasing the rate of disease progression, amelioration or palliation of the disease state, and remission or improved prognosis.

As used herein, the terms “prevent,” “preventing,” and “prevention” refer to the prevention or inhibition of the development or onset of a disorder or disease.

As used herein, the terms “ameliorate” and “alleviate” refer to a reduction or diminishment in the severity a condition or arty symptoms thereof.

As used herein, “delaying progression” of a disorder or disease means to defer, hinder, slow, retard, stabilize, and/or postpone development of the disease or disorder (e.g., a cell proliferative disorder, e.g., cancer). The delay can be of varying lengths of time, depending on the history of the disease and/or individual being treated.

“Detection” as used herein encompasses quantitative or qualitative detection.

### Anti-CD3 Antibody Variable Region Sequences

Provided herein are anti-CD3 antibodies and antigen-binding fragments that exhibit pH-dependent binding and optionally favorable developability profiles.

In some embodiments, the anti-CD3 antibody or antigen-binding fragment may comprise one or more CDR sequences that is/are (i) contained in any of Antibody Nos. A003-A013, (ii) contained in the variable domain sequences shown in Appendix Table A, and/or (iii) encoded in the variable domain-encoding nucleic acid sequences of any of Antibody Nos. A003-013 and/or shown in Appendix Table B. In some embodiments, the anti-CD3 antibody or antigen-binding fragment may comprise one or more CDR sequences shown in Appendix Tables D, F, H, K, M, and O.

In some embodiments, the anti-CD3 antibody or antigen-binding fragment may comprise the CDR-H3, the CDR-L1, and the CDR-L3 sequences of any of Antibody Nos. A003-A013. In certain embodiments, the anti-CD3 antibody or antigen-binding fragment may comprise the CDR-H3, the CDR-L1, and the CDR-L3 sequences of Antibody No. 6 or 5. In particular embodiments, the anti-CD3 antibody or antigen-binding fragment may comprise the CDR-H3, the CDR-L1, and the CDR-L3 sequences of Antibody No. 6.

In some embodiments, the anti-CD3 antibody or antigen-binding fragment may comprise the CDR-H1, the CDR-H2, the CDR-H3, the CDR-L1, the CDR-L2, and the CDR-L3 sequences of any of Antibody Nos. A003-A013. In certain embodiments, the anti-CD3 antibody or antigen-binding fragment may comprise the CDR-H1, the CDR-H2, the CDR-H3, the CDR-L1, the CDR-L2, and the CDR-L3 sequences of Antibody No. 6 or 5. In particular embodiments, the ani-CD3 antibody or antigen-binding fragment may comprise the CDR-H1, the CDR-H2, the CDR-H3, the CDR-L1, the CDR-L2, and the CDR-L3 sequences of Antibody No 6.

The anti-CD3 antibodies and antigen-binding fragments according to the present disclosure may comprise any appropriate FR sequences.

In some embodiments, the anti-CD3 antibody or antigen-binding fragment may comprise one or more heavy chain FR sequences encoded in the germline VH1-03 allele or another allele, and/or one or more heavy chain FR sequences comprising one, two, three, four, five, six, or more amino acid differences (substitutions, insertions, or deletions) relative to such germline-encoded sequences.

In some embodiments, the anti-CD3 antibody or antigen-binding fragment may comprise one or more light chain FR sequences encoded in the germline VK4-01 allele or another allele, and/or one or more light chain FR sequences comprising one, two, three, four, five, six, or more amino acid differences (substitutions, insertions, or deletions) relative to the germline-encoded sequences.

In some embodiments, the anti-CD3 antibody or antigen-binding fragment may comprise one or more FR sequences that is/are (i) contained in any of Antibody Nos. A003-A013, (ii) contained in the variable domain sequences shown in Appendix Table A, and/or (iii) encoded in the variable domain-encoding nucleic acid sequences of any of Antibody Nos. A003-013 and/or shown in Appendix Table B. In some embodiments, the anti-CD3 antibody or antigen-binding fragment may comprise one or more FR sequences shown in Appendix Tables C, E, G, I, J, L, N, and P.

In some embodiments, the anti-CD3 antibody or antigen-binding fragment may comprise the FR-H1, the FR-H2, the FR-H3, the FR-H4, the FR-L1, the FR-L2, the FR-L3, and the FR-L4 sequences of any of Antibody Nos. A003-A013. In certain embodiments, the anti-CD3 antibody or antigen-binding fragment may comprise the FR-H1, the FR-H2, the FR-13, the FR-H4, the FR-L1, the FR-L2, the FR-L3, and the FR-L4 sequences of Antibody No. 6 or 5. In particular embodiments, the anti-CD3 antibody or antigen-binding fragment may comprise the FR-H1, the FR-H2, the FR-H3, the FR-H4, the FR-L1, the FR-L2, the FR-L3, and the FR-L4 sequences of Antibody No. 6.

In some embodiments, the anti-CD3 antibody or antigen-binding fragment may comprise a VH polypeptide comprising an amino acid sequence at least 80%, at least 85%, at least 90%, at least 92%, at least 94%, at least 95%, at least 96%, at least 97%, at least 98%, at least 99%, or 100% identical to: ii) the VH polypeptide sequence of any of Antibody Nos. A003-A013; (ii) the VH polypeptide sequence encoded by the V1H-encoding sequences of any of Antibody Nos. A003-A013, (iii) any of the VH polypeptide sequences shown in Appendix Table A; and/or (iv) the VH polypeptide sequence encoded by the VH-encoding sequences shown in Appendix Table B.

In some embodiments, the anti-CD3 antibody or antigen-binding fragment may comprise a VL polypeptide comprising an amino acid sequence at least 80%, at least 85%, at least 90%, at least 92%, at least 94%, at least 95%, at least 96%, at least 97%, at least 98%, at least 99%, or 100% identical to: (i) the VL polypeptide sequence of any of Antibody Nos A003-A013; (ii) the VL polypeptide sequence encoded by the VL-encoding sequences of any of Antibody Nos. A003-A013; (iii) any of the VL polypeptide sequences shown in Appendix Table A; and/or (iv) the VL polypeptide sequence encoded by the VL-encoding sequences shown in Appendix Table B.

In certain embodiments, percent identity may be measured by any well-known algorithm of sequence identity, such as FASTA, BLAST or GAP.

In particular embodiments, the anti-CD3 antibody or antigen-binding fragment may comprise: (A) a VH polypeptide comprising (i) the CDR-H1, the CDR-H2, and the CFR-H3 sequences of SEQ ID NOS: 612, 614, and 616, respectively; and (ii) an amino acid sequence at least 90% identical to SEQ ID NO: 610; and (B) a VL polypeptide comprising (i) the CDR-L1, the CDR-L2, and the CFR-L3 sequences of SEQ ID NOS: 622, 624, and 626, respectively; and (ii) an amino acid sequence at least 90% identical to SEQ ID NO: 620.

In particular embodiments, the anti-CD3 antibody or antigen-binding fragment may comprise: (A) a VH polypeptide comprising (i) the CDR-H1, the CDR-H2, and the CFR-H3 sequences of SEQ ID NOS: 512, 514, and 516, respectively; and (ii) an amino acid sequence at least 90% identical to SEQ ID NO: 510; and (B) a VL polypeptide comprising (i) the CDR-L1, the CDR-L2, and the CFR-L3 sequences of SEQ ID NOS: 522, 524, and 526, respectively; and (ii) an amino acid sequence at least 90% identical to SEQ ID NO: 520.

In certain embodiments, the anti-CD3 antibody or antigen-binding fragment may comprise a VH polypeptide and a VL polypeptide comprising any of the VH and VL amino acid sequence combinations shown in Appendix Table A.

In particular embodiments, the anti-CD3 antibody or antigen-binding fragment may comprise: (A) a VH polypeptide comprising SEQ ID NO: 610; and (B) a VL polypeptide comprising SEQ ID NO: 620.

In particular embodiments, the anti-CD3 antibody or antigen-binding fragment may comprise: (A) a VH polypeptide comprising SEQ ID NO: 510; and (B) a VL polypeptide comprising SEQ ID NO: 520.

### Antibody Constant Domains and Regions

The anti-CD3 antibodies and antigen-binding fragments according to the present disclosure may or may not comprise one or more immunoglobulin (Ig) constant domains (e.g., CH1, CH2, and/or CH3), one or more Fc regions (e.g., part of hinge, CH2, and CH3), and/or one or more constant regions (e.g., one or more sets of CH1, hinge, CH2, and CH3) or one or more parts thereof (e.g., one or more sets of CH1 and part of hinge). Such constant domains, Fc regions, and/or constant regions or parts thereof may individually be or may individually be derived from any Ig isotypes (e.g., human IgG, IgA, IgE, IgM, or IgD) and subclasses (e.g., human IgG1, IgG2, IgG3 or IgG4) and variants thereof and may optionally comprise any of the modifications described herein.

In some embodiments, one or more amino acid modifications may be introduced into the Fc region of an anti-CD3 antibody of the disclosure, thereby generating an Fc region variant (see, e.g., US 2012/0251531). An Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the disclosure contemplates an anti-CD3 antibody variant that possesses some but not all effector functions, which make it a desirable candidate for applications in which the half-life of an antibody in vivo is important yet certain effector functions (such as complement and ADCC) are unnecessary or deleterious. In vitro and/or in vivo cytotoxicity assays can be conducted to confirm reduction/depletion of CDC and/or ADCC activities. For example, Fc receptor (FcR) binding assays can be conducted to ensure that an antibody lacks FcγR binding (hence likely lacking ADCC activity) but retains FcRn binding ability. The primary cells for mediating ADCC (e.g. NK cells), express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see, Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (Cell Technology, Inc. Mountain View, Calif.); and CYTOTOX 96; non-radioactive cytotoxicity assay (Promega, Madison, Wis.)), Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that an antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol Methods 202:163 (1996). Cragg, M. S. et al. Blood. 101:1045-1052 (2003); and Cragg, M. S, and M. J. Glennie Blood. 103:2738-2743 (2004)). FcRn binding and in vivo clearance/half-lite determinations can also be performed using methods known in the art (see, e.g., Petkova, S. B. et al. Int'l. Imunol 18(12): 1759-1769 (2006)).

In certain embodiments, antibodies with reduced effector function include those with substitution of one or more of Fc region residues 238, 265, 269, 270, 297, 327 and 329 (U.S. Pat. Nos. 6,737,056 and 8,219,149). In some embodiments, Fe mutants include Fc mutants with substitutions at two or more of amino acid positions 265, 269, 270, 297 and 327, including the so-called “DANA” Fe mutant with substitution of residues 265 and 297 to alanine (U.S. Pat. Nos. 7,332,581 and 8,219,149).

In some embodiments, anti-CD3 antibodies and/or antigen binding fragments as described herein may comprise silent Fc regions (e.g., removing the Fc completely or modifying the Fe region to reduce or eliminate effector function), and/or may comprise a masking agent (e.g., a polypeptide mask that is positioned (e.g., attached via a cleavable linker) such that it reduces or inhibits the ability of the antibody or antigen-binding fragment to induce an effector function-inducing molecule, such as a complement (e.g., C1q)).

### Antigen-Binding Fragments and Multispecific Antibodies

Anti-CD3 antigen-binding fragments according to the present disclosure may take any appropriate formats including but not limited to any of the formats described herein (e.g., Fab, scFv, etc), as long as such modifications preserve pH-dependent CD3 binding and do not substantially reduce the ability of the antibody to bind to CD3.

In certain embodiments, the anti-CD3 antibodies and/or antigen-binding fragments thereof as described herein may comprise or may be comprised in a multispecific antibody or antibody fragment, which may comprise at least two different variable regions, wherein each variable region is capable of specifically binding to a separate antigen or to a different epitope on the same antigen in particular, a bispecific antibody that has binding specificity for a second antigen. In some embodiments, the binding specificities are to two different epitopes of CD3 (e.g., CD3ε or CD3γ). In other embodiments, one of the binding specificities is for CD3 (e.g., CD3ε or CD3γ) and the other is for a different biological molecule (e.g., a cell surface antigen, e.g., a tumor antigen).

Multispecific antibodies comprising at least one anti-CD3 antibody and/or antigen-binding fragment disclosed herein may be prepared according to a variety of techniques including, but are not limited to, recombinant co-expression of two immunoglobulin heavy chain-light chain pairs having different specificities (see, Milstein and Cuello, Nature 305: 537 (1983)), WO 93/08829, and Traunecker et al., EMBO J. 10: 3655 (1991)), “knob-in-hole” engineering (see, e.g., U.S. Pat. No. 5,731,168); immunoglobulin crossover (also known as Fab domain exchange or CrossMab format) technology (see, e.g., WO2009/080253; Schaefer et al., Proc. Natl. Acad. Sci. USA, 108:11187-11192 (2011)), engineering electrostatic steering, effects for making antibody Fc-heterodimeric molecules (WO 2009/089004A1); cross-linking two or more antibodies or fragments (vee, e.g., U.S. Pat. No. 4,676,980, and Brennan et al., Science, 229: 81 (1985)); leucine zippers (see, e.g., Kostelny et al., J. Immunol, 148(5):1547-15S3 (1992)), “diabody” technology (see, e.g., Hollinger et al., Proc. Natl. Acad. Sci. USA, 90:6444-6448 (1993)); single-chain Fv (sFv) dimers (see, e.g. Gruber et al., J. Immunol, 152:5368 (1994)); and trispecific antibodies as described, e.g., in Tutt et al. J. Immunol 147: 60 (1991).

A variety of multispecific antibody formats may be used in the context of an antigen-binding fragment of anti-CD3 antibody described herein. Non-limiting examples of multispecific and bispecific formats include, e.g., Fab-Fc-scFv (“bottle-opener”) (XENCOR), Mab-scFv (XENCOR), Mab-Fv (XENCOR), Dual scFv (XENCOR), central Fv (XENCOR), central scFv(XENCOR), one-arm central scFv (XENCOR), Fab-Fab (XENCOR), Fab-Fv (XENCOR), mAb-Fv (XENCOR), mAb-Fab (XENCOR), DART (MACROGENICS), BiTE (AMGEN/MICROMET), KiTE, common light chain-IgG (Genentech), TandAb (SFFIMED) Cross-Mab (ROCHE), SEED (EMD SERONO), BEAT (GLENMARK), TrioMab (TRION PHARMA/FRESNIUS BIOTECH), DuetMab (MEDIMMUNE), and others, as disclosed, e.g., in (WO2021067404; WO2022150787; WO2022150785; WO 95:09917; WO 2008/119566; WO 2008/119567; WO2011/121110; WO 2010/037835; WO 2007/042261; WO 2007/110205; WO 2011/12111); WO 2012/055961; WO 2012/16067; WO 2016/086189: WO 2016/182751, WO 2015/006749; WO 2014/049003; WO 2013/177101; WO 2015/128509; U.S. Pat. No. 7,951,917; US 2009/0252729; US 2014/0348839; U.S. Pat. No. 7,183,076; Mazor et al., Mabs, Vol. 7, pages 377-389 (2015); Muda et al., Protein Engineering, Design, & Selection, Vol. 24, pages 447-454 (2011); and Del Bano et al., Antibodies, Vol. 5, pages 1-23 (2016). In some embodiments, the anti-CD3 scFv fragments described herein may comprise one or more variable regions of a multispecific (e.g., bispecific) antibody.

In some embodiments, a multispecific antibody or antibody fragment may comprise one or more engineered, variant constant domains which facilitate efficient polypeptide heterodimerization (e.g., a first heavy chain and a second heavy chain that is different from the first heavy chain) for bispecific antibody formation.

In certain embodiments, a multispecific antibody or antibody fragment may comprise at least one CLκ-preferring variant CH1 domain and/or a CLλ-preferring variant CH1 domain. A CLκ-preferring variant CH1 domain preferentially pairs with a CLκ domain rather than a non-CLκ domain (such as a CLλ domain). A CLλ-preferring variant CH1 domain preferentially pairs with a CLλ domain rather than a non-CLλ domain (such as a CLκ domain). In particular embodiment, such a CLκ-preferring variant CH1 domain and/or a CLκ-preferring variant CH1 domain may be selected from those described in WO2021067404.

In certain embodiments, a multispecific antibody or antibody fragment may comprise at least one pair of a CH1 domain and a CL domain which preferentially pair with each other. In a preferentially-pairing pair of CH1 and CL domains: the CH1 domain prefers to pair with the CL domain rather than another given CL domain such as a wildtype CL domain; and/or the CL domain prefers to pair with the CH1 domain rather than another given CH1 domain such as a wildtype CH1 domain. One or both of the CH1 domain and the CL domain may be variant domain(s). In particular embodiments, such a preferentially-pairing pair of a CH1 domain and a CL domain may be selected from the pairs described in WO2022150787.

In certain embodiments, a multispecific antibody or antibody fragment may comprise at least one pair of a first CH3 domain and a second CH3 domain that differs from the first CH3 which preferentially pair (i.e., form a heterodimer) with each other. In such a preferentially-pairing pair, the first CH3 domain prefers to pair with the second CH3 domain rather than another first CH3 domain; and/or the second CH3 domain prefers to pair with the first CH3 domain rather than with another second CH3 domain. One or both of the CH3 domains may be variant domain(s). In particular embodiments, such a preferentially-pairing pair of a first and second CH3 domains may be selected from the pairs described in WO2022150785.

### Exemplary Technical Advantages

Exemplary technical advantages provided by some of the embodiments according to the present disclosure are listed below. It is noted that technical advantages possessed by such embodiments may not be limited to the advantages specifically listed below. It is also noted that embodiments other than the embodiments specifically mentioned below may also have one or more of the advantages listed below.

### Binding to Cynomolgus CD3

As shown in Table 2B, while the parent pH-dependent antibody, Antibody No. 2, does not bind to cynomolgus CD3, novel pH-dependent antibodies, Antibody Nos. 3-10 and 12, bind to cynomolgus CD3.

Non-human primates such as cynomolguses are often ideal species for establishing pharmacokinetics and collecting efficacy data of antibodies during pre-clinical studies due to the similarity to humans. Without wishing to be bound by theory, the ability to bind to cynomolgus CD3 is a technical advantage because it allows for various in vitro studies using cynomolgus CD3, which would help design such pre-clinical studies in cynomolguses.

In some embodiments, the anti-CD3 antibody or antigen-binding fragment according to the present disclosure may comprise one or more CDRs of any of Antibody Nos. 3-10 and 12. In certain embodiments, the anti-CD3 antibody or antigen-binding fragment according to the present disclosure may comprise the combination of six CDRs of any of Antibody Nos. 3-10 and 12. In particular embodiments, the anti-CD3 antibody or antigen-binding fragment according to the present disclosure may comprise the combination of six CDRs of Antibody No. 6 or 5.

### Improved Binding to Human CD3 at an Acidic pH

As shown in Table 2A, novel pH-dependent antibodies, Antibody Nos. 3-12, bind to human CD3 at pH 6.0 with higher affinity than the parent pH-dependent antibody, Antibody No. 2, when measured via BLI using a ForteBio OCTET® system.

As described above, tumor cells typically have an extracellular pH of about 6.3-6.5 due at least in part to accumulation of metabolic acids that are inefficiently cleared because of poor tumor vascularization. Therefore, without wishing to be bound by theory, antibodies having higher affinity at an acidic pH such as pH 6.0 would exhibit more potent effect (e.g., higher cytotoxic activity) at or around the tumor site. Furthermore, without wishing to be bound by theory, this would allow for use of a reduced dose and therefore the toxicity would be minimized.

In some embodiments, the anti-CD3 antibody or antigen-binding fragment according to the present disclosure may comprise one or more CDRs of any of Antibody Nos. 3-12. In certain embodiments, the anti-CD3 antibody or antigen-binding fragment according to the present disclosure may comprise the combination of six CDRs of any of Antibody Nos. 3-12. In particular embodiments, the anti-CD3 antibody or antigen-binding fragment according to the present disclosure may comprise the combination of six CDRs of Antibody No. 6 or 5.

### Improved Binding to Human CD3-Expressing Cells at an Acidic pH

As shown in Table 3, novel p t-dependent antibodies, Antibody Nos. 3 and 5-11, bind to human CD3-expressing cells (CD3+Jurkat cells) at pH 6.0 more than the parent pH-dependent antibody, Antibody No. 2, based on the NCB values obtained from flow cytometric analyses.

As described above, tumor cells typically have an extracellular pH of about 6.3-6.5, which is lower than the physiological pH (around pH 7.4). Therefore, without wishing to be bound by theory, antibodies having better binding to cells at art acidic pH such as pH 6.0 would exhibit more potent effects (e.g. higher cytotoxic activity) at or around the tumor site. For example, such antibodies may provide better binding to T cells while also binding to a tumor cell, thereby more potent cytotoxic effects may be exerted by the T cells against the tumor cell. Furthermore, without wishing to be bound by theory, this would allow for use of a reduced dose and therefore the toxicity would be minimized.

In some embodiments, the anti-CD3 antibody or antigen-binding fragment according to the present disclosure may comprise one or more CDRs of any of Antibody Nos. 3-12. In certain embodiments, the anti-CD3 antibody or antigen-binding fragment according to the present disclosure may comprise the combination of six CDRs of any of Antibody Nos. 3-12. In particular embodiments, the anti-CD3 antibody or antigen-binding fragment according to the present disclosure may comprise the combination of six CDRs of Antibody No. 6 or 5.

### Improved Binding to Cynomolgus CD3-Expressing Cells at an Acidic pH

As shown in Table 3, novel pH-dependent antibodies, Antibody Nos. 3 and 5-11, bind to cynomolgus CD3-expressing cells (HSC-F cells) at pH 6.0 more than the parent pH-dependent antibody, Antibody No. 2, based on the NCB values obtained from flow cytometric analyses.

As described above, non-human primates such as cynomolguses are often ideal species for establishing pharmacokinetics and collecting efficacy data of antibodies during pre-clinical studies due to the similarity to humans. Without wishing to be bound by theory, improved binding to cynomolgus CD3-expressing cells is a technical advantage, because it increases the chance of success in such pre-clinical studies, which is critical for bringing antibodies to the clinical stage.

In some embodiments, the anti-CD3 antibody or antigen-binding fragment according to the present disclosure may comprise one or more CDRs of any of Antibody Nos. 3 and 5-11. In certain embodiments, the anti-CD3 antibody or antigen-binding fragment according to the present disclosure may comprise the combination of six CDR3 of any of Antibody Nos. 3 and 5-11. In particular embodiments, the anti-CD3 antibody or antigen-binding fragment according to the present disclosure may comprise the combination of six CDRs of Antibody No. 6 or 5.

### Further Modifications

### Sequence Modifications

The present disclosure, also contemplates modification of anti-CD3 antibodies disclosed herein, such as one or more amino acid modifications (substitutions, insertions, or deletions) in the VH and/or VL polypeptides and/or constant regions sequences relative to the corresponding sequences of any of the antibodies described above. Once obtained, such derivative antibodies and/or antigen-binding fragments can be tested for one or more desired properties such as improved binding specificity, increased binding affinity, improved pH-dependent antigen binding, improved developability, etc.

In some embodiments, such modifications may be in the FR and/or CDR regions.

In some embodiments, the one or more substitutions, insertions, or deletions may occur within one or more CDRs of the anti-CD3 antibodies described herein, so long as such modifications preserve pH sensitivity and do not substantially reduce the ability of the antibody to bind its antigen. For example, conservative alterations (e.g., conservative amino acid substitutions as provided herein) that do not substantially reduce binding affinity may be made in CDRs. Such alterations may, for example, be outside of antigen contacting residues in the CDRs. In certain embodiments each of the six CDRs may contain no more than one, two or three amino acid substitutions. In certain embodiments the CDR-H1, the CDR-H2, and/or the CDR-L2 may contain no more than one, two or three amino acid substitutions relative to any of Antibody Nos. 3-13, preferably relative to Antibody No. 6 or 5. In particular embodiments, the CDR-H3, the CDR-L1, and CDR-L3 may not comprise arty substitutions, insertions, or deletions relative to the CDR-H3, the CDR-L1, and the CDR-L3 of any of Antibody Nos. 3-13, preferably of Antibody No. 6 or 5.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of an antibody molecule include fusion to the N- or C-terminus of the antibody to an enzyme (e.g. for ADEPT) or a polypeptide which increases serum half-life of the antibody.

In addition to substitution of one or more CDR residues, omission of one or more CDRs is also possible. Antibodies have been described in which one or two CDRs can be dispensed to alter binding in the scientific literature Padlan et al. (1995 FASEB J. 9:133-139) analyzed contact regions between antibodies and their antigens, based on published crystal structures, and concluded that only about one fifth to one third of CDR residues actually contact their associated antigen. Padlan also found many antibodies in which one or two CDRs had zero amino acids in contact with an antigen (see also, Valdos et al. 2002 J Mol Biol 320:415-428), CDR residues not contacting an antigen can be identified based on previous studies (for example residues 1160-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

### Modification Via Conjugation

In some embodiments, anti-CD3 antibodies and/or antigen-binding fragments thereof as described herein are conjugated to a moiety or agent, thereby forming an immunoconjugate.

In certain embodiments, the anti-CD3 antibodies according to the present disclosure may be further modified to contain additional nonproteinaceous moieties that are known in the art and are readily available. Moieties suitable for derivatization of an antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, labeled anti-CD3 antibodies are provided. Anti-CD3 antibodies and/or antigen-binding fragments thereof as described herein may include a label or moiety that is detected directly (such as fluorescent, chromophoric, electron-dense, chemiluminescent, and radioactive labels or indirectly (such as enzymes or ligands). Non-limiting exemplary labels include, radioisotopes such as 32P, 14C, 125I, 3H, and 131I; fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase; hetetrocyclic oxidases such as uricase and xanthine oxidase, coupled with an enzyme that employs hydrogen peroxide to oxidize a dye precursor such as HRP, lactoperoxidase, or microperoxidase; biotin/avidin; spin labels; bacteriophage labels; stable free radicals; and the like.

In certain embodiments, the anti-CD3 antibody or antigen-binding fragment of the present disclosure may be, may comprise, or may be comprised in an antibody-drug conjugate (ADC). The ADC may comprise: (a) any anti-CD3 antibody or antigen-binding fragment described herein; and (b) a drug conjugated thereto. In certain embodiments, the drug may be for example, but not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug. In some cases, the drug may be selected from the group consisting of doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydoxyellipticine, thiocolchicine, methotrexate, triptolide, taltobulin, lactacystin, dolastatin, auristatin, monomethyl auristatin E (MMAE), monoethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, and a derivative of any one of the foregoing.

### Glycosylation

In some embodiments, anti-CD3 antibodies and/or antigen-binding fragments thereof as described herein may be altered to increase or decrease the extent to which the antibody is glycosylated. Addition or deletion of glycosylation sites to an anti-CD3 antibody of the disclosure may be conveniently accomplished by altering the amino acid sequence such that one or more glycosylation sites is created or removed. In certain embodiments, addition or deletion of glycosylation sites may not be limited to the constant region of an anti-CD3 antibody or antigen-binding fragment.

### Antibody Characterization and Further Screening

In some embodiments, the anti-CD3 antibodies and/or antigen-binding fragments described herein may be characterized for their physical/chemical properties and/or biological activities by various assays known in the art. In some embodiments, variants of any of the anti-CD3 antibodies and antigen-binding fragments described herein thereof may be identified based on, screened based on, selected based on, or characterized for their physical/chemical properties and/or biological activities by various assays known in the art. In some embodiments, a multispecific antibody or antibody fragment comprising an anti-CD3 antibody or antigen-binding fragment described herein may be designed and screened based on, selected based on, or characterized for their physical/chemical properties and/or biological activities by various assays known in the art.

In certain embodiments, such assays may include SPR, BLI, ELISA, Western blot, flow cytometry, etc.

In certain embodiments, such assays may include competition assays, which in some cases may be used to identify an antibody that competes with a given anti-CD3 antibody of the disclosure for binding to CD3. In an exemplary competition assay, immobilized CD3 is incubated in a solution comprising a first labeled antibody that binds to CD3 and a second unlabeled antibody that is being tested for its ability to compete with the first antibody for binding to CD3. The second antibody may be present in a hybridoma supernatant. As a control, immobilized CD3 is incubated in a solution comprising the first labeled antibody but not the second unlabeled antibody. After incubation under conditions permissive for binding of the first antibody to CD3, excess unbound antibody is removed, and the amount of label associated with immobilized CD3 is measured. If the amount of label associated with immobilized CD3 is substantially reduced in the test sample relative to the control sample, then that indicates that the second antibody is competing with the first antibody for binding to CD3. See, e.g., Harlow and Lane (1988) Antibodies: A Laboratory Manual, Ch. 14 (Cold Spring Harbor Laboratory, Cold Spring Harbor, N Y.).

Anti-CD3 antibodies and/or antigen-binding fragments thereof possessing biological activity may be identified using standard approaches. Biological activity may include, e.g., binding to CD3 on the surface of a T cell either in vivo, in vitro, or ex vivo. In the case of a multispecific anti-CD3 antibody (such as a bispecific antibody with one arm that binds to CD3 and another arm that binds to a different target, e.g., a cell surface antigen, e.g., a tumor antigen), biological activity may also include effector cell activation (such as CD8+ and/or CD4+ T cell) activation), effector cell population expansion (i.e., an increase in T cell count), target cell population reduction (i.e., a decrease in the population of cells expressing the second biological molecule on their cell surfaces), and/or target cell killing,

### Evaluation Based on Target Binding

In some embodiments, an anti-CD3 antibody or antigen-binding fragment as described herein may exhibit a level of binding to CD3 in a preferred range at an approximate pH of interest.

In certain embodiments, antigen binding may be determined based on the K_{D} value obtained by surface plasmon resonance (SPR), optionally using a BIACORE™ system. In certain embodiments, the KU by SPR may be determined as described in Examples.

In certain embodiments, an anti-CD3 antibody or antigen-binding fragment as described herein may have a K_{D} value for human CD3 of between about 0.5 nM and about 50 nM at about pH 6.0. In certain embodiments, the K_{D} by SPR for human CD3 at pH 6.0 may be between about 0.5 nM and about 20 nM, between about 0.5 nM and about 20 nM, between about 1 nM and about 10 nM, or between about 5 nM and about 10 nM. In certain embodiments, an anti-CD3 antibody or antigen-binding fragment as described herein may have a Kₙ value by SPR for human CD3 at about pH 7.4 of about 1 nM or higher. In certain embodiments, the K_{D} by SPR for human CD3 at pH 7.4 may about 10 nM or higher, or 100 nM or higher, 1000 nM or higher, or 10000 nM or higher. In particular embodiments, no binding is observed at pH 7.4.

In certain embodiments, antigen binding may be determined based on the K_{D} value obtained by bio-layer interferometry (BLI), optionally using an OCTET® system. In certain embodiments, the K_{D} by BLI may be determined as described in Examples.

In certain embodiments, an anti-CD3 antibody or antigen-binding fragment as described herein may have a K_{D} value by BLI for human CD3 at about pH 6.0 of between about 0.5 nM and about 20 nM. In certain embodiments, the K_{D} by BLI for human CD3 at pH 6.0 may be between about 1 nM and about 10 nM, or between about 5 nM and about 7 nM. In certain embodiments, an anti-CD3 antibody or antigen-binding fragment as described herein may have a K) value by BLI for human CD3 at about pH 7.4 of between about 5 nM or higher. In certain embodiments, the K_{D} by BLI for human CD3 at pH 7.4 may about 10 nM or higher.

In certain embodiments, an anti-CD3 antibody or antigen-binding fragment as described herein may have a K_{D} value by BLI for non-human primate (e.g., cynomolgus or rhesus) CD3 at about pH 6.0 of between about 0.5 nM and about 20 nM. In certain embodiments, the K_{D} by BLI for non-human primate CD3 at pH 6.0 may be between about 1 nM and about 10 nM, or between about 5 nM and about 8 nM. In certain embodiments, an anti-CD3 antibody or antigen-binding fragment as described herein may have a K_{D} value by BLA for non-human primate CD3 at about pH 7.4 of between about 5 nM or higher. In certain embodiments, the K_{D} by BLI for non-human primate CD3 at pH 7.4 may about 20 nM or higher.

Without wishing to be bound by theory, an antibody with higher affinity (i.e., low K_{D} value) may induce more potent cytokine release by and/or activation of CD3-expressing cells upon antigen binding, and in some cases, the potent cytokine release and/or activation may help reduce the dose (e.g., an effective amount) of an anti-CD3 antibody or antigen-binding fragment needed for therapy.

In some embodiments, an anti-CD3 antibody or antigen-binding fragment as described herein may exhibit higher binding to CD3-expressing cells at an approximate pH of interest.

In certain embodiments, pH-dependent binding to CD3-expressing cells may be determined by fluorescence-activated cell sorting (FACS), optionally based on median fluorescence intensity (MFI) values and/or normal cell binding (NCB) values calculated using MFI values. In certain embodiments, the MFI and/or NCB values may be determined as described in Examples, wherein cells are incubated with a test antibody followed by a secondary antibody which binds to the test antibody (negative controls including incubation with no test antibody followed by a secondary antibody). In some cases, NCB value is calculated as {(Sample MFI)−(Secondary Only MFI)}/(Secondary Only MFI).

In certain embodiments, an anti-CD3 antibody or antigen-binding fragment as described may have a NCB value for binding to a human CD3-expressing cell at about pH 6.0 that is at least ×1.2 greater than the NCB value for binding to a human CD3-expressing cell at about pH 7.4. In certain embodiments, the NCB value at about pH 6.0 may be at least ×1.3, at least ×1.4, at least ×1.5, at least ×2, at least ×3, at least ×4, at least ×5, at least ×10, at least ×20, at least ×50, at least ×100, at least ×10³, at least ×10⁴, at least ×10⁵, at least ×10⁶, at least ×10⁷, at least ×10⁸, or at least ×10⁹ greater than the NCB value at about pH 7.4. In particular cases, the NCB value at about pH 6.0 may be at least ×3, at least ×4, or at least ×5 greater than the NCB value at about pH 7.4 Optionally, the human CD3-expressing cell may be a CD3-expressing Jurkat cell.

In some embodiments, an anti-CD3 antibody or antigen-binding fragment as described herein may exhibit improved binding to CD3-expressing cells (e.g., human CD3-expressing cells) at an acidic pH (e.g., about pH 6.0) relative to a known anti-CD3 antibody.

In certain embodiments, unproved binding to CD3-expressing cells may be determined by FACS, optionally based on MFI values and/or NCB values calculated using MFI values. In certain embodiments, the MFL and/or NCB values may be determined as described in Examples. In some cases, NCB value may be calculated as described above.

Without wishing to be bound by theory, an antibody with higher or improved binding (i.e., higher NCB values) to human CD3-expressing cells (e.g., T cells) at an acidic pH (e.g., about pH 6.0) may induce more potent cytokine release by and/or activation of the CD3-expressing cells upon antigen binding in an acidic environment, and in some cases, the potent cytokine release and/or activation may help reduce the dose (e.g., an effective amount) of an anti-CD3 antibody or antigen-binding fragment needed for therapy.

### Evaluation Based on Other Developability Parameters

In some embodiments, anti-CD3 antibodies and/or antigen-binding fragments including multispecific antibodies as provided herein and variants thereof (e.g., a multispecific antibody designed to comprise a second specificity of interest) may be identified based on, screened based on, selected based on, or characterized for developability.

In certain embodiments, an anti-CD3 antibody or antigen-binding fragment may display a favorable developability profile. The developability profile for anti-CD3 antibodies may be obtained by performing one or more of a PSR assay; an SCP assay; AC-SINS; an ELISA; a DSF assay; a Tm assay; a HIC assay; a CIC assay; or combinations thereof.

In some embodiments, an anti-CD3 antibody or antigen-binding fragment as described herein may exhibit reduced polyspecificity (tendency to bind to multiple molecules or epitopes). In certain embodiments, polyspecificity may be determined based on the poly-specificity reagent (PSR) score obtained by a PSR assay. In certain embodiments, PSR scores may be determined as described in Examples. In some embodiments, an anti-CD3 antibody or antigen-binding fragment as described herein display a PSR score of between about 0.0 and about 0.45. In some embodiments, the PSR score is between about 0.0 and about 0.4. In some embodiments, the PSR is between about 0.0 and about 0.35. In some embodiments, the PSR is between about 0.0 and about 0.3. In some embodiments, the PSR is between about 0.0 and about 0.25. In some embodiments, the PSR is between about 0.0 and about 0.2. In some embodiments, the PSR is between about 0.0 and about 0.15. In some embodiments, the PSR is between about 0.0 and about 0.1. In some embodiments, a score of 0.0-0.1 is “clean PSR”. In some embodiments, a score of 0.1 to 0.33 is “low PSR”. In some embodiments, a score of 0.33 to 0.66 is “medium PSR”. In some embodiments, a score of 0.66-1.00 is “high PSR”. In some embodiments, a high PSR score is indicative of decreased (or poor) developability. Generally, the lower the PSR score the more favorable the developability of the antibody.

In some embodiments, an anti-CD3 antibody or antigen-binding fragment as described herein may exhibit reduced hydrophobicity. In certain embodiments, hydrophobicity may be determined based on the retention time observed during HIC. In certain embodiments, HIC may be performed and retention times may be obtained as described in Examples. In some embodiments, anti-CD3 antibodies or antigen-binding fragment thereof as described herein display an HIC score of less than about 10.5 minutes (a clean to low HIC score). In some embodiments, art HIC score is between about 10.5 minutes and 11.5 minutes (a medium HIC score). In some embodiments, an HIC score is greater than about 11.5 minutes (a high HIC score). Generally, the lower the HIC score the more favorable the developability of the antibody.

In some embodiments, an anti-CD3 antibody or antigen-binding fragment as described herein may exhibit reduced tendency for self-interaction. In certain embodiments, tendency for self-interaction may be determined based on Δλmax values observed during affinity-capture self-interaction nanoparticle spectroscopy (AC-SINS). In certain embodiments, AC-SINS may be per formed and Δλmax values may be obtained as described in Examples. In some embodiments, an anti-CD3 antibody or antigen-binding fragment thereof as described herein may display Δλmax of between about 0.0 nm and about 15.0 nm. In some embodiments, the Δλmax may be between about 0.0 nm and about 10.0 nm. In some embodiments, the Amax may be between about 0.0 nm and about 7.5 nm. In some embodiments, the Δλmax may be between about 0.0 nm and about 5.0 nm. In some embodiments, the Δλmax may be between about 0.0 nm and about 3.0 nm. In some embodiments, the Amax may be between about 0.0 nm and about 2.0 nm. In some embodiments, the Δλmax may be between about 0.0 nm and about 1.0 nm. In some embodiments, 0.0 nm≤Δλmax<5.0 nm is considered as “low self-interaction”. In some embodiments, 5.0 nm≤Δλmax<20.0 nm is considered as ‘medium self-interaction’. In some embodiments, 10.0 nm≤Δλmax is considered as “high self-interaction”. Generally, the lower the tendency for self-interaction the more favorable the developability of the antibody.

In some embodiments, an anti-CD3 antibody or antigen-binding fragment as described herein may exhibit reduced viscosity. In some embodiments, viscosity may be determined based on diffusion interaction parameter (kD) values observed during dynamic light scattering (DLS). In some embodiments, DLS may be performed and kD values may be obtained as described in Examples, e.g., using 10 mM histidine buffer (pH about 6). In some embodiments, an anti-CD3 antibodies or antigen-binding fragment as described herein may display a kD of about 5 mL/g or higher. In some embodiments, the kD may be about 10 mL/g or higher. In some embodiments, the kD may be about 15 mL/g or higher. In some embodiments, the kD may be about 20 mL/g or higher. In some embodiments, the kD may be about 25 mL/g or higher. In some embodiments, Δλmax≤20 mL/g may be considered to be associated with high viscosity or high opalescence, Generally, the lower the viscosity the more favorable the developability of the antibody.

In some embodiments, an anti-CD3 antibody or antigen-binding fragment as described herein may exhibit reduced chance of heavy-light chain mispairing (including failure to pair). In some embodiments, heavy-light chain mispairing may be determined based on the presence of a heavy chain peak (indicating a heavy chain which did not successfully pair with a light chain) and/or a light chain peak (indicating a light chain which did not successfully pair with a heavy chain) observed during liquid chromatography-mass spectrometry (LC-MS). In some embodiments, LC-MS may be performed as described in Examples. In some embodiments, an anti-CD3 antibody or antigen-binding fragment as described herein may display no heavy chain peak or light chain peak. Generally, the smaller the heavy chain peak and light chain peak the more favorable the developability of the antibody.

In some embodiments, an anti-CD3 antibody or antigen-binding fragment as described herein may exhibit reduced propensity to aggregate. In some embodiments, propensity to aggregate may be determined based on % monomer values (i.e., % of antibody species (e.g., IgG or Fab) that are existing in its full size without aggregation or multimerization, among proteins from antibody production and optionally purification) observed during size exclusion chromatography (SEC). In some embodiments, SEC may be performed as described in Examples. In some embodiments, an anti-CD3 antibody or antigen-binding fragment as described herein may display % monomer in SEC of about 95% or higher, which indicates that the antibody is substantially existing as a monomer, i.e., not aggregating. In some embodiments, the % monomer may be about 97% or higher. In some embodiments, the % monomer may be about 98% or higher. In some embodiments, the % monomer may be about 99% or higher. In some embodiments, the % monomer may be about 99.5% or higher. Generally, the larger the % monomer value the more favorable the developability of the antibody.

In some embodiments, an anti-CD3 antibody or antigen-binding fragment as described herein may exhibit improved tolerance to an acidic environment or acidic stress, such as a pH of about 6 or below, about 5 or below, about 4 or below, or about 3.5 or below. In some embodiments, tolerance to low pH may be determined based on propensity to aggregate when exposed to low pH. In some embodiments, propensity to aggregate under a low pH may be determined based on % monomer values (e.g., monomer IgG or monomer Fab) observed during SEC after exposure to low pH. In some embodiments, such SEC for low pH tolerance test may be performed as described in Examples. In some embodiments, an anti-CD3 antibody or antigen-binding fragment as described herein may display % monomer in SEC of about 15% or higher after exposure to a low pH, which indicates that the antibody is substantially existing as a monomer, i.e., not aggregating. In some embodiments, the % monomer may be about 96% or higher. In some embodiments, the % monomer may be about 97% or higher. In some embodiments, the % monomer may be about 98% or higher. In some embodiments, the % monomer may be about 99% or higher. Generally, the larger the % monomer value after exposure to a low pH, the higher the tolerance to acidic stress, i.e., the more favorable the developability of the antibody. Without wishing to be bound by theory, improved tolerance to acidic stress may help provide longer shelf-life and/or improved in vivo stability (e.g., in an acidic cancer microenvironment).

In some embodiments, an anti-CD3 antibody or antigen-binding fragment as described herein may exhibit improved stability. In certain embodiments, stability may be assessed based on Tm values. In some cases, an anti-CD3 antibody or antigen-binding fragment as described herein may exhibit a Tm of about 65° C., or higher. In some embodiments, Tm may be determined using DSF, which may be performed as described in Examples, or any other appropriate methods. Generally, the higher the Tm is the more stable the antibody, i.e., mode favorable developability.

### Nucleic Acids, Vectors, and Cells

The present disclosure further encompasses nucleic acids (one nucleic acid or a combination of two (or more) nucleic acids) encoding the VH and VL of any of the anti-CD3 antibodies and antigen-binding fragments described herein. Exemplary nucleic acids include but are not limited to DNAs and RNAs.

In some embodiments, one nucleic acid molecule may encode both (i) an amino acid sequence comprising the VH and (ii) an amino acid sequence comprising the VL of the antibody. In certain embodiments, (i) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid molecule.

In some embodiments, the antibody-encoding nucleic acids may comprise: (i) a first nucleic acid encoding an amino acid sequence comprising the VH; and (ii) a second nucleic acid encoding an amino acid sequence comprising the VL.

In particular embodiments, the nucleic acid may comprise a VH-encoding sequence of SEQ ID NO: 650 and a VL-encoding sequence of SEQ ID NO: 660. In particular embodiments, the nucleic acid may comprise a VH-encoding sequence of SEQ ID NO: 550 and a VL-encoding sequence of SEQ ID NO: 560. In some cases, the nucleic acid may comprise a RNA which may comprise a RNA version of such sequences.

The present disclosure further encompasses vectors (one vector or a combination of two (or more) vectors) (e.g., expression vectors) encoding the VH and VL of any of the ani-CD3 antibodies and antigen-binding fragments described herein.

In some embodiments, one vector may comprise a nucleic acid which encodes both (i) an amino acid sequence comprising the VH and (ii) an amino acid sequence comprising the VL of the antibody. In certain embodiments, ii) and (ii) may be encoded on the same strand of the nucleic acid molecule. In some cases, (i) and (ii) may be encoded under a single promoter. In certain cases, (i) and (ii) may be encoded in the same direction (in some instances, (i) and (ii) may be transcribed into a single transcript, and in some instances (i) and (ii) may be transcribed into two separate transcripts). In certain cases, (i) and (ii) may be encoded in the opposite directions. In some cases, (i) and (ii) may be encoded under separate promoters. In certain embodiments, (i) and (ii) may be encoded on different strands within a nucleic acid.

In some embodiments, a vector may comprise: (i) a first vector comprising a nucleic acid encoding an amino acid sequence comprising the VH; and (ii) a second vector comprising a nucleic acid encoding an amino acid sequence comprising the VL.

It particular embodiments, a vector may comprise a nucleic acid comprising a VH-encoding sequence of SEQ ID NO: 650 and a nucleic acid comprising a VL-encoding sequence of SEQ ID NO: 660. In particular embodiments, a vector may comprise a nucleic acid comprising a VH-encoding sequence of SEQ ID NO: 550 and a nucleic acid comprising a VL-encoding sequence of SEQ ID NO: 560.

The present disclosure further provides an isolated, recombinant, and/or host cell comprising any of the anti-CD3 antibodies and antigen-binding fragments described above, comprising any of the nucleic acids described above, and/or comprising, transfected with, transduced with, or transformed with any of the vectors described above.

In some embodiments, a cell comprises, has been transfected with, has been transduced with, and/or has been transformed with: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

In some embodiments, the cell may be for manufacturing an anti-CD3 antibody or antigen-binding fragment according to the present disclosure. In some embodiments, the cells may be for administering to a subject.

In some embodiments, the cell is eukaryotic. In certain embodiments, the cell is mammalian, e.g., a Chinese Hamster Ovary (CHO) cell, human embryonic kidney (HEK) cell such as HEK293 cell, or lymphoid cell (e.g., Y0, NS0, Sp20 cell). In certain embodiments, the cell is yeast.

### Production of Anti-CD3 Antibodies and Cells Therefore

Any of the anti-CD3 antibodies and/or antigen-binding fragments described herein may be produced or manufactured using any appropriate methods, including recombinant methods, e.g., in vitro, ex vivo, or in vivo.

In some embodiments, the method of manufacturing an isolated, recombinant, and/or host cell comprising a nucleic acid encoding any of the anti-CD3 antibodies and antigen-binding fragments described herein or a population of such cells may comprise introducing the nucleic acids and/or the vector described herein into one or more cells.

Physical methods for introducing a nucleic acid into a cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning, A Laboratory Manual, Cold Spring Harbor Laboratory, New York). A preferred method for the introduction of a polynucleotide into a host cell is calcium phosphate transfection.

Biological methods for introducing a nucleic acid of interest into a cell include the use of DNA and RNA vectors. In certain embodiments, nucleic acid encoding the antibody may be isolated and inserted into one or more vectors (e.g., viral vectors, plasmids, and the like) for further cloning and/or expression in an isolated, recombinant, and/or cell. Such nucleic acids may be readily isolated and sequenced using conventional procedures (e.g., by using oligonucleotide probes that are capable of binding specifically to genes encoding the heavy and light chains of the antibody). Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a nucleic acid into a cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In some cases, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids, for example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In some embodiments, the method of manufacturing an anti-CD3 antibody or antigen-binding fragment according to the present disclosure may comprise culturing an isolated, recombinant, and/or host cell comprising a nucleic acid encoding the antibody, as provided above, under conditions suitable for expression of the antibody, and optionally harvesting, recovering, and/or purifying the antibody or antigen-binding fragment from the cell (or host cell culture medium).

Suitable host cells for cloning and/or expression of antibody-encoding vectors include prokaryotic or eukaryotic cells. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also, Chariton, Methods in Molecular Biology, Vol. 248 (B. K. C. Lo. ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See, e.g., Gerngross, Nat. Biotech. 22:1409-1414 (2004), and Li et al., Nat Biotech. 24:210-215 (2006); WO 2009/036379, WO 2010/105256; and WO 2012/009568.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod 23:243-251 (1980)); monkey kidney cells (CV1); African green monkey kidney cells (VERO-76); human cervical carcinoma cells (HELA), canine kidney cells (MDCK, buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0 For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Cells, such as hybridomas or other recombinant cells, that are producing antibodies or antigen-bunding fragments of the present disclosure may be grown using standard methods, in suitable culture medium for this purpose (such as D-MEM or RPMI-1640), or in vivo as ascites. Antibodies or antigen-binding fragments expressed and/or secreted by the cells can be separated from the cells, culture medium, ascites fluid, or serum using conventional immunoglobulin purification procedures, such as, but not limited to, protein A-Sepharose, hydroxyapatite chromatography, gel electrophoresis, dialysis, or affinity chromatography (Ma H. et al., Methods. 2017 Mar. 1:116:23-33. doi: 10.1016:j.ymeth.2016.11.008. Epub 2016 Nov. 18; Shukla A. A. et al. Trends Biotechnol 2010 May; 28(5):253-61. doi: 10.1016/j.tibtech.2010.02.001. Epub 2010 Mar. 19; Arora S. et al., Methods. 2017 Mar. 1; 116:84-94. doi: 10.1016/j.ymeth.2016.12.010. Epub 2016 Dec. 22).

Methods for expressing, isolating, and evaluating multispecific and bispecific antibodies and antibody fragments are also known in the art (for example, see Brinkmann U et al., MAbs. 2017 February-March; 9(2): 182-212. Published online 2017 Jan. 10. doi: 10.1080/19420862.2016.1268307; Dimasi N. et al. Methods. 2018 Aug. 11. pii: 51046-2023(18)30149-X. doi: 10.1016/j.ymeth.2018.08.004).

### Pharmaceutical Conditions

The present disclosure further encompasses pharmaceutical compositions comprising: (A) (i) any of the anti-CD3 antibodies and antigen-binding fragments described herein, (ii) a nucleic acid encoding such an anti-CD3 antibody or antigen-binding fragment, (iii) a vector encoding such an anti-CD3 antibody or antigen-binding fragment, and/or (iv) an isolated or recombinant cell comprising (i), (ii), and/or (iii); and (B) a pharmaceutically acceptable canier and/or excipient.

Therefore, in some embodiments, an anti-CD3 antibody or antigen-binding fragment may be an active ingredient; in some embodiments, a nucleic acid (e.g., DNA or RNA such as mRNA) or vector encoding an anti-CD3 antibody or antigen-binding fragment may be an active ingredient; and in some embodiments, a cell comprising a nucleic acid or a vector encoding an anti-CD3 antibody or antigen-binding fragment may be an active ingredient. In certain embodiments, when a mRNA is an active ingredient, the mRNA may be formulated into lipid nanoparticles, which may facilitate administration and delivery to cells of a subject receiving the compositions.

In some embodiments, a pharmaceutical composition comprising an anti-CD3 antibody and/or antigen-binding fragment as described herein may be prepared, e.g., by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions, optionally prepared for modified (e.g., sustained) release. Exemplary lyophilized antibody compositions are described in U.S. Pat. No. 6,267,958. Aqueous antibody formulations include those described in U.S. Pat. No. 6,171,586 and WO2006/044908, the latter compositions including a histidine-acetate buffer.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as scrum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDT A; sugars such as sucrose, mannitol, trehalose or sorbitol, salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble P11-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, a pharmaceutical composition according to the present disclosure may be used for any of the in vivo methods described herein.

### Additional Agents

In some embodiments, the pharmaceutical composition may be used alone without an additional agent.

In some embodiments, the pharmaceutical composition may further comprise (an) additional agent(s) (e.g., therapeutic agent(s)) or may be used in combination with (co-administered with or administered separately from) (an) additional agent(s) (e.g., therapeutic agent(s)) in a therapeutic regimen. The additional agents that may be combined with or used along with an anti-CD3 antibody according to the present disclosure may preferably be those with complementary activities that do not adversely affect each other and present in amounts that are effective for the purpose intended.

In certain embodiments, the additional agent(s) may be or may comprise a chemotherapeutic agent, gene therapy agent, DNA therapy agent, viral therapy agent, RNA therapy agent, immunotherapy agent, nanotherapy agent, monoclonal antibody, or a combination of the foregoing. In certain embodiments, the additional therapeutic agent(s) may be or may comprise an adjuvant or neoadjuvant. In certain embodiments, the additional therapeutic agent(s) may be or may comprise a small molecule enzymatic inhibitor or anti-metastatic agent.

In certain embodiments, the additional therapeutic agent(s) may be or may comprise a side-effect limiting agents (e.g., agents intended to lessen the occurrence and/or severity of side effects of treatment, such as anti-nausea agents, including but not limited to a neurokinin-1 receptor antagonist (NKI RA), serotonin receptor antagonist (5-HT3 RA), dexamethasone, olanzapine, and palonosetron, etc.).

Non-limiting exemplary additional agents may include a chemotherapy agent, an antibody-drug conjugate (ADC), an immunotherapy agent, and/or a biological modifier.

In certain embodiments, chemotherapy agents may be selected from alkylating agents, antimetabolites, plant alkaloids, and anti-cancer antibiotics, further optionally one or more selected from cyclophosphamide, cisplatin, carboplatin, oxalaplatin, etoposide, irinotecan, lurbinectedin, paclitaxel, docetaxel, cabazitaxel, altretamine, capecitabine, gemcitabine, ifosfamide, melphalan, pemetrexed, topotecan, vinorelbine, mitoxantrone, ixabepilone, eribulin, estramustine, vinblastine, vincristine, 5-fluorouracil (5-FU), doxorubicin, epirubicin, dactinomycin, or a derivative thereof. In certain embodiments, chemotherapy agents may be selected from cyclophosphamide, doxorubicin, vincristine, and prednisolone (CHOP).

In certain embodiments, ADC may be selected from an anti-CD79b antibody drug conjugate (such as anti-CD79b-MC-vc-PAB-MMAE or the anti-CD79b antibody drug conjugate described in any one of U.S. Pat. No. 8,088,378 and/or US 2014/0030280, or polatuzumab vedotin), an anti-CD19 antibody drug conjugate, an ani-CD22 antibody drug conjugate, an anti-CD45 antibody drug conjugate, and an anti-CD32 drug conjugate. A biological modifier may be selected from a BCL-2 inhibitor (such as GDC-0194/ABT-199), lenalidomide (REVLIMID®), a PI3K-delta inhibitor (such as idelalisib (ZYDELIG®)), a PD-1 axis binding antagonist, an agonist, e.g., agonist antibody, directed against an activating co-stimulatory molecule, e.g., CD40, CD226, CD28, OX40 (e.g., AgonOX), GITR, CD137 (also known as TNFRSF9, 4-1 BB, or ILA), CD27 (e.g., CDX-127), HVEM, or CD127, an antagonist, e.g., antagonist antibody, directed against an inhibitory co-stimulatory molecule, e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipilimumab (also known as MDX-010, MDX-101, or YERVOY®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g., a dominant-negative TGF beta type II receptor.

In certain embodiments, chemotherapy agents may be or may comprise an immune checkpoint inhibitor and/or a growth factor or growth factor receptor inhibitor, optionally an inhibitor of PD-L1, PD-1, CTLA-4, VISTA, EGF, EGFR, VEGF, and/or VEGFR, or an antibody or antigen-binding fragment against PD-L1, PD-1, CTLA-4, VISTA, EGF, EGFR, VEGF, and/or VEGFR, or an antibody or antigen-binding fragment against a cancer antigen.

In certain embodiments, the additional agent(s) may be or may comprise a chemotherapeutic agent, cytotoxic agent, an anti-hormonal agent, growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (TARCEVA™), platelet derived growth factor inhibitor(e.g., GLEEVEC™ (Imatinib Mesylate)), a COX-2 inhibitor (e.g., celecoxib), interferon, cytokine, antibody other than the anti-CD3 antibody of the disclosure, such as an antibody that bind to one or more of the following targets ErbB2, ErbB3, ErbB4, PDGFR-beta, BlyS, APRIL, BCMA VEGF, or VEGF receptor(s), TRAIL/Apo2, PD-1, PD-L1, PD-L2, or another bioactive or organic chemical agent. In certain embodiments, the additional therapeutic agent(s) may be or may comprise a glucocorticoid, optionally dexamethasone.

### Kits

In another aspect of the disclosure, kits useful for the treatment, prevention and/or diagnosis of a disease, a disorder, or a condition described herein is provided. The kit may comprise (A) a container comprising (i) any of the anti-CD3 antibodies and antigen-binding fragments described herein, (ii) a nucleic acid encoding such an anti-CD3 antibody or antigen-binding fragment, (iii) a vector encoding such an anti-CD3 antibody or antigen-binding fragment, (iv) an isolated or recombinant cell comprising (i), (ii), and/or (iii); and/or (v) any of the pharmaceutical compositions described herein; and (B) a label or package insert on or associated with the container. In certain embodiments, the container may comprise (an) additional agent(s), optionally a cytotoxic and/or therapeutic agent or any of the additional agents described herein.

In certain embodiments, the kit may comprise: (A-1) a first container comprising (i) any of the anti-CD3 antibodies and antigen-binding fragments described herein, (ii) a nucleic acid encoding such an anti-CD3 antibody or antigen-binding fragment, (iii) a vector encoding such an anti-CD3 antibody or antigen-binding fragment, (iv) an isolated or recombinant cell comprising (i), (ii), and/or (iii); and/or (v) any of the pharmaceutical compositions described herein; (A-2) a second container comprising (an) additional agent(s), optionally a cytotoxic and/or therapeutic agent or any of the additional agents described herein; and (B) a label or package insert on or associated with the container.

In certain embodiments, the kit may further comprise a second (or third) container comprising a pharmaceutically acceptable buffer, such as bacteriostatic water for injection (BWFI), phosphate-buffered saline, Ringer's solution and dextrose solution. In certain embodiments, the kit may further include other materials desirable from a commercial and user standpoint, including other buffers, diluents, filters, needles, and syringes.

Suitable containers may include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). The label or package insert may indicate that the composition is used for treating the condition of choice.

In some embodiments, a kit according to the present disclosure may be used for any of the in vivo methods described herein.

In certain embodiments, a kit as disclosed herein may be used alone or in combination with (an) additional agent(s). In certain embodiments, a kit may be for treating any disease, disorder, or condition described herein, such as a cell proliferative disorder (e.g., cancer) or an autoimmune disorder (e.g., arthritis, rheumatoid arthritis, colitis, inflammatory bowel disease, autoimmune type I diabetes, etc.).

### In Vivo Methods and Uses

The present disclosure further encompasses in vivo methods.

In some aspects, methods of and uses for treating a subject in need of such treatment and methods of and uses for treating or preventing a disease, disorder, or a condition in a subject are provided. Such methods and uses may comprise administering to the subject an effective amount of (i) any of the anti-CD3 antibodies and antigen-binding fragments described herein, (ii) a nucleic acid encoding such an anti-CD3 antibody or antigen-binding fragment, (iii) a vector encoding such an anti-CD3 antibody or antigen-binding fragment, (iv) an isolated or recombinant cell comprising (i), (ii), and/or (iii); and/or (v) any of the pharmaceutical compositions.

In some aspects, methods of and uses for eliciting cytotoxicity to a cell expressing a target molecule of interest are provided. Such methods and uses may comprise administering to the subject an effective amount of (i) a multispecific antibody or antibody fragment comprising at least one anti-CD3 antibody or antigen-binding fragment described herein, (ii) a nucleic acid encoding such a multispecific antibody or antibody fragment, (iii) a vector encoding such a multispecific antibody or antibody fragment, (iv) an isolated or recombinant cell comprising (i), (ii), and/or (iii); and/or (v) a pharmaceutical composition comprising (i), (ii), (iii), and/or (iv).

In any of the aspects and embodiments of methods and uses above, in some embodiments, the anti-CD3 antibody or antigen-binding fragment may be used to enhance immune function in an individual having a cell proliferative disorder or an autoimmune disorder. Following administration, such antibody may enhance immune function in an individual having a cell proliferative disorder or an autoimmune disorder by activating effector cells (e.g., T cells, e.g., CD8+ and/or CD4+ T cells including Tregs), expanding (increasing) the effector cell population, reducing the population of target cells (e.g., a cell expressing a second biological molecule recognized by an anti-CD3 antibody of the disclosure, such as a bispecific antibody), and/or killing a target cell (e.g., target tumor cell).

In a further aspect, methods of and uses for detecting CD3 or CD3-expressing cells in a subject, for example in a disease site (e.g., tumor site) or a potential disease site in a subject are provided. Such methods and uses may comprise administering to the subject an effective amount of any of the anti-CD3 antibodies and antigen-binding fragments described herein. In some embodiments, such methods and uses may be for diagnosis and/or detection. In certain embodiments, the diagnosis and/or detection may comprise determining the stage, severity, or immune profile of a disease or potential disease. In some cases, the immune profile may comprise a number of CD3-expressing cells in a tumor site. Without wishing to be bound by theory, the higher the number of CD3-expressing cells in a tumor the more likely the tumor may be treated effectively by an anti-CD3 antibody or antigen-binding fragment according to the present disclosure or another immunotherapy.

In any of the aspects and embodiments herein including methods and uses, the subject may be a mammal and, in particular, a human.

In any of the aspects and embodiments herein including methods and uses, the subject may have or may have a risk of developing a disease, a disorder, or a condition.

In any of the aspects and embodiments herein including methods and uses, the disease, disorder, or condition may be any appropriate disease, disorder, or condition including but not limited to those described herein. In some embodiments, the disease, disorder, or condition may be a proliferative disorder, cancer, an oncological disorder, an immune-oncological disorder, a neurological disorder, a cognitive disorder, a neurodegenerative disorder; and/or an inflammatory and/or autoimmune disorder (e.g., rheumatoid arthritis, colitis, inflammatory bowel disease, autoimmune type 1 diabetes, etc.).

In certain embodiments, the disease, disorder, or condition may be cancer. Without wishing to be bound by theory, tumor cells typically have an extracellular pH of around about 6.3-6.5, and the anti-CD3 antibodies and antigen-binding fragments described herein promote binding and activity preferentially in and around the tumor microenvironment due to their preferential CD3 binding at low(er) pH values, e.g., around pH 6. In certain embodiments, use of the anti-CD antibodies and antigen-binding fragments thereof may result in selective and sustained cytotoxic activity at or around the tumor site, thereby (i) reducing the effective amount needed for an intended purpose (e.g., therapy) and/or iii) reducing or eliminating off-target effects.

In certain embodiments, a RNA such as a mRNA encoding an anti-C D3 antibody or antigen-binding fragment or a composition comprising such a RNA may be administered. In some cases, the RNA (e.g. mRNA) may be formulated into lipid nanoparticles to facilitate administration and delivery to cells of a subject receiving the RNA.

### Administration Route and Dosing

In any of the aspects and embodiments of methods described herein, an effective amount of such an anti-CD3 antibody or antigen-binding fragment (and optionally any additional agent) or a pharmaceutical composition comprising such may be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In certain embodiments, the administration is subcutaneous administration, which may exhibit a less toxic response in the subject compared to intravenous injection. Dosing can be by any suitable mute (e.g., injections, such as intravenous or subcutaneous injections) and may depend in part on whether the administration is brief or chronic. Various dosing schedules including but not limited to single or multiple administrations over various time-points, bolus administration, and pulse infusion are contemplated herein.

Antibodies of the disclosure would be formulated (e.g., as a pharmaceutical composition), dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not, but may optionally be, formulated (e.g., as a pharmaceutical composition) with one or more agents currently used to prevent or treat the disorder in question. The effective amount of such other agents depends on the amount of antibody present in the composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a disease, a disorder, or a condition, the appropriate dosage of an antibody of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. In certain embodiments, the antibody may be suitably administered to the patient at one time or over a series of treatments.

As a general proposition, an effective amount (e.g., therapeutically effective amount) of the anti-CD3 antibody or antigen-binding fragment administered to humans may be in the range of about 0.01 to about 100 mg/kg of patient body weight whether by one or more administrations. In some embodiments, an antibody or antigen-binding fragment may be administered at about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg daily, for example. In one embodiment, an anti-CD3 antibody described herein is administered to a human at a dose of about 100 mg, about 200 mg, about 300 ng, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, for example, every week or every three weeks (e.g., such that the patient receives from about two to about twenty, or, for example, about six doses of the anti-CD3 antibody). An initial higher loading dose, followed by one or more lower doses, may be administered. The progress of this therapy is easily monitored by conventional techniques and assays.

### Monotherapy and Combination Therapy

In any of the aspects and embodiments of methods and uses described herein, an effective amount of such an anti-CD3 antibody or antigen-binding fragment may be administered alone to the subject.

In some embodiments, an effective amount of such an anti-CD3 antibody or antigen-binding fragment may be administered in combination with at least one additional agent to the subject. The additional agent(s) (e.g., therapeutic agents and/or adjuvants) may be or may comprise a chemotherapeutic agent, gene therapy agent, DNA therapy agent, viral therapy agent, RNA therapy agent, nanotherapy agent, monoclonal antibody, immunotherapy agent, and/or any agent such as but not limited to the additional agent(s) specifically described herein. In certain embodiments, the additional agent(s) may be contained in a pharmaceutical composition together with an anti-CD3 antibody or antigen-binding fragment according to the present disclosure. In certain embodiments, the additional agent(s) may not be contained in the pharmaceutical composition an anti-CD3 antibody or antigen-binding fragment according to the present disclosure but may be administered together (e.g., simultaneously) with the anti-CD3 antibody or antigen-binding fragment. In certain embodiments, the additional agent(s) may be administered separately from (e.g., prior to or following the administration of) the anti-CD3 antibody or antigen-binding fragment. In some cases, administration of the anti-CD3 antibody or antigen-binding fragment and administration of the additional agent(s) may occur within about one month, or within about one, two or three weeks, or within about one, two, three, four, live, or six days, of each other.

In some embodiments, an effective amount of such an anti-CD3 antibody or antigen-binding fragment may be administered in combination with at least one additional therapy to the subject. The additional therapy(ies) may be or may comprise radiation therapy (e.g., gamma irradiation), surgery, bone marrow transplantation, chemotherapy, or any combination of the foregoing. In certain embodiments, the additional therapy(ies) may be administered together (e.g., simultaneously) with the anti-CD3 antibody or antigen-binding fragment. In certain embodiments, the additional therapy(ies) may be administered separately from (e.g., prior to or following the administration of) the anti-CD3 antibody or antigen-binding fragment. In some cases, administration of the anti-CD3 antibody or antigen-binding fragment and administration of the additional therapy(ies) may occur within about one month, or within about one, two or three weeks, or within about one, two, three, four, five, or six days, of each other.

Any anti-CD3 antibodies and antigen-binding fragments, including bispecific anti-CD3 antibodies and antigen-binding fragments of the disclosure that bind to CD3 and a second biological molecule (e.g., a cell surface antigen, such as a tumor antigen) may be used in combination therapies described herein, such as in combination with radiation therapy.

Examples are provided below to illustrate embodiments of the present disclosure. These examples are not meant to constrain the present invention to any particular application or theory of operation.

### EXAMPLES

### Example 1: Novel pH-Dependent Anti-CD3 Antibodies

A pH-dependent anti-CD3 antibody, ADI-48587 (also referred to as Antibody No. 2 herein, initially disclosed in PCT/US20201036657) was previously identified from a library of antibody sequences varied from a non-pH-dependent anti-CD3 antibody, ADI-26906 (also referred to as Antibody No. 1 herein, initially disclosed in PCT/US2018/031705).

To obtain improved, pH-dependent anti-CD3 antibodies, the variable region sequences of ADI-48587 and ADI-26906 were varied. Novel anti-CD3 antibodies obtained included ADI-74968, ADI-74967, ADI-74966, ADI-74965, ADI-79842, ADI-79843, ADI-79848, ADI-79844, ADI-79845, ADI-79846, and ADI-79847 (also referred to as Antibody Nos. 6, 5, 4, 3, 7, 8, 9, 10, 11, 12, and 13, respectively). The amino acid sequences of the VH, VL, CDRs, and FRs and nucleic acid sequences encoding the VH and VL are shown in Appendix (Tables A-P). The consensus sequences shared among such antibodies are also shown in Appendix (Table Q).

### Example 2: Monovalent Binding Affinity at Different pHs—IgG, SPR

Anti-CD3 antibodies (Antibody Nos. 1-13) were produced as human IgG1 antibodies in yeast or Chinese hamster ovary (CHO) cells, and Fab fragments were generated from the IgG1 antibodies by papain digestion and purified IgGs produced in yeast were aglycosylated. CHO produced IgGs had standard CH2 glycosylation. Products were confirmed to exhibit expected mass by LC-MS analysis and >95% monomeric form by SEC-HPLC, with or without low pH stress (methods described below). Monovalent binding affinity to human or cynomolgus CD3εδ-Fc at pH 7.4 and pH 6.0 were measured via surface plasmon resonance (SPR) using the BIACORE® 8K (Cytiva, previously GE Healthcare Life Sciences) system and analyzed using BIACORE® Evaluation Software.

Results are shown in Table 1. As shown in the table, Fabs of Antibody Nos. 3-11 showed higher affinity (i.e., lower K_{D}) at pH 6.0 than at pH 7.4. Fabs of yeast-produced Antibody Nos. 3-6 bound to human CD3εδ-Fc at pH 6.0 but not at pH 7.4, with Nos. 3, 5, and 6 demonstrating higher affinity (i.e., lower K_{D}) than Ab No. 2 (the parent, pH-dependent antibody) at pH 6.0. Similar results were obtained with CHO-derived Fabs of Antibody Nos. 3-6, with increased affinity for human and cynomolgus CD3εδ-Fc at pH 6.0 when compared to affinity at pH 7.4 and increased affinity for human CD3εδ-Fc when compared with Ab No. 2.

TABLE 1
Binding affinity to CD3εδ-Fc analyzed by BIACORE ®

| | | Fab (produced in yeast) | | Fab (produced in CHO) | | Fab (produced in CHO) | |
|---|---|---|---|---|---|---|---|
| | | monovalent binding to | | monovalent binding to | | monovalent binding to | |
| | | human CD3εδ-Fc, | | human CD3εδ-Fc, | | cynomolgus CD3εδ-Fc, | |
| Antibody | ADI | K_{D} (M) | | K_{D} (M) | | K_{D} (M) | |
| No. | Name | pH 6.0 | pH 7.4 | pH 6.0 | pH 7.4 | pH 6.0 | pH 7.4 |
| 1 | ADI- | 1.90E−09 | 2.40E−09 | 1.25E−09 | 1.09E−09 | 1.90E−09 | 1.09E−09 |
| | 26906 | | | | | | |
| 2 | ADI- | 1.57E−08 | N.B. | 4.32E−08 | P.F. | 2.54E−08 | 6.27E−08 |
| | 48587 | | | | | | |
| 3 | ADI- | 1.05E−08 | N.B. | 1.09E−08 | P.F. | 1.13E−08 | P.F. |
| | 74965 | | | | | | |
| 4 | ADI- | 1.63E−08 | N.B. | 1.95E−08 | P.F. | 2.61E−08 | P.F. |
| | 74966 | | | | | | |
| 5 | ADI- | 8.45E−09 | N.B. | 8.16E−09 | 5.55E−08 | 1.16E−08 | 7.37E−08 |
| | 74967 | | | | | | |
| 6 | ADI- | 6.07E−09 | N.B. | 6.37E−09 | 4.75E−08 | 6.18E−09 | 4.65E−08 |
| | 74968 | | | | | | |
| 7 | ADI- | 6.16E−09 | 3.26E−08 | N.A. | N.A. | N.A. | N.A. |
| | 79842 | | | | | | |
| 8 | ADI- | 7.96E−10 | 5.90E−09 | N.A. | N.A. | N.A. | N.A. |
| | 79843 | | | | | | |
| 9 | ADI- | 5.76E−09 | 3.34E−08 | N.A. | N.A. | N.A. | N.A. |
| | 79848 | | | | | | |
| 10 | ADI- | 2.34E−09 | 3.45E−09 | N.A. | N.A. | N.A. | N.A. |
| | 79844 | | | | | | |
| 11 | ADI- | 1.96E−09 | 8.50E−09 | N.A. | N.A. | N.A. | N.A. |
| | 79845 | | | | | | |
| 12 | ADI- | P.F. | N.B. | N.A. | N.A. | N.A. | N.A. |
| | 79846 | | | | | | |
| 13 | ADI- | N.B. | N.B. | N.A. | N.A. | N.A. | N.A. |
| | 79847 | | | | | | |

P.F.: = Poor Fit. Binding was observed under the conditions of this assay but unable to be adequately fit to a 1:1 binding model.
N.B.: = No observable binding under the conditions of this assay; unable to assign a K_{D}.
N.A. = Not assessed.

### Example 3: Monovalent Binding Affinity at Different pHs—IgG1, BLI

Anti-CD3 antibodies Antibody Nos. 1-13 were produced as human IgG1 antibodies in yeast or CHO cells. IgGs produced in yeast were aglycosylated, CHO) produced IgGs had standard CH2 glycosylation. Binding affinity to human or cynomolgus CD3εδ-Fc at pH 7.4 and pH 6.0 was measured via biolayer interferometry (BLI) using the ForteBio OCTET® HTX system and analyzed using the ForteBio software.

Results are shown in Tables 2A and 2B. As shown in Table 2A, yeast-produced Antibody Nos. 3-12 showed higher affinity (i.e., lower K_{D}) to human CD3εδ-Fc at pH 6.0 than at pH 7.4, with higher affinity (i.e., lower K_{D}) to human CD3εδ-Fc than Ab No. 2 (the parent, pH-dependent antibody) at pH 6.0. Similar results were observed with CHO-produced Antibody Nos. 3-6. As shown in Table 2B, yeast-produced Antibody Nos. 3-10 and 12 bound to cynomolgus CD3εδ-Fc with higher affinity at pH 6.0 than pH 7.4. CHO-produced antibodies 3-6 bound to cynomolgus CD3εδ-Fc at pH 6.0 with higher affinity than Ab No. 2 and did not bind to cynomolgus CD3εδ-Fc at pH 7.4.

TABLE 2A
Binding affinity to human CD3εδ-Fc analyzed by OCTET ®

| | | yeast-produced IgG monovalent | | CHO-produced IgG monovalent | |
|---|---|---|---|---|---|
| | | binding to human CD3εδ-Fc; | | binding to human CD3εδ-Fc; | |
| | | IgG on AHC sensor, CD3εδ-Fc in | | CD3εδ-Fc on AHC sensor, Fab in | |
| | | solution (100 nM); | | solution (100 nM); | |
| Ab | | K_{D} (M) | | K_{D} (M) | |
| No. | ADI Name | pH 6.0 | pH 7.4 | pH 6.0 | pH 7.4 |
| 1 | ADI-26906 | 2.84E−09 | 4.35E−09 | 5.81E−09 | 6.03E−09 |
| 2 | ADI-48587 | 9.33E−09 | N.B. | 6.59E−08 | N.B. |
| 3 | ADI-74965 | 6.68E−09 | 1.37E−08 | 2.44E−08 | 1.42E−07 |
| 4 | ADI-74966 | 6.95E−09 | P.F. | 3.67E−08 | N.B. |
| 5 | ADI-74967 | 5.94E−09 | 1.4SE−08 | 2.49E−08 | P.F. |
| 6 | ADI-74968 | 5.23E−09 | 1.30E−08 | 1.48E−08 | 6.93E−08 |
| 7 | ADI-79842 | 6.52E−09 | 1.61E−08 | N.A. | N.A. |
| 8 | ADI-79843 | 1.60E−09 | 6.64E−09 | N.A. | N.A. |
| 9 | ADI-79848 | 6.47E−09 | 1.16E−08 | N.A. | N.A. |
| 10 | ADI-79844 | 3.60E−09 | 6.51E−09 | N.A. | N.A. |
| 11 | ADI-79845 | 1.04E−09 | 9.87E−09 | N.A. | N.A. |
| 12 | ADI-79846 | 8.09E−09 | P.F. | N.A. | N.A. |
| 13 | ADI-79847 | N.B. | N.B. | N.A. | N.A. |

Equilibrium dissociation constant (K_{D} = koff/kon); Data fit to a 1:1 binding model.
P.F. = Poor Fit. Binding was observed under the conditions of this assay but unable to be adequately fit to a 1:1 binding model.
N.B. = No observable binding under the conditions of this assay; unable to assign a K_{D}.
N.A. = Not assessed.

TABLE 2B
Binding affinity to cynomolgus CD3εδ-Fc and kinetics analyzed by OCTET ®

| | | yeast-produced IgG monovalent | | CHO-produced IgG monovalent | |
|---|---|---|---|---|---|
| | | binding to cynomolgus CD3εδ-Fc; | | binding to cynomolgus CD3εδ-Fc; | |
| | | IgG on AHC sensor, CD3εδ-Fc in | | IgG on AHC sensor, CD3εδ-Fc in | |
| | | solution (100 nM); | | solution (100 nM); | |
| Ab | | K_{D} (M) | | K_{D} (M) | |
| No. | ADI Name | pH 6.0 | pH 7.4 | pH 6.0 | pH 7.4 |
| 1 | ADI-26906 | 2.50E−09 | 4.10E−09 | 5.89E−09 | 5.87E−09 |
| 2 | ADI-48587 | N.B. | N.B. | 4.51E−07 | N.B. |
| 3 | ADI-74965 | 6.56E−09 | 2.18E−08 | 3.35E−08 | N.B. |
| 4 | ADI-74966 | 9.71E−09 | 4.76E−08 | 2.09E−07 | N.B. |
| 5 | ADI-74967 | 7.38E−09 | 2.19E−08 | 3.70E−08 | N.B. |
| 6 | ADI-74968 | 5.26E−09 | 2.05E−08 | 1.70E−08 | P.F. |
| 7 | ADI-79842 | 6.88E−09 | 1.79E−08 | N.A. | N.A. |
| 8 | ADI-79843 | 1.76E−09 | 5.66E−09 | N.A. | N.A. |
| 9 | ADI-79848 | 6.28E−09 | 1.45E−08 | N.A. | N.A. |
| 10 | ADI-79844 | 2.94E−09 | 5.42E−09 | N.A. | N.A. |
| 11 | ADI-79845 | N.B. | 8.06E−09 | N.A. | N.A. |
| 12 | ADI-79846 | 1.18E−08 | N.B. | N.A. | N.A. |
| 13 | ADI-79847 | N.B. | N.B. | N.A. | N.A. |

Equilibrium dissociation constant (K_{D} = koff/kon); Data fit to a 1:1 binding model.
N.B. = No observable binding under the conditions of this assay; unable to assign a K_{D}.
N.A. = Not assessed.

### Example 4: Cell Binding

Anti-CD3 antibodies Antibody Nos. 1-13 were produced as aglycosylated IgGs comprising a IgG1 Fc region in yeast cells. Binding to human or cynomolgus CD3-expressing cells (human CD3+ Jurkat cells or cynomolgus HSC-F cells, respectively) at pH 7.4 and pH 6.0 were measured by fluorescence-activated single cell sorting (FACS). Normalized cell binding (NCB) values were calculated based on median fluorescence intensity (MFI) values, CD3 negative Jurkat cells were used as a negative control.

Results are shown in Table 3. As shown Table 3, Antibody Nos. 3-12 showed higher binding (i.e., higher NCB values) to human CD3+Jurkat cells and cynomolgus HSC-F cells at pH 6.0 than at pH 7.4. Furthermore, compared to Ab No. 2 (the parent, pH-dependent antibody), Antibody Nos. 3 and 5-12 showed higher binding to human CD3+Jurkat cells at pH 6.0, Antibody Nos. 3 and 5-11 showed higher binding to and cynomolgus HSC-F cells at pH 6.0. None of the tested antibodies bound to the negative control cells.

TABLE 3
Cell binding measured by flow cytometry

| | | NCB at IgG = 100 nM | | | | | |
|---|---|---|---|---|---|---|---|
| Antibody | ADI | Human CD3 Jurkat | | Human CD3 (—) Jurkat | | Cyno HSC-F | |
| No. | Name | pH 6.0 | pH 7.4 | pH 6.0 | pH 7.4 | pH 6.0 | pH 7.4 |
| 1 | ADI-26906 | 203 | 149 | 4 | 4 | 560 | 167 |
| 2 | ADI-48587 | 149 | 0 | 1 | 0 | 437 | 0 |
| 3 | ADI-74965 | 181 | 39 | 3 | 0 | 551 | 74 |
| 4 | ADI-74966 | 131 | 0 | 2 | 0 | 378 | 0 |
| 5 | ADI-74967 | 185 | 34 | 3 | 0 | 512 | 81 |
| 6 | ADI-74968 | 197 | 62 | 3 | 1 | 548 | 106 |
| 7 | ADI-79842 | 208 | 139 | 3 | 2 | 575 | 172 |
| 8 | ADI-79843 | 207 | 149 | 5 | 3 | 603 | 174 |
| 9 | ADI-79848 | 200 | 136 | 3 | 1 | 550 | 173 |
| 10 | ADI-79844 | 196 | 147 | 4 | 3 | 603 | 160 |
| 11 | ADI-79845 | 200 | 149 | 4 | 3 | 581 | 167 |
| 12 | ADI-79846 | 65 | 4 | 1 | 0 | 235 | 2 |
| 13 | ADI-79847 | 0 | 0 | 0 | 0 | 2 | 0 |

NCB = (Sample MFI-Secondary Only MFD/Secondary Only MFI

### Example 5: Developability—PSR

Anti-CD3 antibody developability was assessed by polyspecificity analysis. Antibodies with high affinity for a target may otherwise fail in clinical settings where they also exhibit binding to multiple non-target entities. Antibody polyspecificity was assessed by measuring interaction with polyspecificity reagent (PSR). PSR was prepared as described in, e.g., WO 2014179363 and Xu et al., Protein Eng Des Sel, 26(10):663-670 (2013). In brief, 2.5 liters CHO-S cells were used as starting material. The cells were pelleted at 2,400×g for 5 min in 500 mL centrifuge bottles filled to 400 mL. Cell pellets were combined and then resuspended in 25 ml. Buffer B and pelleted at 2,400×g for 3 min. The buffer was decanted and the wash repeated one time. Cell pellets were resuspended in 3× the pellet volume of Buffer B containing 1×protease inhibitors (Roche, Complete, EDTA-tee) using a polytron homogenizer with the cells maintained on ice. The homogenate was then centrifuged at 2,400×g for 5 min and the supernatant retained and pelleted one additional time (2,400×g/5 min) to ensure the removal of unbroken cells, cell debris and nuclei; the resultant supernatant is the total protein preparation. The supernatant was then transferred into two Nalgene Oak Ridge 45 mL centrifuge tubes and pelleted at 40,000×g for 40 min at 4° C. The supernatants containing the Soluble Cytosolic Proteins (SCPs) were then transferred into clean Oak Ridge tubes, and centrifuged at 40,000×g one more time. In parallel, the pellets containing the membrane fraction (EMF) were retained and centrifuged at 40,000 for 20 min to remove residual supernatant. The EMF pellets were then rinsed with Buffer B. 8 mL Buffer B was then added to the membrane pellets to dislodge the pellets and transfer into a Dounce Homogenizer After the pellets were homogenized, they were transferred to a 50 mL conical tube and represented the final EMF preparation.

One billion mammalian cells (e.g. CHO, HEK293, Sf9) at ˜10⁶-10⁷ cells/mL were transferred from tissue culture environment into 4×250 mL conical tubes and pelleted at 550×g for 3 min. All subsequent steps were performed at 4° C. or on ice with ice-cold buffer. Cells were washed with 100 mL of PBSF (1×PBS+1 mg/mL BSA) and combined into one conical tube. After removing the supernatant, the cell pellet was then re-suspended in 30 mL Buffer B (50 mM HEPES, 0.15 M NaCl, 2 mM CaCl2, 5 mM KCl, 5 mM MgCl2, 10% Glycerol, pH 7.2) and pelleted at 550×g for 3 min. Buffer B supernatant was decanted and cells re-suspended in 3× pellet volume of Buffer B plus 2.5× protease inhibitor (Roche, cOmplete, EDTA-free). Protease inhibitors in Buffer B were included from here on forward. Cells were homogenized four times for 30 sec pulses (Polyton homogenizer, PT1200E) and the membrane fraction was pelleted at 40,060×g for 1 hour at 4° C. The pellet is rinsed with 1 mL. Buffer B; the supernatant is retained and represents the s. The pellet is transferred into a Dounce homogenizer with 3 mL of Buffer B and re-suspended by moving the pestle slowly up and down for 30-35 strokes. The enriched membrane fraction (EMF) is moved into a new collection tube, rinsing the pestle to collect all potential protein. Determine the protein concentration of the purified EMF using the Dc-protein assay kit (BioRad). To solubilize the EMF, transfer into. Solubilization Buffer (50 mM HEPES, 0.15 M NaCl, 2 mM CaCl2, 5 nM KCl, 5 mM MgCl2, 1% n-Dodecyl-b-D-Maltopyranoside (DDM), 1× protease inhibitor, pH 7.2) to a final concentration of 1 mg/mL. Rotate the mixture overnight at 4° C. rotating followed by centrifugation in a 50 mL Oak Ridge tube (Fisher Scientific, 050529-ID) at 40,000×g for 1 hour. Collect the supernatant which represents the soluble membrane proteins (SMPs) and quantify the protein yield as described above.

For biotinylation, prepare the NHS-LC-Biotin stock solution according to manufacturer's protocol (Pierce, Thermo Fisher). In brief, 20 μl of biotin reagent is added for every 1 mg of EMF sample and incubated at 4° C. for 3 hours with gentle agitation. Adjust the volume to 25 mL with Butter B and transfer to an Oak Ridge centrifuge tube. Pellet the biotinylated EMF (b-EMF) at 40,000×g for 1 hour, and rinse two times with 3 mL of Buffer C (Buffer B minus the glycerol) without disturbing the pellet. Remove the residual solution. Re-suspended the pellet with a Dounce homogenizer in 3 mL of Buffer C as described previously. The re-suspended pellet now represents biotinylated EMF (b-EMF). Solubilized as described above to prepare b-SMPs.

PSR Binding Analyse. Assays were performed generally as described in, e.g., Xu et al. Protein Eng Des Sel, 26(10):663-670 (2013). To characterize the PSR profile of monoclonal antibodies presented on yeast, two million IgG-presenting yeast were transferred into a 96-well assay plate and pellet at 3000×g for 3 min to remove supernatant. Re-suspend the pellet in 53 μl of freshly prepared 1:10 dilution of stock biotinylated PSRs (b-PSRs) and incubate on ice for 20 minutes. Wash the cells twice with 200 μl of cold PBSF and pellet re-suspended in 50 μl of secondary labeling mix (Extravidin-R-PE, anti-human LC-FITC, and propidium iodide). Incubate the mix on ice for 20 minutes followed by two washes with 200 μl ice-cold PBSF. Re-suspend the cells in 100 μl of ice-cold PBSF and run the plate on a FACSCanto (BD Biosciences) using HTS sample injector. Flow cytometry data was analyzed for mean fluorescence intensity in the R-PE channel and normalized to proper controls (antibodies with established PSR scores) in order to assess non-specific binding. Numerous methods for presentation or display of antibodies or antibody fragments on the surface of yeast have been described previously, all of which are consistent with this protocol (Blaise et al., Gene, 342(2):211-8 (2004), Boder and Wittrup, Nat Biotechnol., 15(6) 553-7 (1997), Kuroda and Ueda, Biotechnol Lett., 33(1):1-9 (2011), Orcutt and Wittrup, Springer Protocols—Antibody Engineering, 1:207-233 (2010), Rakestraw et al., Protein Eng Des Sel., 24(6):525-30 (2011), Sazinsky et al., Proc Natl. Acad Sci USA., 105(51):20167-72 (2008), Tasumi et al., Proc Natl Acad Sci USA., 106(31):12891-6 (2009).

Parental antibody No. 1 had the highest PSR score, indicating higher levels of non-target-specific binding. Antibody Nos. 2-6 demonstrated PSR scores≤0.1, a “clean” PSR score, indicating very low levels of non-target-specific binding.

### Example 6: Developability—HIC, AC-SINS, DLS, and Fab Tm

Anti-CD3 antibody developability was further determined by assessments of hydrophobicity, self-interaction, and stability. For these assessments, Antibody Nos. 1-6 were produced as human IgG1 antibodies in CHO cells. Fab fragments were generated from the IgG1 antibodies by papain digestion and purified.

### Hydrophobicity by HIC

Hydrophobicity of an antibody is one cause of antibody aggregation. To assess antibody hydrophobicity, IgG1 production samples were subjected to hydrophobic interaction chromatography (HIC) analyses. Briefly, IgG1 samples were buffer exchanged into 1 M ammonium sulfate and 0.1 M sodium phosphate at pH 6.5 using a Zeba 40 kDa 0.5 ml, spin column (Thermo Pierce, cat #87766). A salt gradient was established on a Dionex ProPac HTC-10 column from 1.8 M ammonium sulfate, 0.1 M sodium phosphate at pH 6.5 to the same condition without ammonium sulfate. The gradient ran for 17 min at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

HIC retention times for all of the tested IgG1s were 10.5 min, indicating clean to low hydrophobicity, i.e., a highly desirable developability profile.

### Self-Interaction by AC-SINS

Self-interaction was be measured in vitro by affinity-capture self-interaction nanoparticle spectroscopy (AC-SINS) using a previously described protocol (Liu y et al., MAbs. March-April 2014; 6(2):483-92). Briefly, polyclonal goat anti-human IgG Fc antibodies (capture; Jackson ImmunoResearch Laboratories) and polyclonal goat non-specific antibodies (non-capture; Jackson ImmunoResearch Laboratories) were buffer exchanged into 20 mM sodium acetate (pH 4.3) and concentrated to 0.4 mg/ml. A 4:1 volume ratio of capture:non-capture may be prepared and further incubated at a 1 (volume ratio with 20 nm gold nanoparticles (AuNP; Ted Pella Inc.) for 1 hour at room temperature. Thiolated PEG (Sigma-Aldrich) was then be used to block empty sites on the AuNP and filtered via a 0.22 μm PVDF membrane (Millipore). Coated particles were subsequently added to the test IgG1 antibody solution and incubated for 2 hours at room temperature before measuring absorbance from 510 to 570 nm on a plate reader. Data points were fit with a second-order polynomial in Excel to obtain wavelengths at maximum absorbance. Values were reported as the difference between plasmon wavelengths of the sample and background (Δλmax). Self-interaction levels were determined based on Δλmax. Self-interaction may be considered: low when Δλmax<5.0 nm; medium when Δλmax≥5.0 nm and 20.0 nm; and high when Δλmax≥20.0 nm.

Antibody Nos 2-6 had Δλmax of <5.0 indicating low self-interaction.

### Self-Interaction by DLS.

Self-interaction was also measured by dynamic light scattering (DLS). Diffusion Interaction Parameter (kD) of monoclonal antibodies, measured at concentrations lower than 12 mg/mL, has strong correlation with their solution behavior in very high concentrations (>100 mg/mL). Positive kD values indicate repulsive interaction among the molecules and has positive correlation with low viscosity at high concentration, in the same formulation buffer. kD values were obtained by measuring mutual diffusion coefficient for a series of different concentrations, by DLS. Specifically, DLS kD measurements at multiple concentrations between 0.5-12 mg/mL, in 10 mM Histidine buffer, pH 6.0 were taken. kD values 20 mL/g were considered as being associated with high viscosity or high opalescence.

Antibody Nos. 1-6 yielded kD values≥20 mL/g indicating low self-interaction.

### Fab Tm by DSF

Melting temperature (Tₘ) for Fabs of Antibody Nos. 1-6 was measured by differential scanning fluorometry (DSF) using a CFX96 Real-Time System from Bio-Rad. Briefly, 20 μL of 1 mg/mL sample was mixed with 10 μL of 20×SYPRO orange. The plate was scanned from 40° C., to 95° C., at a rate of 0.5° C./2 mm in a C1000 thermocycler (BioRad) to collect fret signal. The Fab Tm was assigned using the first derivative of the raw data from the Bio-Rad analysis software.

All Fabs tested had Tm values higher than 5° C., indicating high stability and thus desirable developability.

### Materials and Methods

The following Materials and Methods were employed in the Examples, unless otherwise noted.

Antibody yeast production and purification. Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect or CaptureSelect IgG-CH1 (GE Healthcare LifeSciences).

Antibody production in CHO cells and purification. Antibodies were produced as IgG1 by sub-cloning antibodies into a new expression vector followed by transfection and expression in CHO cells. Fab fragments were generated by papain digestion and purified over KappaSelect or CaptureSelect IgG-CH1 (GE Healthcare LifeSciences). The VH and VL encoding gene fragments (Integrated DNA Technologies) were subcloned into heavy- and light-chain pcDNA 3.4+ vectors (ThermoFisher). The corresponding vectors were transiently co-transfected into CHO-K1 suspension cells using standard methods well known in the art. In general, CHO-K1 cells grown to about 4×10*6 cells/mL were pelleted and resuspended in transfection medium. DNA plasmids (1.5 ug total DNA/mL) were incubated with PEIpro (1:2 final, PolyPlus, Cat #115-100) in transfection medium at room temperature before addition to the CHO-K1 cell suspension. Transfected cultures were fed and maintained at 32′C, shaking, until supernatant was harvested (at day 9) for purification. The cell culture supernatant was harvested by centrifugation and passed over Protein A agarose (MabSelect SuRe; GE Healthcare Life Sciences). The bound antibodies were then washed with PBS and eluted with buffer (200 mM acetic acid/50 mM NaCl, pH 3.5) into ⅛ volume 2 M Hepes, pH 8.0. The final products were buffer-exchanged into 25 mM Hepes and 150 mM sodium chloride, pH 7.3. Fab was generated using an overnight papain digest, followed by a CH1-resin purification step.

Hu and Cy CD3εδ Fc heterodimer antigen production. Recombinant heterodimeric CD3 Fe fusion antigens were produced in HEK 293 cells by co-transfection of plasmids encoding Hu CD3ε Fc (ectodomain, ECD, residues 22-126) and CD3δ Fc-HIS (ECD residues 22-100) or Cy CD3ε Fe (ECD residues 22-117) and CD3δ Fc-HIS (ECD residues 22-100) utilizing a heterologous signal peptide sequence (see Appendix Table U for sequences). Chromatographic separations were performed on a computer controlled ÅKTA Avant 150 preparative chromatography system (GE Healthcare Life Sciences) equipped with an integrated conductivity sensor, enabling in-line salt concentration monitoring during the run. Clarified culture supernatants were purified by Ni Sepharose 6 Fast Flow (GE Healthcare Life Sciences) which removes the CD3εε Fc homodimer. CD3εδ Fc-HIS heterodimer was resolved from CD366 Fc-HIS homodimer by Mono Q 10/100 GL, by a linear Tris-buffered KCl gradient at pH 8.5.

BIACORE® KD measurements (surface plasmon resonance; SPR). BIACORE® affinity measurements were performed generally as previously described. Briefly, human CD3εδ-Fc heterodimer produced as described above was immobilized to a NiNTA sensor chip in a BIACORE® 8K (Cytiva, previously GE Healthcare Life Sciences) to a response level of ˜500 RUs. Fabs were then injected at increasing concentrations, ranging from 18.75-300 nM, 1.56-25 nM, 0.25-100 nM, or 1.25-20 nM. The sensor chip was doubly regenerated between cycles using 0.35 M EDTA and 0.1 M NaOH. The resulting data were double reference subtracted and fit to a 1:1 binding model using BIACORE® Evaluation Software.

ForteBio K_{D} measurements (Biolayer interferometry; BLD. ForteBio affinity measurements were performed generally as previously described (Estep, P., et al., High throughput solution-based measurement of antibody-antigen affinity and epitope binning. MAbs, 2013. 5(2): p. 270-8.). IgGs were loaded online onto AHC sensors. Sensors were equilibrated off-line in assay buffer for 30 min and then monitored on-line for 60 seconds for baseline establishment. Sensors with loaded IgGs were exposed to 100 nM antigen (i.e., CD3) for 5 min, afterwards they were transferred to assay buffer for 5 min for off-rate measurement. Kinetics were analyzed using the 1:1 binding model.

ForteBio Kinetics FortBio OCTET® HTX instruments were used in 12 channel mode (8 sensors per channel, 96 sensors per experiment) with either AHC, SA, or AHQ sensors. Instrumentation was driven by manufacturer supplied software (versions 8.2 and 9.0). Sample names and concentrations were input into the plate data page, and sensor associated proteins were identified in the “information” column on the sensor data page. Kinetic experiments were collected with either a 90 or 180 s baseline, 180 s association phase, and 180 s dissociation phase. All files were saved into a shared network drive with a naming convention that identifies the format of the experiment.

Cell line propagation and cell labeling assays. Human Jurkat CD3+ cells (ATCC TIB-152) and Jurkat (CD3− cells (ATCC TIB-153) were obtained from ATCC. Cyno HSC-F cells were obtained from the NIH Non-human Primate Reagent Resource. All cell lines were cultured in RPMI 1640 GlutaMax media supplemented with 10% fetal bovine serum (FBS).

Cell binding assay, CD3+ and CD3− human Jurkat cells (ATCC) and cynomolgus HSC-F cells (NIH) were thawed and washed with cold PBSF buffer, pH 7.4 (PBS+0.1% BSA, pH 7.4). About 200,000 cells were aliquoted per well of a 96-well plate (FACS Assay Plate VWR BD 353263) and pelleted by centrifugation (5 minutes at 500×g). The cells were washed with either PBSF pH 7.4 or PBSF pH 6.0 (PBS+0.1% BSA, pH 6.0), and then resuspended in 100 μl in either PBSF pH 7.4 or PBSF pH 6.0 with IgG antibody (100 nM) produced in yeast as described above. The mixture (cells+antibody) was incubated for 20 minutes on ice, then washed twice with either PBSF pH 7.4 or PBSF pH 6.0. Cells were resuspended in 50 μl of propidium iodide (Roche; 1:500 dilution) and anti-human IgG-RPE (Southern Biotech; 1:100 dilution) prepared in either PBSF pH 7.4 or PBSF pH 6.0, then incubated for 20 minutes on ice in the dark before cells were washed twice with either PBSF pH 7.4 or PBSF pH 6.0. Binding was analyzed on FACS Canto 11.

PSR Preparation. Polyspecific reactivity reagent (PSR) was prepared as described in, e.g., WO 2014/179363 and Xu et al., Protein Eng Des Sel, 26(10):663-670 (2013). In brief, 2.5 liters CHO-S cells were used as starting material. The cells were pelleted at 2,400×g for 5 min in 500 mL centrifuge bottles filled to 400 mL Cell pellets were combined and then resuspended in 25 ml Buffer B and pelleted at 2,400×g for 3 min. The buffer was decanted and the wash repeated one time. Cell pellets were resuspended in 3× the pellet volume of Buffer B containing 1×protease inhibitors (Roche, Complete, EDTA-free) using a polytron homogenizer with the cells maintained on ice. The homogenate was then centrifuged at 2,400×g for 5 min and the supernatant retained and pelleted one additional time (2,400×g/6 min) to ensure the removal of unbroken cells, cell debris and nuclei; the resultant supernatant is the total protein preparation. The supernatant was then transferred into two Nalgene Oak Ridge 45 nil centrifuge tubes and pelleted at 40,000×g for 40 min at 4° C. The supernatants containing the Soluble Cytosolic Proteins (SCPs) were then transferred into clean Oak Ridge tubes, and centrifuged at 40,000×g one more time. In parallel, the pellets containing the membrane fraction (EMF) were retained and centrifuged at 40,006 for 20 min to remove residual supernatant. The EMF pellets were then rinsed with Buffer B. 8 mL Buffer B was then added to the membrane pellets to dislodge the pellets and transfer into a Dounce Homogenizer. After the pellets were homogenized, they were transferred to a 50 mL conical tube and represented the final EMF preparation.

One billion mammalian cells (e.g. CHO, HEK293, Sf9) at ˜10⁶-10⁷ cells/mL were transferred from tissue culture environment into 4×250 mL conical tubes and pelleted at 550×g for 3 min. All subsequent steps were performed at 4° C., or on ice with ice-cold buffers. Cells were washed with 100 mL of PBSF (1×PBS+1 mg/mL BSA) and combined into one conical tube. After removing the supernatant, the cell pellet was then re-suspended in 30 mL. Buffer B (50 mM HEPES, 0.15 M NaCl, 2 mM CaCl2, 5 mM KCl, 5 mM MgCl2, 10% Glycerol, pH 7.2) and pelleted at 550×g for 3 min. Buffer B supernatant was decanted and cells re-suspended in 3× pellet volume of Buffer B plus 2.5× protease inhibitor (Roche, cOmplete, EDTA-free). Protease inhibitors in Buffer B were included from here on forward. Cells were homogenized four times for 30 sec pulses (Polyton homogenizer, PT1200E) and the membrane fraction was pelleted at 40,000×g for 1 hour at 4° C. The pellet is rinsed with 1 mL Buffer B; the supernatant is retained and represents the s. The pellet is transferred into a Dounce homogenizer with 3 mL of Buffer B and re-suspended by moving the pestle slowly up and down for 30-35 strokes. The enriched membrane fraction (EMF) is moved into a new collection tube, rinsing the pestle to collect all potential protein. Determine the protein concentration of the purified EMF using the DC-protein assay kit (BioRad). To solubilize the EMF, transfer into. Solubilization Buffer (50 mM HEPES, 0.15 M NaCl, 2 mM CaCl2, 5 mM KCl, 5 mM MgCl2, 1% n-Dodecyl-b-D-Maltopyranoside (DUM), 1× protease inhibitor, pH 7.2) to a final concentration of 1 mg/mL. Rotate the mixture overnight at 4° C. rotating followed by centrifugation in a 50 mL Oak Ridge tube (Fisher Scientific, 050529-ID) at 40,000×g for 1 hour. Collect the supernatant which represents the soluble membrane proteins (SMPs) and quantify the protein yield as described above.

For biotinylation, prepare the NHS-LC-Biotin stock solution according to manufacturer's protocol (Pierce, Thermo Fisher). In brief, 20 μl of biotin reagent is added for every 1 mg of EMF sample and incubated at 4° C. for 3 hours with gentle agitation. Adjust the volume to 25 mL with Buffer B and transfer to an Oak Ridge centrifuge tube Pellet the biotinylated EMF (b-EMF) at 40,000×g for 1 hour, and rinse two times with 3 mL of Buffer C (Buffer B minus the glycerol) without disturbing the pellet. Remove the residual solution. Re-suspended the pellet with a Dounce homogenizer in 3 mL of Buffer C as described previously. The re-suspended pellet now represents biotinylated EMF (b-EMF). Solubilized as described above to prepare b-SMPs.

PSR. Binding Analyses. Assays were performed generally as described in, e.g., Xu et al. Protein Eng Des Sel, 26(10)-663-670 (2013). To characterize the PSR profile of monoclonal antibodies presented on yeast, two million IgG-presenting yeast were transferred into a 96-well assay plate and pellet at 3000×g for 3 min to remove supernatant. Re-suspend the pellet in 50 μl of freshly prepared 1:10 dilution of stock biotinylated PSRs (b-PSRs) and incubate on ice for 20 minutes. Wash the cells twice with 200 μl of cold PBSF and pellet re-suspended in 50 μl of secondary labeling mix (Extravidin-R PE, anti-human LC-FITC, and propidium iodide). Incubate the mix on ice for 20 minutes followed by two washes with 200 μl ice-cold PBSF. Re-suspend the cells in 100 μl of ice-cold PBSF and run the plate on a FACSCanto (BD Biosciences) using HTS sample injector. Flow cytometry data was analyzed for mean fluorescence intensity in the R-PE channel and normalized to proper controls (antibodies with established PSR scores) in order to assess non-specific binding. Numerous methods for presentation or display of antibodies or antibody fragments on the surface of yeast have been described previously, all of which are consistent with this protocol (Blaise et al., Gene, 342(2):211-8 (2004), Boder and Wittrup, Nat Biotechnol., 15(6):553-7 (1997), Kuroda and Ueda, Biotechnol Lett., 33(11:1-9 (2011), Orcutt and Wittrup, Springer Protocols: Antibody Engineering, 1:207-233 (2010). Rakestraw et al., Protein Eng Des Sci., 24(6):525-30 (2011), Sazinsky et al., Proc Natl Acad Sci USA., 105(51):20167-72 (2008), Tasumi et al., Proc Natl Acad Sci USA., 106(31):12891-6 (2009)). Polyspecificity was assessed based on the PSR score. Polyspecificity of antibodies was considered: clean (no polyspecificity) when 0.0≤PSR score<0.10; low when 0.10≤PSR score<0.33; medium when 0.33≤PSR score<0.66; and high when 0.66≤PSR score≤1.00.

HIC. IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 mm at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate. Hydrophobicity of antibodies was assessed based on the HIC retention time. Hydrophobicity was considered: clean to low when HIC retention time<10.5 minutes; medium when 10.5 minutes≤retention time<11.5 minutes; and high when 11.5 minutes≤retention time.

LC-MS. IgG1 samples were reduced by DTT, followed by middle down LCMS analysis on a Bruker maX is 4G mass spectrometer coupled with an Agilent 1100 HPLC (Agilent). A POROS R2 10 μm (2.1×30 mm) reversed phase column was used to remove salt in the samples. A fast LC flow at 2 mL/min allows the separation between sample and salt and elution of samples and regeneration of column to finish within a 2.1 min cycle A T-junction is used to deliver only 0.15 mL/min sample flow into the mass spectrometer for sample analysis. The Bruker maX is 4G mass spectrometer was run in positive ion mode with detection in the range of 750 to 2500 m/z. The remaining source parameters were set as follows; the capillary was set at 5500V, the Nebulizer at 4.0 Bar, dry gas at 4.0 l/min, and dry temp set at 200° C. The MS spectra were analyzed using Bruker Data Analysis version 4.1 and the deconvolution was accomplished using maximum entropy deconvolution with a mass range of 20 to 30 kDa. Tendency for heavy-heavy or heavy-light chain pairing failure was assessed based on LC-MS spectrogram. Presence of additional heavy chain and/or light chain peaks and/or a half-antibody peak indicates tendency of pairing failure. LC-MS was also used to confirm whether heavy and light chain mass matched expected mass based on amino acid sequences.

SEC. Agilent 1260 HPLC was employed to monitor the column chromatography (TSKgel Super SW mAb HTP column). The column was equilibrated with wash buffer (200 mM Sodium Phosphate. 250 mM Sodium Chloride pH 6.8) at a flow rate adjusted to 0.400 mL/min prior to use. Approximately 2-5 μg of an IgG1 or Fab protein sample was injected onto column. Protein migration was monitored at wavelength 280 nm. Total assay time was approximately 6 minutes. Data was analyzed using ChemStation software. Propensity of antibodies to aggregate was assessed based on % monomer in SEC chromatogram. Antibodies with % monomer of 95% or higher were considered to be substantially existing as a monomer, i.e., not aggregating.

Acidic stress tolerability, IgG1 samples were incubated at an acidic pH or a physiological pH and subjected to SEC-HPLC analyses. Briefly, IgG1 samples at 20 mg/mL were buffer exchanged into PBS (200 mM phosphate buffered with 250 mM sodium chloride, pH 7.0; or pH 3.5 buffer (50 mM sodium chloride, 200 mM acetic acid, pH 3.5). After 1 hour at room temperature (25° C.), buffer exchanged samples were diluted to 1 mg/mL in PBS (200 mM phosphate buffered with 250 mM sodium chloride, pH 7.0), and 2 μg of sample was injected into an Agilent 1260 Infinity analytical HPLC (Agilent, Santa Clara, CA) fitted with a TSKgel SuperSW mAb HTP column (TOSOH Bioscience, King of Prussia, PA, Product Code 22855). SEC data was collected and subjected to analysis using Agilent ChemStation software (Agilent, Santa Clara, CA). Tolerance of antibodies to acidic stress was evaluated based on propensity of antibodies to aggregate in an acidic condition assessed by % monomer in SEC chromatogram.

AC-SINS. Self-interaction was be measured in vitro by affinity-capture self-interaction nanoparticle spectroscopy (AC-SINS) using a previously described protocol (Liu y et al., MAbs. March-April 2014; 6(2):483-92). Briefly, polyclonal goat anti-human IgG Fc antibodies (capture; Jackson ImmunoResearch Laboratories) and polyclonal goat non-specific antibodies (non-capture; Jackson ImmunoResearch Laboratories) were buffer exchanged into 20 mM sodium acetate (pH 4.3) and concentrated to 0.4 mg/ml. A 4:1 volume ratio of capture:non-capture may be prepared and further incubated at a 1:9 volume ratio with 20 nm gold nanoparticles (AuNP, Ted Pella Inc.) for 1 hour at room temperature. Thiolated PEG (Sigma-Aldrich) was then be used to block empty sites on the AuNP and filtered via a 0.22 μm PVDF membrane (Millipore). Coated particles were subsequently added to the test IgG1 antibody solution and incubated for 2 hours at room temperature before measuring absorbance from 510 to 570 nm on a plate reader. Data points were fit with a second-order polynomial in Excel to obtain wavelengths at maximum absorbance. Values were reported as the difference between plasmon wavelengths of the sample and background (Δλmax). Self-interaction levels were determined based on Δλmax. Self-interaction was considered: low when Δλmax 5.0 nm; medium when Δλmax≥5.0 nm and <20.0 nm; and high when Δλmax≥20.0 nm.

DLS. Self-interaction was measured by dynamic light scattering (DLS). Diffusion Interaction Parameter (kD) of monoclonal antibodies, measured at concentrations lower than 12 mg/mL, has strong correlation with their solution behavior in very high concentrations (>100 mg/mL). Positive kD values indicate repulsive interaction among the molecules and has positive correlation with low viscosity at high concentration, in the same formulation buffer. kD values were obtained by measuring mutual diffusion coefficient for a series of different concentrations, by DLS. Specifically, DLS kD measurements at multiple concentrations between 0.5-12 mg/mL, in 10 mM Histidine buffer, pH 6.0 were taken. kD values<20 mL/g were considered as being associated with high viscosity or high opalescence.

DSF. Melting temperature (Tm) was measured by differential scanning fluorometry (DSF) using a CFX96 Real-Time System from Bio-Rad. Briefly, 20 μL of 1 mg/mL sample was mixed with 10 μL of 20×SYPRO orange. The plate was scanned from 40° C. to 95° C. at a rate of 0.5° C./2 min in a C1000 thermocycler (BioRad) to collect Fret signal. The Fab Tm was assigned using the first derivative of the raw data from the Bio-Rad analysis software. Antibodies with a Tm higher than 65° C. were considered to be stable.

An informal sequence listing is provided in Appendix, which provides the amino acid sequences of the VH, VL, CDRs, and FRs of the anti-CD3 antibodies analyzed in Examples and nucleic acid sequences encoding the VH and VL, amino acid sequences.

### APPENDIX

TABLE A
VH and VL amino acid sequences

| | | | | SEQ |
|---|---|---|---|---|
| Antibody | ADI | | | ID |
| No. | Name | Chain | Sequence | NO: |
| 1 | ADI-26906 | VH | 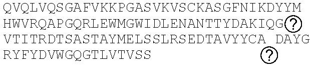 | 110 |
| | | VL | 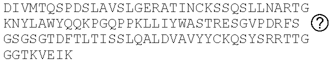 | 120 |
| 2 | ADI-48587 | VH | 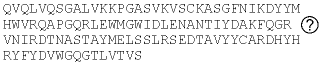 | 210 |
| | | VL | 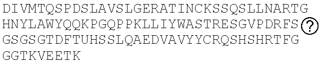 | 220 |
| 3 | ADI-74965 | | 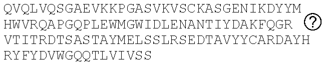 | 310 |
| | | VL | 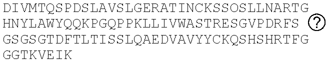 | 320 |
| 4 | ADI-74966 | VH | 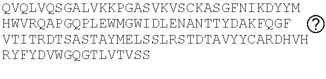 | 410 |
| | | VL | 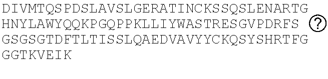 | 420 |
| 5 | ADI-74967 | VH | 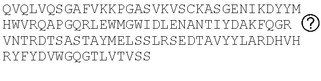 | 510 |
| | | VL | 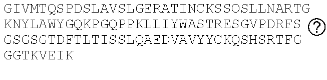 | 520 |
| 6 | ADI-74968 | VH | 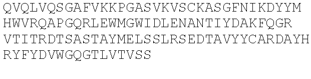 | 610 |
| | | VL | 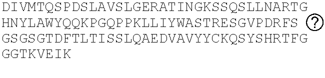 | 620 |
| 7 | ADI-79842 | 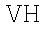 | 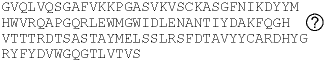 | 710 |
| | | VL | 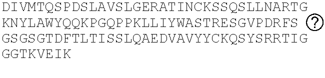 | 720 |
| 8 | ADI-79843 | VH | 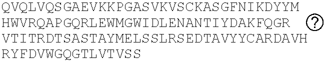 | 810 |
| | | VL | 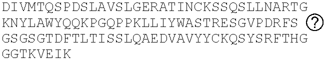 | 820 |
| 9 | ADI-79848 | VH | 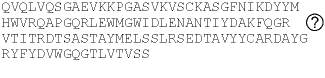 | 910 |
| | | VL | 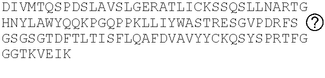 | 920 |
| 10 | ADI-79844 | VH | 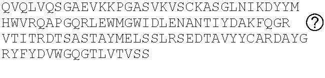 | 1010 |
| | | VL | 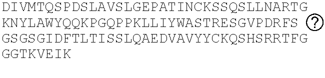 | 1020 |
| 11 | ADI-79845 | VH | 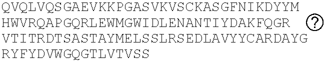 | 1110 |
| | | VL | 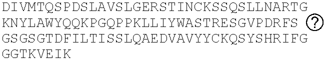 | 1120 |
| 12 | ADI-79846 | VH | 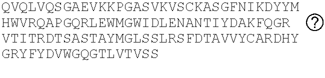 | 1210 |
| | | VL | 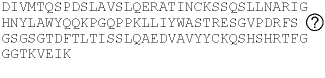 | 1220 |
| 13 | ADI-79847 | VH | 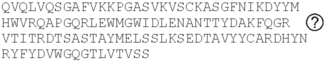 | 1310 |
| | | VL | 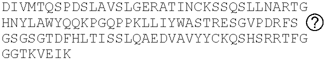 | 1320 |

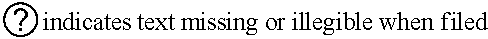

TABLE B
VH and VL nucleic acid sequences

| | | | | SEQ |
|---|---|---|---|---|
| Antibody | ADI | | | ID |
| No. | Name | Chain | Sequence | NO: |
| 1 | ADI-26906 | VH | 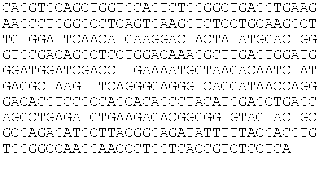 | 150 |
| | | VL | 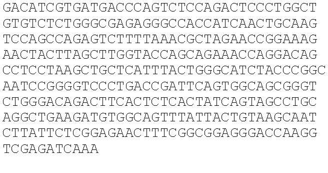 | |
| 2 | ADI-48587 | VH | 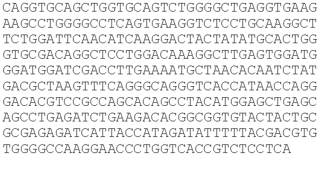 | 250 |
| | | VL | 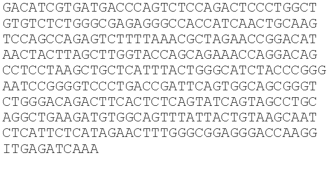 | 260 |
| 3 | ADI-74965 | VH | 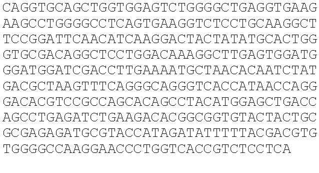 | 350 |
| | | VL | 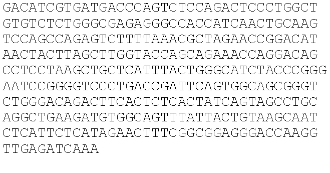 | 360 |
| 4 | ADI-74966 | VH | 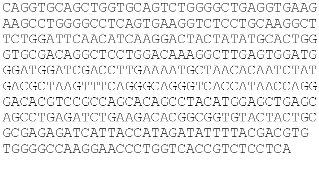 | 450 |
| | | VL | 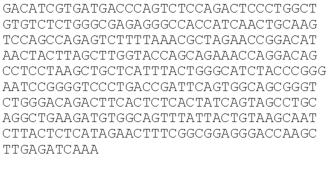 | 460 |
| 5 | ADI-74967 | VL | 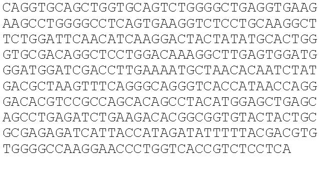 | 550 |
| | | VL | 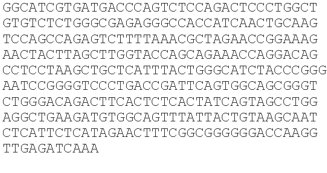 | 560 |
| 6 | ADI-74968 | VH | 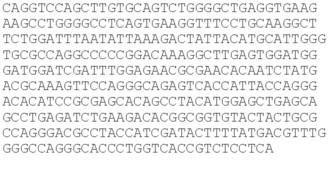 | 650 |
| | | VL | 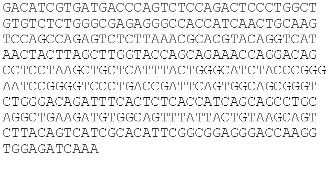 | 660 |
| 7 | ADI-79842 | VH | 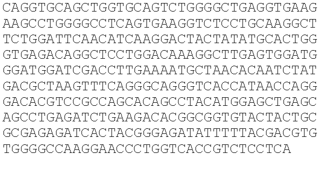 | 750 |
| | | VL | 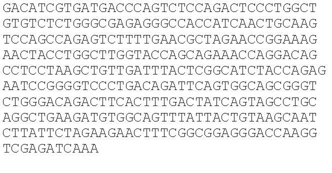 | 760 |
| 8 | ADI-79843 | VH | 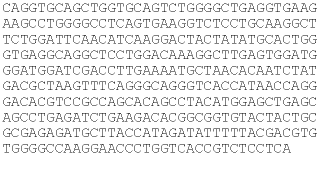 | 850 |
| | | VL | 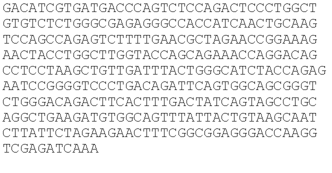 | 860 |
| 9 | ADI-79848 | VH | 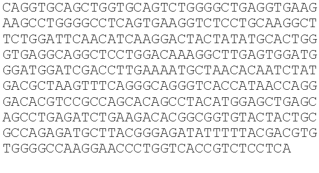 | 950 |
| | | VL | 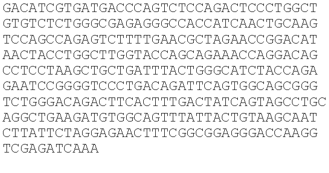 | 960 |
| 10 | ADI-79844 | VH | 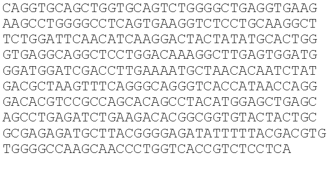 | 1050 |
| | | VL | 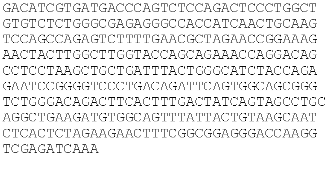 | 1060 |
| 11 | ADI-79845 | VH | 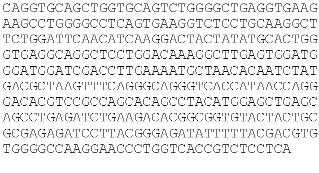 | 1150 |
| | | VL | 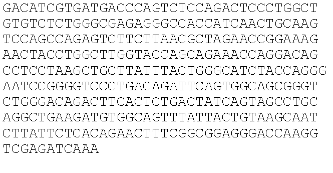 | 1160 |
| 12 | ADI-79846 | VH | 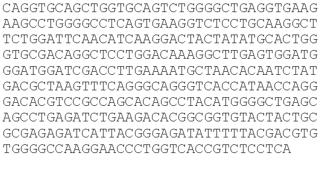 | 1250 |
| | | VL | 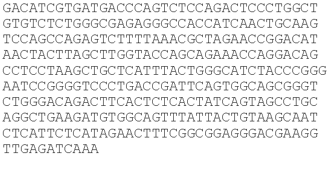 | 1260 |
| 13 | ADI-79847 | VH | 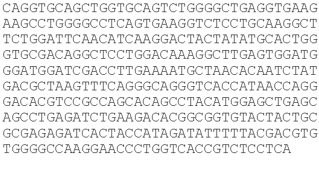 | 1350 |
| | | VL | 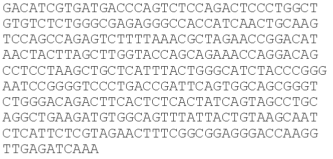 | 1360 |

TABLE C
FR-H1 amino acid sequences

| Antibody No. | ADI Name | Sequence | SEQ ID NO: |
|---|---|---|---|
| 1 | ADI-26906 | 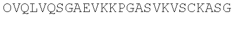 | 111 |
| 2 | ADI-48587 | 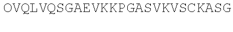 | 211 |
| 3 | ADI-74965 | 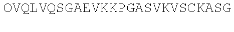 | 311 |
| 4 | ADI-74966 | 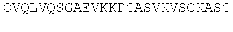 | 411 |
| 5 | ADI-74967 | 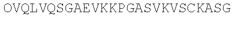 | 511 |
| 6 | ADI-74968 | 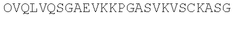 | 611 |
| 7 | ADI-79842 | 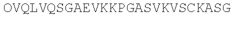 | 711 |
| 8 | ADI-79843 | 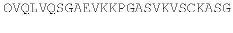 | 811 |
| 9 | ADI-79848 | 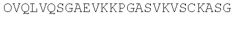 | 911 |
| 10 | ADI-79844 | 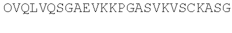 | 1011 |
| 11 | ADI-79845 | 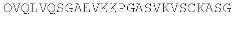 | 1111 |
| 12 | ADI-79846 | 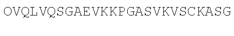 | 1211 |
| 13 | ADI-79847 | 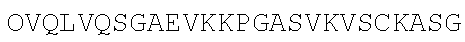 | 1311 |

TABLE D
CDR-H1 amino acid sequences

| Antibody | | | |
|---|---|---|---|
| No. | ADI Name | Sequence | SEQ ID NO: |
| 1 | ADI-26906 | 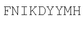 | 112 |
| 2 | ADI-48587 | 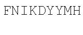 | 212 |
| 3 | ADI-74965 | 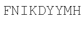 | 312 |
| 4 | ADI-74966 | 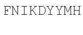 | 412 |
| 5 | ADI-74967 | 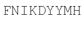 | 512 |
| 6 | ADI-74968 | 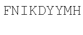 | 612 |
| 7 | ADI-79842 | 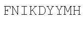 | 712 |
| 8 | ADI-79843 | 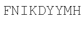 | 812 |
| 9 | ADI-79848 | 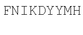 | 912 |
| 10 | ADI-79844 | 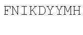 | 1012 |
| 11 | ADI-79845 | 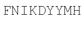 | 1112 |
| 12 | ADI-79846 | 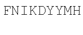 | 1212 |
| 13 | ADI-79847 | 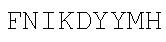 | 1312 |

Residues in bold indicate differences from the closest
germline (VH1-03)-encoded sequence.

TABLE E
FR-H2 amino acid sequences

| Antibody | | | |
|---|---|---|---|
| No. | ADI Name | Sequence | SEQ ID NO: |
| 1 | ADI-26906 | 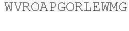 | 113 |
| 2 | ADI-48587 | 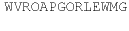 | 213 |
| 3 | ADI-74965 | 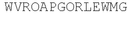 | 313 |
| 4 | ADI-74966 | 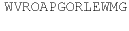 | 413 |
| 5 | ADI-74967 | 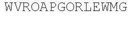 | 513 |
| 6 | ADI-74968 | 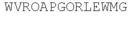 | 613 |
| 7 | ADI-79842 | 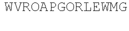 | 713 |
| 8 | ADI-79843 | 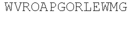 | 813 |
| 9 | ADI-79848 | 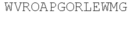 | 913 |
| 10 | ADI-79844 | 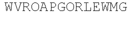 | 1013 |
| 11 | ADI-79845 | 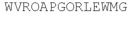 | 1113 |
| 12 | ADI-79846 | 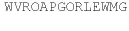 | 1213 |
| 13 | ADI-79847 | 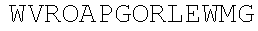 | 1313 |

TABLE F
CDR-H2 amino acid sequences

| Antibody | | | |
|---|---|---|---|
| No. | ADI Name | Sequence | SEQ ID NO: |
| 1 | ADI-26906 | 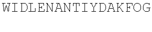 | 114 |
| 2 | ADI-48587 | 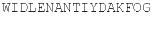 | 214 |
| 3 | ADI-74965 | 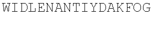 | 314 |
| 4 | ADI-74966 | 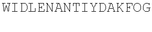 | 414 |
| 5 | ADI-74967 | 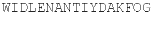 | 514 |
| 6 | ADI-74968 | 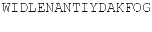 | 614 |
| 7 | ADI-79842 | 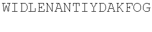 | 714 |
| 8 | ADI-79843 | 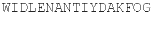 | 814 |
| 9 | ADI-79848 | 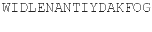 | 914 |
| 10 | ADI-79844 | 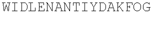 | 1014 |
| 11 | ADI-79845 | 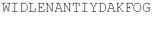 | 1114 |
| 12 | ADI-79846 | 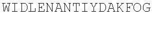 | 1214 |
| 13 | ADI-79847 | 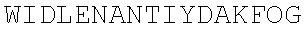 | 1314 |

Residues in bold indicate differences from the closest
germline (VH1-03)-encoded sequence.

TABLE G
FR-H3 amino acid sequences

| Antibody No. | ADI Name | Sequence | SEQ ID NO: |
|---|---|---|---|
| 1 | ADI-26906 | 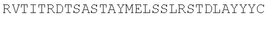 | 115 |
| 2 | ADI-48587 | 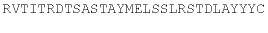 | 215 |
| 3 | ADI-74965 | 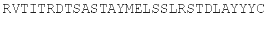 | 315 |
| 4 | ADI-74966 | 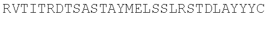 | 415 |
| 5 | ADI-74967 | 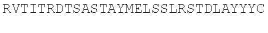 | 515 |
| 6 | ADI-74968 | 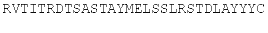 | 615 |
| 7 | ADI-79842 | 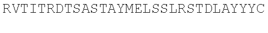 | 715 |
| 8 | ADI-79843 | 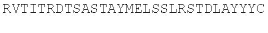 | 815 |
| 9 | ADI-79848 | 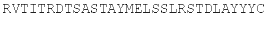 | 915 |
| 10 | ADI-79844 | 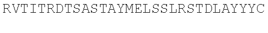 | 1015 |
| 11 | ADI-79845 | 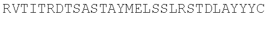 | 1115 |
| 12 | ADI-79846 | 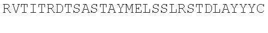 | 1215 |
| 13 | ADI-79847 | 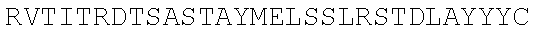 | 1315 |

Residues in bold indicate differences from the closest germline
(VH1-03)-encoded sequence.

TABLE H
CDR-H3 amino acid sequences

| Antibody No. | ADI Name | Sequence | SEQ ID NO: |
|---|---|---|---|
| 1 | ADI-26906 | 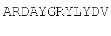 | 116 |
| 2 | ADI-48587 | 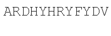 | 216 |
| 3 | ADI-74965 | 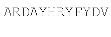 | 316 |
| 4 | ADI-74966 | 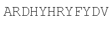 | 416 |
| 5 | ADI-74967 | 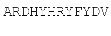 | 516 |
| 6 | ADI-74968 | 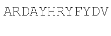 | 616 |
| 7 | ADI-79842 | 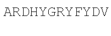 | 716 |
| 8 | ADI-79843 | 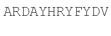 | 816 |
| 9 | ADI-79848 | 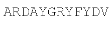 | 916 |
| 10 | ADI-79844 | 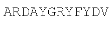 | 1016 |
| 11 | ADI-79845 | 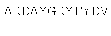 | 1116 |
| 12 | ADI-79846 | 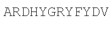 | 1216 |
| 13 | ADI-79847 | 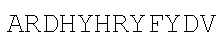 | 1316 |

Residues in italics indicate differences from the CDR-
H3 sequence of ADI-26906.

TABLE I
FR-H4 amino acid sequences

| Antibody No. | ADI Name | Sequence | SEQ ID NO: |
|---|---|---|---|
| 1 | ADI-26906 | 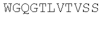 | 117 |
| 2 | ADI-48587 | 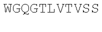 | 217 |
| 3 | ADI-74965 | 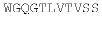 | 317 |
| 4 | ADI-74966 | 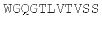 | 417 |
| 5 | ADI-74967 | 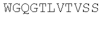 | 517 |
| 6 | ADI-74968 | 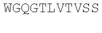 | 617 |
| 7 | ADI-79842 | 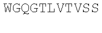 | 717 |
| 8 | ADI-79843 | 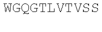 | 817 |
| 9 | ADI-79848 | 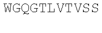 | 917 |
| 10 | ADI-79844 | 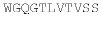 | 1017 |
| 11 | ADI-79845 | 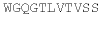 | 1117 |
| 12 | ADI-79846 | 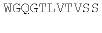 | 1217 |
| 13 | ADI-79847 | 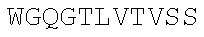 | 1317 |

TABLE J
FR-L1 amino acid sequences

| | | | SEQ |
|---|---|---|---|
| Antibody | | | ID |
| No. | ADI Name | Sequence | NO: |
| 1 | ADI-26906 | DIVMTQSPDSLAVSLGERATINC | 121 |
| 2 | ADI-48587 | DIVMTQSPDSLAVSLGERATINC | 221 |
| 3 | ADI-74965 | DIVMTQSPDSLAVSLGERATINC | 321 |
| 4 | ADI-74966 | DIVMTQSPDSLAVSLGERATINC | 421 |
| 5 | ADI-74967 | GIVMTQSPDSLAVSLGERATINC | 521 |
| 6 | ADI-74968 | DIVMTQSPDSLAVSLGERATINC | 621 |
| 7 | ADI-79842 | DIVMTQSPDSLAVSLGERATINC | 721 |
| 8 | ADI-79843 | DIVMTQSPDSLAVSLGERATINC | 821 |
| 9 | ADI-79848 | DIVMTQSPDSLAVSLGERATINC | 921 |
| 10 | ADI-79844 | DIVMTQSPDSLAVSLGERATINC | 1021 |
| 11 | ADI-79845 | DIVMTQSPDSLAVSLGERATINC | 1121 |
| 12 | ADI-79846 | DIVMTQSPDSLAVSLGERATINC | 1221 |
| 13 | ADI-79847 | DIVMTQSPDSLAVSLGERATINC | 1321 |

Residue in bold indicates a difference from the closest germiline (VK4-01)-encoded sequence.

TABLE K
CDR-L1 amino acid sequences

| | | | SEQ |
|---|---|---|---|
| Antibody | | | ID |
| No. | ADI Name | Sequence | NO: |
| 1 | ADI-26906 | KSSQSLLNARTGKNYLA | 122 |
| 2 | ADI-48587 | KSSQSLLNARTGHNYLA | 222 |
| 3 | ADI-74965 | KSSQSLLNARTGHNYLA | 322 |
| 4 | ADI-74966 | KSSQSLLNARTGHNYLA | 422 |
| 5 | ADI-74967 | KSSQSLLNARTGKNYLA | 522 |
| 6 | ADI-74968 | KSSQSLLNARTGHNYLA | 622 |
| 7 | ADI-79842 | KSSQSLLNARTGKNYLA | 722 |
| 8 | ADI-79843 | KSSQSLLNARTGKNYLA | 822 |
| 9 | ADI-79848 | KSSQSLLNARTGHNYLA | 922 |
| 10 | ADI-79844 | KSSQSLLNARTGKNYLA | 1022 |
| 11 | ADI-79845 | KSSQSLLNARTGKNYLA | 1122 |
| 12 | ADI-79846 | KSSQSLLNARTGHNYLA | 1222 |
| 13 | ADI-79847 | KSSQSLLNARTGHNYLA | 1322 |

Residues in bold indicate differences from the closest germiline (VK4-01) encoded sequence.
Residues in italics indicate differences from the CDR-L1 sequence of ADI-26906.

TABLE LFR-L2 amino acid sequences

| | | | SEQ |
|---|---|---|---|
| Antibody | | | ID |
| No. | ADI Name | Sequence | NO: |
| 1 | ADI-26906 | WYQQKPGQPPKLLIY | 123 |
| 2 | ADI-48587 | WYQQKPGQPPKLLIY | 223 |
| 3 | ADI-74965 | WYQQKPGQPPKLLIY | 323 |
| 4 | ADI-74966 | WYQQKPGQPPKLLIY | 423 |
| 5 | ADI-74967 | WYQQKPGQPPKLLIY | 523 |
| 6 | ADI-74968 | WYQQKPGQPPKLLIY | 623 |
| 7 | ADI-79842 | WYQQKPGQPPKLLIY | 723 |
| 8 | ADI-79843 | WYQQKPGQPPKLLIY | 823 |
| 9 | ADI-79848 | WYQQKPGQPPKLLIY | 923 |
| 10 | ADI-79844 | WYQQKPGQPPKLLIY | 1023 |
| 11 | ADI-79845 | WYQQKPGQPPKLLIY | 1123 |
| 12 | ADI-79846 | WYQQKPGQPPKLLIY | 1223 |
| 13 | ADI-79847 | WYQQKPGQPPKLLIY | 1323 |

TABLE M
CDR-L2 amino acid sequences

| Antibody | | | SEQ |
|---|---|---|---|
| No. | ADI Name | Sequence | ID NO: |
| 1 | ADI-26906 | WASTRES | 124 |
| 2 | ADI-48587 | WASTRES | 224 |
| 3 | ADI-74965 | WASTRES | 324 |
| 4 | ADI-74966 | WASTRES | 424 |
| 5 | ADI-74967 | WASTRES | 524 |
| 6 | ADI-74968 | WASTRES | 624 |
| 7 | ADI-79842 | WASTRES | 724 |
| 8 | ADI-79843 | WASTRES | 824 |
| 9 | ADI-79848 | WASTRES | 924 |
| 10 | ADI-79844 | WASTRES | 1024 |
| 11 | ADI-79845 | WASTRES | 1124 |
| 12 | ADI-79846 | WASTRES | 1224 |
| 13 | ADI-79847 | WASTRES | 1324 |

TABLE N
FR-13 amino acid sequences

| Antibody | | | SEQ |
|---|---|---|---|
| No. | ADI Name | Sequence | ID NO: |
| 1 | ADI-26906 | GVPDRFSGSGSGTDFTLTISSLQAEDVAVY | 125 |
| | | YC | |
| 2 | ADI-48587 | GVPDRFSGSGSGTDFTLTISSLQAEDVAVY | 225 |
| | | YC | |
| 3 | ADI-74965 | GVPDRFSGSGSGTDFTLTISSLQAEDVAVY | 325 |
| | | YC | |
| 4 | ADI-74966 | GVPDRFSGSGSGTDFTLTISSLQAEDVAVY | 425 |
| | | YC | |
| 5 | ADI-74967 | GVPDRFSGSGSGTDFTLTISSLQAEDVAVY | 525 |
| | | YC | |
| 6 | ADI-74968 | GVPDRFSGSGSGTDFTLTISSLQAEDVAVY | 625 |
| | | YC | |
| 7 | ADI-79842 | GVPDRFSGSGSGTDFTLTISSLQAEDVAVY | 725 |
| | | YC | |
| 8 | ADI-79843 | GVPDRFSGSGSGTDFTLTISSLQAEDVAVY | 825 |
| | | YC | |
| 9 | ADI-79848 | GVPDRFSGSGSGTDFTLTISSLQAEDVAVY | 925 |
| | | YC | |
| 10 | ADI-79844 | GVPDRFSGSGSGTDFTLTISSLQAEDVAVY | 1025 |
| | | YC | |
| 11 | ADI-79845 | GVPDRFSGSGSGTDFTLTISSLQAEDVAVY | 1125 |
| | | YC | |
| 12 | ADI-79846 | GVPDRFSGSGSGTDFTLTISSLQAEDVAVY | 1225 |
| | | YC | |
| 13 | ADI-79847 | GVPDRESGSGSGTDFTLTISSLQAEDVAVY | 1325 |
| | | YC | |

TABLE O
CDR-13 amino acid sequences

| Antibody | | | SEQ |
|---|---|---|---|
| No. | ADI Name | Sequence | ID NO: |
| 1 | ADI-26906 | KQSYSRRT | 126 |
| 2 | ADI-48587 | KQSHSHRT | 226 |
| 3 | ADI-74965 | KQSHSHRT | 326 |
| 4 | ADI-74966 | KQSYSHRT | 426 |
| 5 | ADI-74967 | KQSHSHRT | 526 |
| 6 | ADI-74968 | KQSYSHRT | 626 |
| 7 | ADI-79842 | KQSYSRRT | 726 |
| 8 | ADI-79843 | KQSYSRRT | 826 |
| 9 | ADI-79848 | KQSYSRRT | 926 |
| 10 | ADI-79844 | KQSHSRRT | 1026 |
| 11 | ADI-79845 | KQSYSHRT | 1126 |
| 12 | ADI-79846 | KQSHSHRT | 1226 |
| 13 | ADI-79847 | KQSHSRRT | 1326 |

Residues in italics indicate differences from the CDR-L3 sequence of ADI-26906.

TABLE P
FR-L4 amino acid sequences

| Antibody | | | SEQ |
|---|---|---|---|
| No. | ADI Name | Sequence | ID NO: |
| 1 | ADI-26906 | FGGGTKVEIK | 127 |
| 2 | ADI-48587 | FGGGTKVEIK | 227 |
| 3 | ADI-74965 | FGGGTKVEIK | 327 |
| 4 | ADI-74966 | FGGGTKVEIK | 427 |
| 5 | ADI-74967 | FGGGTKVEIK | 527 |
| 6 | ADI-74968 | FGGGTKVEIK | 627 |
| 7 | ADI-79842 | FGGGTKVEIK | 727 |
| 8 | ADI-79843 | FGGGTKVEIK | 827 |
| 9 | ADI-79848 | FGGGTKVEIK | 927 |
| 10 | ADI-79844 | FGGGTKVEIK | 1027 |
| 11 | ADI-79845 | FGGGTKVEIK | 1127 |
| 12 | ADI-79846 | FGGGTKVEIK | 1227 |
| 13 | ADI-79847 | FGGGTKVEIK | 1327 |

TABLE Q
Variable region amino acid sequences common among Antibody Indices Antibody
Nos. 1-13

| Variable | | | SEQ ID |
|---|---|---|---|
| region | Sequence | | NO: |
| FR-H1 | QVQLVQSGAEVKKPGASVKVSCKAS | | 11 |
| | G | | |
| CDR-H1 | FNIKDYYMH | | 12 |
| FR-H2 | WVRQAPGQRLEWMG | | 13 |
| CDR-H2 | WIDLENANTIYDAKFQG | | 14 |
| FR-H3 | RVTITRDTSASTAYMX₁LSSLRSEDTA | X₁ is E or G | 15 |
| | VYYC | | |
| CDR-H3 | ARDX₂YX₃RYFYDV | X₂ is A or H; and X₃ | 16 |
| | | is H or G | |
| FR-H4 | WGQGTLVTVSS | | 17 |
| FR-L1 | X₄VMTQSPDSLAVSLGERATINC | X₄ is D or G | 21 |
| CDR-L1 | KSSQSLLNARTGX₅NYLA | X₅ is H or K | 22 |
| FR-L2 | WYQQKPGQPPKLLIY | | 23 |
| CDR-L2 | WASTRES | | 24 |
| FR-L3 | GVPDRFSGSGSGTDFTLTISSLQAEDV | | 25 |
| | AVYYC | | |
| CDR-L3 | KQSX₆SX₇RT | X₆ is Y or H; and X₇ is | 26 |
| | | H or R | |
| FR-L4 | FGGGTKVEIK | | 27 |

TABLE R
SEQ ID NOS assigned to heavy chain variable sequences

| | | Amino acid | | | | | | | | Nucleic |
|---|---|---|---|---|---|---|---|---|---|---|
| Antibody | ADI | | FR- | CDR- | FR- | CDR- | FR- | CDR- | FR- | acid |
| No. | Name | VH | H1 | H1 | H2 | H2 | H3 | H3 | H4 | VH |
| 1 | ADI-26906 | 110 | 111 | 112 | 113 | 114 | 115 | 116 | 117 | 150 |
| 2 | ADI-48587 | 210 | 211 | 212 | 213 | 214 | 215 | 216 | 217 | 250 |
| 3 | ADI-74965 | 310 | 311 | 312 | 313 | 314 | 315 | 316 | 317 | 350 |
| 4 | ADI-74966 | 410 | 411 | 412 | 413 | 414 | 415 | 416 | 417 | 450 |
| 5 | ADI-74967 | 510 | 511 | 512 | 513 | 514 | 515 | 516 | 517 | 550 |
| 6 | ADI-74968 | 610 | 611 | 612 | 613 | 614 | 615 | 616 | 617 | 650 |
| 7 | ADI-79842 | 710 | 711 | 712 | 713 | 714 | 715 | 716 | 717 | 750 |
| 8 | ADI-79843 | 810 | 811 | 812 | 813 | 814 | 815 | 816 | 817 | 850 |
| 9 | ADI-79848 | 910 | 911 | 912 | 913 | 914 | 915 | 916 | 917 | 950 |
| 10 | ADI-79844 | 1010 | 1011 | 1012 | 1013 | 1014 | 1015 | 1016 | 1017 | 1050 |
| 11 | ADI-79845 | 1110 | 1111 | 1112 | 1113 | 1114 | 1115 | 1116 | 1117 | 1150 |
| 12 | ADI-79846 | 1210 | 1211 | 1212 | 1213 | 1214 | 1215 | 1216 | 1217 | 1250 |
| 13 | ADI-79847 | 1310 | 1311 | 1312 | 1313 | 1314 | 1315 | 1316 | 1317 | 1350 |

| | | | | | | | |
|---|---|---|---|---|---|---|---|
| Common sequence | 11 | 12 | 13 | 14 | 15 | 16 | 17 |

TABLE S
SEQ ID NOS assigned to light chain variable sequences

| | | Amino acid | | | | | | | | Nucleic |
|---|---|---|---|---|---|---|---|---|---|---|
| Antibody | ADI | | FR- | CDR- | FR- | CDR- | FR- | CDR- | FR- | acid |
| No. | Name | VL | L1 | L1 | L2 | L2 | L3 | L3 | L4 | VL |
| 1 | ADI-26906 | 120 | 121 | 122 | 123 | 124 | 125 | 126 | 127 | 160 |
| 2 | ADI-48587 | 220 | 221 | 222 | 223 | 224 | 225 | 226 | 227 | 260 |
| 3 | ADI-74965 | 320 | 321 | 322 | 323 | 324 | 325 | 326 | 327 | 360 |
| 4 | ADI-74966 | 420 | 421 | 422 | 423 | 424 | 425 | 426 | 427 | 460 |
| 5 | ADI-74967 | 520 | 521 | 522 | 523 | 524 | 525 | 526 | 527 | 560 |
| 6 | ADI-74968 | 620 | 621 | 622 | 623 | 624 | 625 | 626 | 627 | 660 |
| 7 | ADI-79842 | 720 | 721 | 722 | 723 | 724 | 725 | 726 | 727 | 760 |
| 8 | ADI-79843 | 820 | 821 | 822 | 823 | 824 | 825 | 826 | 827 | 860 |
| 9 | ADI-79848 | 920 | 921 | 922 | 923 | 924 | 925 | 926 | 927 | 960 |
| 10 | ADI-79844 | 1020 | 1021 | 1022 | 1023 | 1024 | 1025 | 1026 | 1027 | 1060 |
| 11 | ADI-79845 | 1120 | 1121 | 1122 | 1123 | 1124 | 1125 | 1126 | 1127 | 1160 |
| 12 | ADI-79846 | 1220 | 1221 | 1222 | 1223 | 1224 | 1225 | 1226 | 1227 | 1260 |
| 13 | ADI-79847 | 1320 | 1321 | 1322 | 1323 | 1324 | 1325 | 1326 | 1327 | 1360 |

| | | | | | | | |
|---|---|---|---|---|---|---|---|
| Common sequence | 21 | 22 | 23 | 24 | 25 | 26 | 27 |

TABLE T
Exemplary Constant Region amino acid sequences

| | | SEQ ID |
|---|---|---|
| | Sequences | NO: |
| Human IgG1 CH1, | ASTKGPSVFPLAPSSKSTSGGTAALGCLVKDYFPEPVTVSWNSGALTSG | 41 |
| reference 1 | VHTFPAVLQSSGLYSLSSVVTVPSSSLGTQTYICNVNHKPSNTKVDKKV | |
| Human IgG1 CH1, | ASTKGPSVFPLAPSSKSTSGGTAALGCLVKDYFPEPVTVSWNSGALTSG | 42 |
| reference 2 | VHTFPAVLQSSGLYSLSSVVTVPSSSLGTQTYICNVNHKPSNTKVDKRV | |
| Human IgG1 | EPKSCDKTHTCPPCP | 51 |
| hinge, reference | | |
| Human IgG1 CH2, | APELLGGPSVFLFPPKPKDTLMISRTPEVTCVVVDVSHEDPEVKFNWYV | 61 |
| reference | DGVEVHNAKTKPREEQYNSTYRVVSVLTVLHQDWLNGKEYKCKVSNK | |
| | ALPAPIEKTISKAK | |
| Human IgG1 CH3 | GQPREPQVYTLPPSRDELTKNQVSLTCLVKGFYPSDIAVEWESNGQPE | 71 |
| (356D and 358L) | NNYKTTPPVLDSDGSFFLYSKLTVDKSRWQQGNVFSCSVMHEALHNH | |
| | YTQKSLSLSPG | |
| Human IgG1 CH3 | GQPREPQVYTLPPSREEMTKNQVSLTCLVKGFYPSDIAVEWESNGQPE | 72 |
| (356E and 358M) | NNYKTTPPVLDSDGSFFLYSKLTVDKSRWQQGNVFSCSVMHEALHNH | |
| | YTQKSLSLSPG | |
| Human IgG1 CH3 | GQPREPQVYTLPPSRDELTKNQVSLTCLVKGFYPSDIAVEWESNGQPE | 73 |
| (356D, 358L, and | NNYKTTPPVLDSDGSFFLYSKLTVDKSRWQQGNIFSCSVMHEALHNHY | |
| V422I) | TQKSLSLSPG | |
| Human IgG1 CH3 | GQPREPQVYTLPPSRDELTKNQVSLTCLVKGFYPSDIAVEWESNGQPE | 74 |
| (356D, 358L, and | NNYKTTPPVLDSDGSFFLYSKLTVDKSRWQQGNVFSCSVMHEGLHNH | |
| A431G) | YTQKSLSLSPG | |
| Human IgG CK | RTVAAPSVFIFPPSDEQLKSGTASVVCLLNNFYPREAKVQWKVDNALQ | 81 |
| | SGNSQESVTEQDSKDSTYSLSSTLTLSKADYEKHKVYACEVTHQGLSSP | |
| | VTKSFNRGEC | |
| Human IgG CL | GQPKAAPSVTLFPPSSEELQANKATLVCLISDFYPGAVTVAWKADSSPV | 82 |
| | KAGVETTTPSKQSNNKYAASSYLSLTPEQWKSHRSYSCQVTHEGSTVE | |
| | KTVAPTECS | |

TABLE U
Human and Cynomolgus CD3ϵ Sequences

| | | SEQ ID |
|---|---|---|
| Name | Sequence | NO: |
| Hu CD3ϵ Fc | QDGNEEMGGITQTPYKVSISGTTVILTCPQYPGSEILWQHNDKNIGGDEDDK | 91 |
| | NIGSDEDHLSLKEFSELEQSGYYVCYPRGSKPEDANFYLYLRARVCENCMEMD | |
| | GGSDKTHTCPPCPAPELLGGPSVFLFPPKPKDTLMISRTPEVTCVVVDVSHED | |
| | PEVKFNWYVDGVEVHNAKTKPREEQYASTYRVVSVLTVLHQDWLNGKEYKC | |
| | KVSNKALPAPIEKTISKAKGQPREPQVYTLPPSRDELTKNQVSLTCLVKGFYPS | |
| | DIAVEWESNGQPENNYKTTPPVLDSDGSFFLYSKLTVDKSRWQQGNVFSCSV | |
| | MHEALHNHYTQKSLSLSPGK | |
| Cy CD3ϵ Fc | QDGNEEMGSITQTPYQVSISGTTVILTCSQHLGSEAQWQHNGKNKEDSGDR | 92 |
| | LFLPEFSEMEQSGYYVCYPRGSNPEDASHHLYLKARVCENCMEMDGGSDKT | |
| | HTCPPCPAPELLGGPSVFLFPPKPKDTLMISRTPEVTCVVVDVSHEDPEVKFN | |
| | WYVDGVEVHNAKTKPREEQYASTYRVVSVLTVLHQDWLNGKEYKCKVSNKA | |
| | LPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEAL | |
| | HNHYTQKSLSLSPGK | |

What is claimed is:

1. An anti-cluster of differentiation 3 (CD3) antibody or antigen-binding fragment thereof comprising:
(A) a heavy chain variable domain (VH) polypeptide comprising:
(a) a VH complementarity determining region 1 (CDR-H1) comprising the amino acid sequence of:
(i) the CDR-H1 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) FNIKDYYMH (SEQ ID NO: 12, 612, 512, 412, 312, 712, 812, 912, 1012, 1112, 1212, or 1312);
(b) a VH complementarity determining region 2 (CDR-H2) comprising the amino acid sequence of:
(i) the CDR-H2 contained in ADI-74968, ADI-74967, ADI-74966, ADI-74965, ADI-79842, ADI-79843, ADI-79848, ADI-79844, ADI-79845, ADI-79846, or ADI-70847; and/or
(ii) WIDLENANTIYDAKFQG (SEQ ID NO: 14, 614, 514, 414, 314, 714, 814, 414, 1014, 1114, 1214, or 1314); and/or
(c) a VH complementarity determining region 3 (CDR-H3) comprising the amino acid sequence of:
(i) the CDR-H3 contained in ADI-74968, ADI-74967, ADI-74966, ADI-7495, ADI-79842, ADI-79843, ADI-79848, ADI-79844, ADI-79845, ADI-79846, or ADI-79847;
(ii) ARDX₂Y X₃RYFYDV (SEQ ID NO: 16), wherein X₂ is A or H and X₂ is H or G; and/or
(iii) ARDAYHRYFYDV (SEQ ID NO: 616, 316, or 816), ARDHYHRYFYDV (SEQ ID NO: 516, 416, or 1316), ARDHYGRYFYDV (SEQ ID NO: 716 or 1216), or ARDAYGRYFYDV (SEQ ID NO: 916, 1016, or 1116); and/or
(B) a light chain variable domain (VL) polypeptide comprising:
(a) a VL complementarity determining region 1 (CDR-L1) comprising the amino acid sequence of:
(i) the CDR-L1 contained in ADI-74968, ADI-74967, ADI-74966, ADI-74965, ADI-79842, ADI-79843, ADI-79848, ADI-79844, ADI-79845, ADI-79846, or ADI-79847;
(ii) KSSQSLLNARTGX₃NYLA (SEQ ID NO: 22), wherein X₅ is H or K; and/or
(iii) KSSQSLLNARTGHNYLA (SEQ ID NO: 622, 422, 322, 922, 1222, or 1322) or KSSQSLLNARTGKNYLA (SEQ ID NO: 522, 722, 822, 1022, or 1122);
(b) a VL complementarity determining region 2 (CDR-L2) comprising the amino acid sequence of:
(i) the CDR-L2 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) WASTRES (SEQ ID NO: 24, 624, 524, 424, 324, 724, 824, 924, 1024, 1124, 1224, or 1324); and/or
(c) a VL complementarity determining region 3 (CDR-L3) comprising the amino acid sequence of:
(i) the CDR-L3 contained in ADI-74968, ADI-74967, ADI-74966, ADI-74965, ADI-79842, ADI-79843, ADI-79848, ADI-79844, ADI-79845, ADI-79846, or ADI-7847;
(ii) KQSX₆SX₇RT (SEQ ID NO: 26), wherein X₆ is Y or H and X₇ is H or R; and/or
(iii) KQSYSHRT (SEQ ID NO: 626, 426, or 1126), KQSHSHRT (SEQ ID NO: 526, 326, or 1226), KQSHSRRT (SEQ ID NO: 1026 or 1326), or KQSYSRRT (SEQ ID NO: 726, 826, or 926),
optionally wherein the anti-CD3 antibody and/or antigen-binding fragment:
(1) does not comprise:
(i) at least one of the CDR-H1, CDR-H2, CDR-H3, CDR-L1, CDR-L2, and CDR-L3 contained in ADI-48587; or
(ii) at least one of a CDR-H1 comprising FNIKDYYMH (SEQ ID NO: 12 or 212), a CDR-H2 comprising WIDLENANTIYDAKFQG (SEQ ID NO: 14 or 214), a CDR-H3 ARDHYHRYFYDV (SEQ ID NO: 216), a CDR-L1 comprising KSSQSLLNARTGHNYLA (SEQ ID NO: 222), a CDR-L2 comprising WASTRES (SEQ ID NO: 24 or 224), and a CDR-L3 comprising KQSHSHRT (SEQ ID NO: 2261; and
further optionally does not comprise:
(iii) at least one of the CDR-H3, CDR-L1, and CDR-L3 contained in ADI-48587; or
(iv) at least one of a CDR-H3 comprising ARDHYHRYFYDV (SEQ ID NO: 216), a CDR-L1 comprising KSSQSLLNARTGHNYLA (SEQ ID NO: 222), and a CDR-L3 comprising KQSHSHRT (SEQ ID NO: 226); and
(II) does not comprise:
(i) at least one of the CDR-H1, CDR-H2, CDR-H3, CDR-L1, CDR-L2, and CDR-L3 contained in ADI-26906; or
(ii) at least one of a CDR-H1 comprising FNIKDYYMH (SEQ ID NO: 12 or 112), a CDR-H2 comprising WIDLENANTIYDAKFQG (SEQ ID NO: 14 or 114), a CDR-H3 comprising ARDAYGRYFYDV (SEQ ID NO: 116), a CDR-L1 comprising KSSQSLLNARTGKNYLA (SEQ ID NO: 122), a CDR-L2 comprising WASTRES (SEQ ID NO: 24 or 124), and a CDR-L3 comprising KQSYSRRT (SEQ ID NO 126); and
further optionally does not comprise:
(iii) the CDR-H3, CDR-L1, and CDR-L3 contained in ADI-26906; or
(iv) a CDR-H3 comprising ARDAYGRYFYDV (SEQ ID NO: 116), a CDR-L1 comprising KSSQSLLNARTGKNYLA (SEQ ID NO: 122), and a CDR-L3 comprising KQSYSRRT (SEQ ID NO: 126).

2. The anti-CD3 antibody or antigen-binding fragment of claim 1, comprising:
(A) a VTH polypeptide comprising:
(a) a CDR-H1 comprising the amino acid sequence of:
(i) the CDR-H1 contained in ADI-74968, ADI-74967, ADI-74966, or ADI-74965; and/or
(ii) FNIKDYYMH (SEQ ID NO: 12, 612, 512, 412, or 312),
(b) a CDR-H2 comprising the amino acid sequence of:
(i) the CDR-H2 contained in ADI-74968, ADI-74967, ADI-74966, or ADI-74965; and/or
(ii) WIDLENANTIYDAKFQG (SEQ ID NO: 14, 614-514, 414, or 314); and/or
(c) a CDR-H3 comprising the amino acid sequence of:
(i) the CDR-H3 contained in ADI-74968, ADI-74967, ADI-74966, or ADI-74965,
(ii) ARDX₂Y X₃RYFYDV (SEQ ID NO: 16), wherein X₂ is A or H and X₃ is H; and/or
(iii) ARDAYHRYFYDV (SEQ ID NO: 616 or 316) or ARDHYHRYFYDV (SEQ ID NO: 516 or 416); and/or
(B) a VL polypeptide comprising:
(a) a CDR-L1 comprising the amino acid sequence of:
(i) the CDR-L1 contained in ADI-74968, ADI-74967, ADI-74966, or ADI-74965;
(ii) KSSQSLLNARTGX₅NYLA (SEQ ID NO: 22), wherein X₅ is H or K; and/or
(iii) KSSQSLLNARTGHNYLA (SEQ ID NO: 622, 422, or 322) or KSSQSLLNARTGKNYLA (SEQ ID NO: 522);
(b) a CDR-L2 comprising the amino acid sequence of:
(i) the CDR-L2 contained in ADI-74968, ADI-74907, ADI-74966, or ADI-74965; and/or
(ii) WASTRES (SEQ ID NO: 24, 624, 524, 424, or 324); and/or
(c) a CDR-L3 comprising the amino acid sequence of:
(i) the CDR-L3 contained in ADI-74968, ADI-74967, ADI-74966, or ADI-74965;
(ii) KQSX₆SX₇RT (SEQ ID NO: 26), wherein X₆ is Y or H and X₇ is H; and/or
(iii) KQSYSHRT (SEQ ID NO: 626 or 426) or KQSHSHRT (SEQ ID NO: 526 or 326).

3. The anti-CD3 antibody or antigen-binding fragment of any one of the foregoing claims, comprising:
(A) a VH polypeptide comprising:
(a) a CDR-H1 comprising the amino acid sequence of (i) the CDR-H1 contained in ADI-74968; and/or (ii) FNIKDYYMH (SEQ ID NO: 12 or 612);
(b) a CDR-H2 comprising the amino acid sequence of: (i) the CDR-H2 contained in ADI-74968; and/or (ii) WIDLEENANTIYDAKFQOG (SEQ ID NO: 14 or 614); and/or
(c) a CDR-H3 comprising the amino acid sequence of: (i) the CDR-H3 contained in ADI-74968; and/or (it) ARDAYHRYFYDV (SEQ ID NO: 616); and/or
(B) a VL polypeptide comprising:
(a) a CDR-L1 comprising the amino acid sequence of: (i) the CDR-L1 contained in ADI-74968; (ii) KSSQSLLNARTGHNYLA (SEQ ID NO: 622);
(b) a CDR-L2 comprising the amino acid sequence of: i) the CDR-L2 contained in ADI-74968; and/or (ii) WASTRES (SEQ ID NO: 24 or 624); and/or
(c) a CDR-L3 comprising the amino acid sequence of: (i) the CDR-L3 contained in ADI-74968; (ii) KQSYSHRT (SEQ ID NO: 626).

4. The anti-CD3 antibody or antigen-binding fragment of any one of the foregoing claims, comprising:
(A) a VH polypeptide comprising:
(a) a CDR-H1 comprising the amino acid sequence of: (i) the CDR-H1 contained in ADI-74967; and/or (ii) FNIKDYYMH (SEQ ID NO: 12 or 512);
(b) a CDR-H2 comprising the amino acid sequence of: (i) the CDR-H2 contained in ADI-74967; and/or (ii) WIDLENANTIYDAKFQG (SEQ ID NO: 14 or 514); and/or
(c) a CDR-H3 comprising the amino acid sequence of: (i) the CDR-H3 contained in ADI-74967; (ii) ARDHYHRYFYDV (SEQ ID NO: 516); and/or
(B) a VL polypeptide comprising:
(a) a CDR-L1 comprising the amino acid sequence of: (i) the CDR-L1 contained in ADI-74967; (ii) KSSQSLLNARTGKNYLA (SEQ ID NO: 522);
(b) a CDR-L2 comprising the amino acid sequence of: (i) the CDR-L2 contained in ADI-74967; and/or (ii) WASTRES (SEQ ID NO: 24, 624, 524); and/or
(c) a CDR-L3 comprising the amino acid sequence of: (i) the CDR-L3 contained in ADI-74967; (ii) KQSHSHRT (SEQ ID NO: 526).

5. The ani-CD3 antibody or antigen-binding fragment of any one of the foregoing claims, comprising:
(A) a VH polypeptide comprising said CDR-H1, said CDR-H2, and said CDR-H3; and/or
(B) a VL polypeptide comprising said CDR-L1, said CDR-L2, and said CDR-L3.

6. The anti-CD3 antibody or antigen-binding fragment of any one of the foregoing claims, comprising:
(A) a VH polypeptide comprising said CDR-H1, said CDR-H2, and said CDR-H3; and
(B) a VL polypeptide comprising said CDR-L1, said CDR-L2, and said CDR-L3.

7. The anti-CD3 antibody or antigen-binding fragment of any one of the foregoing claims, comprising:
(I) (i) the CDR-H1, CDR-H2, CDR-H3, CDR-L1, CDR-L2, and the CDR-L3 contained in ADI-74968; or
(ii) a CDR-H1 comprising FNIKDYYMH (SEQ ID NO: 12 or 612), a CDR-H2 comprising WIDLENANTIYDAKFQG (SEQ ID NO: 14 or 614), a CDR-H3 comprising ARDAYHRYFYDV (SEQ ID NO: 616), a CDR-L1 comprising KSSQSLLNARTGHNYLA (SEQ ID NO: 622), a CDR-L2 comprising WASTRES (SEQ ID NO: 24 or 624), and a CDR-L3 comprising KQSYSHRT (SEQ ID NO: 626);
(II) (i) the CDR-H1, CDR-H2, CDR-H3, CDR-L, CDR-L2, and the CDR-L3 contained in ADI-74967; or
(ii) a CDR-H1 comprising FNIKDYYMH (SEQ ID NO: 12 or 512), a CDR-H2 comprising WIDLENANTIYDAKFQG (SEQ ID NO: 14 or 514), a CDR-H3 comprising ARDHYHRYFYDV (SEQ ID NO: 516), a CDR-L1 comprising KSSQSLLNARTGKNYLA (SEQ ID NO: 522), a CDR-L2 comprising WASTRES (SEQ ID NO: 24 or 524), and a CDR-L3 comprising KQSHSHRT (SEQ ID NO: 526);
(III) (i) the CDR-H1, CDR-H2, CDR-H3, CDR-L1, CDR-L2, and the CDR-L3 contained in ADI-74966; or
(ii) a CDR-H1 comprising FNIKDYYMH (SEQ ID NO: 12 or 412), a CDR-H2 comprising WIDLENANTIYDAKFQG (SEQ ID NO: 14 or 414), a CDR-H3 comprising ARDHYHRYFYDV (SEQ ID NO: 416), a CDR-L1 comprising KSSQSLLNARTGKNYLA (SEQ ID NO: 422), a CDR-L2 comprising WASTRES (SEQ ID NO: 24 or 424), and a CDR-L3 comprising KQSYSHRT (SEQ ID NO: 426);
(IV) (i) the CDR-H1, CDR-H2, CDR-H3, CDR-L1, CDR-L2, and the CDR-L3 contained in ADI-74965; or
(ii) a CDR-H1 comprising FNIKDYYMH (SEQ ID NO: 12 or 312), a CDR-H2 comprising WIDLENANTIYDAKFQG (SEQ ID NO: 14 or 314), a CDR-H3 comprising ARDAYHRYFYDV (SEQ ID NO: 316), a CDR-L1 comprising KSSQSLLNARTGHNYLA (SEQ ID NO: 322), a CDR-L2 comprising WASTRES (SEQ ID NO: 24 or 324), and a CDR-L3 comprising KQSHSHRT (SEQ ID NO: 326);
(V) (i) the CDR-H1, (CDR-H2, CDR-H3, CDR-L1, CDR-L2, and the CDR-L3 contained in ADI-79842; or
(ii) a CDR-H1 comprising FNIKDYYMH (SEQ ID NO: 12 or 712), a CDR-H2 comprising WIDLENANTIYDAKFQG (SEQ ID NO: 14 or 714), a CDR-H3 comprising ARDHYGRYFYDV (SEQ ID NO: 716), a CDR-L1 comprising KSSQSLLNARTGKNYLA (SEQ ID NO: 722), a CDR-L2 comprising WASTRES (SEQ ID NO: 24 or 724), and a CDR-L3 comprising KQSYSRRT (SEQ ID NO: 726);
(VI) (i) the CDR-H1, CDR-H2, CDR-H3, CDR-L1, CDR-L2, and the CDR-L3 contained n ADI-79843; or
(ii) a CDR-H1 comprising FNIKDYYMH (SEQ ID NO: 12 or 812), a CDR-H2 comprising WIDLENANTIYDAKFQG (SEQ ID NO: 14 or 814), a CDR-H3 comprising ARDAYHRYFYDV (SEQ ID NO: 816), a CDR-L1 comprising KSSQSLLNARTGKNYLA (SEQ ID NO: 822), a CIDR-L2 comprising WASTRES (SEQ ID NO: 24 or 824), and a CDR-L3 comprising KQSYSRRT (SEQ ID NO: 826);
(VII) (i) the CDR-H1, CDR-H2, CDR-H3, CDR-L1, CDR-L2, and the CDR-L3 contained in ADI-79848; or
(ii) a CDR-H1 comprising FNIKDYYMH (SEQ ID NO: 12 or 912), a CDR-A12 comprising WIDLENANTIYDAKFQG (SEQ ID NO: 14 or 914), a CDR-H3 comprising ARDAYGRYFYDV (SEQ ID NO: 916), a CDR-L1 comprising KSSQSLLNARTGHNYLA (SEQ ID NO: 922), a CDR-L2 comprising WASTRES (SEQ ID NO: 24 or 924), and a CDR-L3 comprising KQSYSRRT (SEQ ID NO: 926);
(Vill) (i) the CDR-H1, CDR-H2, CDR-H3, CDR-L1, CDR-L2, and the CDR-L3 contained in ADI-79844; or
(ii) a CDR-H1 comprising FNIKDYYMH (SEQ ID NO: 12 or 1012), a CDR-H2 comprising WIDLENANTIYDAKFQG (SEQ ID NO: 14 or 1014), a CDR-H3 comprising ARDAYGRYFYDV (SEQ ID NO: 1016), a CDR-L1 comprising KSSQSLLNARTGKNYLA (SEQ ID NO: 1022), a CDR-L2 comprising WASTRES (SEQ ID NO: 24 or 1024), and a CDR-L3 comprising KQSYSRRT (SEQ ID NO 1026);
(IX) (i) the CDR-H1, CDR-H2, CDR-H3, CDR-L1, CDR-L2, and the CDR-L3 contained in ADI-79845; or
(ii) a CDR-H1 comprising FNIKDYYMH (SEQ ID NO: 12 or 1112), a CDR-H2 comprising WIDLENANTIYDAKFQG (SEQ ID NO: 14 or 1114), a CDR-H3 comprising ARDAYGRYFYDV (SEQ ID NO: 1116), a CDR-L1 comprising KSSQSLLNARTGKNYLA (SEQ ID NO: 1122), a CDR-L2 comprising WASTRES (SEQ ID NO: 24 or 1124), and a CDR-L3 comprising KQSYSHRT (SEQ ID NO: 1126);
(X) (i) the CDR-H1, CDR-H2, CDR-H3, CDR-L1, CDR-L2, and the CDR-L3 contained in ADI-79846; or
(ii) a CDR-H1 comprising FNIKDYYMH (SEQ ID NO: 12 or 1212), a CDR-H12 comprising WIDLEENANTIYDAKFQOG (SEQ ID NO: 14 or 1214), a CDR-H3 comprising ARDAYGRYFYDV (SEQ ID NO: 1216), a CDR-L1 comprising KSSQSLLNARIGHNYLA (SEQ ID NO: 1222), a CDR-L2 comprising WASTRES (SEQ ID NO: 24 or 1224), and a CDR-L3 comprising KQSHSHRT (SEQ ID NO: 1226);
(XI) (i) the CDR-H1, CDR-H12, CDR-H3, CDR-L1, CDR-L2, and the CDR-L3 contained in ADI-79846; or
(ii) a CDR-H1 comprising FNIKDYYMH (SEQ ID NO: 12 or 1312), a CDR-H2 comprising WIDLENANTIYDAKFQG) (SEQ ID NO: 14 or 1314), a CDR-H3 comprising ARDHYHRYFYDV (SEQ ID NO: 1316), a CDR-L1 comprising KSSQSLLNARTGHNYLA (SEQ ID NO: 1322), a CDR-L2 comprising WASTRES (SEQ ID NO: 24 or 1324), and a CDR-L3 comprising KQSHSRRT (SEQ ID NO: 1326).

8. The anti-CD3 antibody or antigen-binding fragment of any one of the foregoing claims, wherein:
(A) the V14 polypeptide comprises:
(a) a VH framework region 1 (FR-H1) comprising the amino acid sequence of:
(i) the FR-H1 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) QVQLVQSGAEVKKPGASVKVSCKASG (SEQ ID NO: 11, 611, 511, 411, 311, 711, 811, 911, 1011, 1111, 1211, or 1311);
(b) a VH framework region 2 (FR-H2) comprising the amino acid sequence of:
(i) the FR-H2 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) WVRQAPGQRLEWMG (SEQ ID NO: 13, 613, 513, 413, 313, 713, 813, 913, 1013, 1113, 1213, or 1313);
(c) a VH framework region 3 (FR-H3) comprising the amino acid sequence of:
(i) the FR-H3 contained in ADI-74968, ADI-74967, ADI-74966, ADI-74965, ADI-79842, ADI-79843, ADI-79848, ADI-79844, ADI-79845, ADI-79846, or ADI-79847;
(ii) RVTITRDTSASTAYMELSSLRSEDTAVYYC (SEQ ID NO: 15), wherein X₁ is E or G; and/or
(iii) RVTITRDTSASTAYMELSSLRSEDTAVYYC (SEQ ID NO: 615, 515, 415, 315, 715, 815, 915, 1015, 1115, or 1315) or RVTITRDTSASTAYMGLSSLRSEDTAVYYC (SEQ ID NO: 1215); and/or
(d) a VHT framework region 4 (FR-H4) comprising the amino acid sequence of:
(i) the FR-H4 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) WGQGTLVTVSS (SEQ ID NO: 17, 617, 517, 417, 317, 717, 817, 917, 1017, 1117, 1217, or 1317), and/or
(B) the VL polypeptide comprises:
(a) a VL framework region 1 (FR-L1) comprising the amino acid sequence of:
(i) the FR-L1 contained in ADI-74968, ADI-74967, ADI-74966, ADI-74965, ADI-79842, ADI-79843, ADI-79848, ADI-79844, ADI-79845, ADI-79846, or ADI-79847;
(ii) X₄IVMTQSPDSLAVSLGERATINC (SEQ ID NO: 21), wherein X₄ is D or G; and/or
(iii) DIVMTQSPDSLAVSLGERATINC (SEQ ID NO: 621, 421, 321, 721, 821, 921, 1021, 1121, 1221, or 1321) or GIVMTQSPDSLAVSLGERATINC (SEQ ID NO: 521);
(b) a VL framework region 2 (FR-L2) comprising the amino acid sequence of:
(i) the FR-L2 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) WYQQKPGQPPKLLIY (SEQ ID NO: 23, 623, 523, 423, 323, 723, 823, 923, 1023, 1123, 1223, or 1323);
(c) a VL framework region 3 (FR-L3) comprising the amino acid sequence of:
(i) the FR-L3 contained in ADI-74968, ADI-74967, ADI-74966, ADI-74965, ADI-79842, ADI-79843, ADI-79848, ADI-79844, ADI-79845, ADI-79846, or ADI-79847; and/or
(ii) GVPDRFSGSGSGTDFTLTISSLQAEDVAVYYC (SEQ ID NO: 25, 625, 525, 425, 325, 725, 825, 925, 1025, 1125, 1225, or 1325); and/or
(d) a VL framework region 4 FR-L4) comprising the amino acid sequence of:
(i) the FR-L4 contained in ADI-74968, ADI-74967, ADI-74966, ADI-74965, ADM-79842, ADI-79843, ADI-79848, ADI-79844, ADI-79845, ADI-79846, or ADI-79847; and/or
(ii) FGGGTKVEIK (SEQ ID NO: 27, 627, 527, 427, 327, 727, 827, 927, 1027, 1127, 1227, or 1327),
or the anti-CD3 antibody or antigen-binding fragment comprising a VH and/or a VL that comprises any combination of the foregoing VH and VL framework regions.

9. The anti-CD3 antibody or antigen-binding fragment of any one of the foregoing claims, comprising:
(I) (i) the FR-H1, FR-H2, FR-H3, FR-H4, FR-L1, FR-L2, FR-L3, and the FR-L4 contained in ADI-74968, ADI-74966, ADI-74965, ADI-79842, ADI-79843, ADI-79848, ADI-79844, ADI-79845, or ADI-79847; or
(ii) a FR-H1 comprising QVQLVQSGAEVKKPGASVKVSCKASG (SEQ ID NO: 11, 611, 411, 311, 711, 811, 911, 1011, 1111, or 1311), a FR-H2 comprising WVRQAPGQRLEWMG (SEQ ID NO: 13, 613, 413, 313, 713, 813, 913, 1013, 1113, or 1313), a FR-H3 comprising RVTITRDTSASTAYMELSSLRSEDTAVYYC (SEQ ID NO: 615, 415, 315, 715, 815, 915, 1015, 1115, or 1315), a FR-H4 comprising WGQGTLVTVSS (SEQ ID NO: 17, 617, 417, 317, 717, 817, 917, 1017, 1117, or 1317), a FR-L1 comprising DIVMTQSPDSLAVSLGERATINC (SEQ ID NO: 621, 421, 321, 721, 821, 921, 1021, 1121, or 1321), a FR-L2 comprising WYQQKPGQPPKLLIY (SEQ ID NO: 23, 623, 423, 323, 723, 823, 923, 1023, 1123, or 1323), a FR-L3 comprising GVPDRFSGSGSGTDFTLTISSLQAEDVAVYYC (SEQ ID NO: 25, 625, 425, 325, 725, 825, 925, 1025, 1125, or 1325), and a FR-L4 comprising FGGGTKVEIK (SEQ ID NO: 27, 627, 427, 327, 727, 827, 927, 1027, 1127, or 1327);
(II) (i) the FR-H1, FR-H2, FR-H3, FR-H4, FR-L1, FR-L2, FR-L3, and the FR-L4 contained in AMD-74967; or
(ii) a FR-H1 comprising QVQLVQSGAEVKKPGASVKVSCKASG (SEQ ID NO: 11 or 511), a FR-H2 comprising WVRQAPGQRLEWMG (SEQ ID NO: 13 or 513), a FR-H3 comprising RVTITRDTSASTAYMELSSLRSEDTAVYYC (SEQ ID NO: 515), a FR-H4 comprising WGQGTLVTVSS (SEQ ID NO: 17 or 517), a FR-L1 comprising GIVMTQSPDSLAVSLGERATINC (SEQ ID NO: 521), a FR-L2 comprising WYQQKPGQPPKLLIY (SEQ ID NO: 23 or 523), a FR-L3 comprising GVPDRFSGSGSGTDFTLTISSLQAEDVAVYYC (SEQ ID NO: 25 or 525), and a FR-LA comprising FGGGTKVEIK (SEQ ID NO: 27 or 527); or
(III) (i) the FR-H1, FR-H2, FR-H3, FR-H4, FR-L1, FR-L2, FR-L3, and the FR-L4 contained in ADI-74968, ADI-74966, ADI-74965, ADI-79842, ADI-79843, ADI-79848, ADI-79844, ADI-79845, or ADI-79847; or
(ii) a FR-H1 comprising QVQLVQSGAEVKKPGASVKVSCKASG (SEQ ID NO: 11 or 1211), a FR-H2 comprising WVRQAPGQRLEWMG (SEQ ID NO: 13 or 1213), a FR-H3 comprising RVTITRDTSASTAYMELSSLRSEDTAVYYC (SEQ ID NO: 1215), a FR-H4 comprising WGQGTLVTVSS (SEQ ID NO: 17 or 1217), a FR-L1 comprising DIVMTQSPDSLAVSLGERATINC (SEQ ID NO: 1221), a FR-L2 comprising WYQQKPGQPPKLLIY (SEQ ID NO: 1223), a FR-L3 comprising GVPDRFSGSGSGTDFTLTISSLQAEDVAVYYC (SEQ ID NO: 1225), and a FR-L4 comprising FGGGTKVEIK (SEQ ID NO: 1227).

10. The anti-CD3 antibody or antigen-binding fragment of any one of the foregoing claims, comprising:
(1) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74968; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 611, 612, 613, 614, 615, 616, 617, 621, 622, 623, 624, 625, 626, and 627, respectively;
(II) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74967; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 311, 512, 513, 514, 515, 516, 517, 521, 522, 523, 524, 525, 526, and 527, respectively:
(III) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74966; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 411, 412, 413, 414, 415, 416, 417, 421, 422, 423, 424, 425, 426, and 427, respectively;
(IV)(i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-74965; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 311, 312, 313, 314, 315, 316, 317, 321, 322, 323, 324, 325, 326, and 327, respectively;
(V) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79842; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS 711, 712, 713, 714, 715, 716, 717, 721, 722, 723, 724, 725, 726, and 727, respectively;
(VI) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79843; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 811, 812, 813, 814, 815, 816, 817, 821, 822, 823, 824, 825, 826, and 827, respectively;
(VII) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79848; or
(ii) a FR-H1, CDR-H1, FR-H2, CDR-H2, FR-H3, CDR-H3, FR-H4, FR-L1, CDR-1, FR-L2, CDR-L2, FR-L3, CDR-L3, and FR-L4 comprising SEQ ID NOS: 911, 912, 913, 914, 915, 916, 917, 921, 922, 923, 924, 925, 926, and 927, respectively;
(VIII) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79844; or
(ii) a FR-H1, CDR-H1, FR-H2, CDR-H2, FR-H3, CDR-H3, FR-L1, FR-L1, CDR-L1, FR-L2, CDR-L2, FR-L3, CDR-L3, and FR-L4 comprising SEQ ID NOS: 1011, 1012, 1013, 1014, 1015, 1016, 1017, 1021, 1022, 1023, 1024, 1025, 1026, and 1027, respectively;
(IX) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79845; or
(ii) a FR-H1, CDR-H1, FR-H2, CDR-H2, FR-H3, CDR-H3, FR-H1, FR-L1, CDR-L2, FR-L2, CDR-L2, FR-L3, CDR-L3, and FR-L4 comprising SEQ ID NOS: 1111, 1112, 1113, 1114, 1115, 1116, 1117, 1121, 1122, 1123, 1124, 1125, 1126, and 1127, respectively;
(X) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79846; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 1211, 1212, 1213, 1214, 1215, 1216, 1217, 1221, 1222, 1223, 1224, 1225, 1226, and 1227, respectively; or
(XI) (i) the FR-H1, CDR-H1, FR-H2, CDR-H2, FR-H3, CDR-H3, FR-H4, FR-L1, CDR-L1, FR-L2, CDR-L2, FR-L3, CDR-L3, and the FR-L4 contained in ADI-79847; or
(ii) a FR-H1, CDR-H1, FR-H2, CDR-H2, FR-H3, CDR-H3, FR-H4, FR-L1, CDR-L1, FR-L2, CDR-L2, FR-L3, CDR-L3, and FR-L4 comprising SEQ ID NOS: 1311, 1312, 1313, 1314, 1315, 1316, 1317, 1321, 1322, 1323, 1324, 1325, 1326, and 1327, respectively.

11. The anti-CD3 antibody or antigen-binding fragment of any one of the foregoing claims, comprising:
(A) a VH polypeptide comprising an amino acid sequence at least 80%, at least 85%, at least 90%, at least 92%, at least 94%, at least 95%, at least 96%, at least 97%, at least 98%, at least 99%, or 100% identical to SEQ ID NO 610, 510, 410, 310, 710, 810, 910, 1010, 1110, 1210, or 1310; and/or
(B) a VL polypeptide comprising the amino acid sequence at least 80%, at least 85%, at least 90%, at least 92%, at least 94%, at least 95%, at least 96%, at least 97%, at least 98%, at least 99%, or 100% identical to SEQ ID NO: 620, 520, 420, 320, 720, 820, 920, 1020, 1120, 1220, or 1320,
optionally wherein the anti-CD3 antibody or antigen-binding fragment comprises:
(I) (A) a VH polypeptide comprising (i) the (DR-H1, the CDR-H2, and the CFR-H3 sequences of SEQ ID NOS: 612, 614, and 616, respectively and (ii) an amino acid sequence at least 90% identical to SEQ ID NO: 610; and
(B) a VL polypeptide comprising (i) the CDR-L1, the CDR-L2, and the CFR-L3 sequences of SEQ ID NOS: 622, 624, and 626, respectively; and (ii) an amino acid sequence at least 90% identical to SEQ ID NO: 620; or
(II) (A) a VH polypeptide comprising (i) the CDR-H1, the CDR-H2, and the CDR-H3 sequences of SEQ ID NOS: 512, 514, and 516, respectively and (ii) an amino acid sequence at least 90% identical to SEQ ID NO: 510; and
(B) a VL polypeptide comprising (i) the CDR-L1, the CDR-L2, and the CFR-L3 sequences of SEQ ID NOS: 522, 524, and 526, respectively; and (ii) an amino acid sequence at least 90% identical to SEQ ID NO: 520.

12. The anti-CD3 antibody or antigen-binding fragment of any one of the foregoing claims, comprising VH and VL polypeptides comprising the amino acid sequences of:
(I) SEQ ID NOS: 610 and 620, respectively;
(II) SEQ ID NOS: 510 and 520, respectively;
(III) SEQ ID NOS: 410 and 420, respectively;
(IV) SEQ ID NOS: 310 and 320, respectively;
(V) SEQ ID NOS: 710 and 72′), respectively;
(VI) SEQ ID NOS: 810 and 820, respectively;
(VII) SEQ ID NOS: 910 and 920, respectively;
(VIII) SEQ ID NOS: 1010 and 1020, respectively;
(IX) SEQ ID NOS: 1110 and 1120, respectively;
(X) SEQ ID NOS: 1210 and 1220, respectively, or
(XI) SEQ ID NOS: 1310 and 1320, respectively.

13. The anti-CD3 antibody or antigen-binding fragment of any one of the foregoing claims, which comprises one or more of the following:
(i) an antibody constant region, a CH1 domain, a hinge, a CH2 domain, and/or a CH3 domain, optionally of or derived from an IgG or human IgG, further optionally of or derived from a human IgG1, IgG4, IgG2, or IgG3;
(ii) a fragment crystallizable (FR) region, optionally of:
(1) a human IgG1, further optionally comprising one or more of the following amino acid modifications: N297A, N297Q, D265A, 1234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, I332E, S298A, E333A, K334A, L234Y, L235Q, G236W, 5239M, H268D, D270E, K326D, A330M, K334E, G236A, K326W, S239D, E333S, S267E, H268F, S324T, E345R, E430G, S440Y M428L, N434S, L328F, M252Y, S254T, T256E, or any combination thereof, according to EU numbering;
(2) a human IgG4, further optionally comprising one or more of the following amino acid modifications: E233P, F234V, L235A, G237A, E318A, S228P, L236E, S241P, L248E, T394D, M252Y, S254T, T256E, N297A, N297Q, or arty combination thereof, according to E1 numbering;
(3) a human IgG2, further optionally comprising one or more of the following amino acid modifications: P238S, V234A, G237A, H268A, H268D, H268E, V309L, N297A, N297Q, A330S, P331S, C232S, C233S, M252Y, S254T, T256F, or any combination thereof, according to EU numbering; and/or
(4) a human IgG3, further optionally comprising E235Y, according to EU numbering;
(iii) an IgG, IgA, IgE, IgD, or IgM, optionally IgG1, IgG4, IgG2, or IgG3; and/or
(iv) an antibody fragment selected from the group consisting of: a fragment antigen-binding (Fab); an Fab₂; an Fab₃; an Fab′ fragment; an F(ab′)₂; a variable fragment (Fv); a single-chain Fv (scFv) fragment; a diabody; a triabody; a minibody; a scFv-Fc; a scFv2-Fc2; scFv-IgG; a monovalent IgG (or a half IgG); and/or a chimeric antigen receptor (CAR) comprising an antigen-binding region comprising said VH polypeptide and/or said VL polypeptide, a transmembrane domain, and at least one intracellular signaling domain (optionally derived from a T-cell receptor, further optionally CD3ξ).

14. The anti-CD3 antibody or antigen-binding fragment of any one of the foregoing claims, which comprises or is comprised in a multispecific antibody or antibody fragment having (a) a first antigen-binding region specific to CD3, comprising said VH polypeptide and/or said VL polypeptide, and (b) one or more other antigen-binding regions, optionally comprising one or more of the following features:
(i) the one or more other antigen-binding regions include an antigen-binding region that is specific to an oncology target, a target molecule expressed on cancer cells, an immune-oncology target, a target molecule expressed on immune cells, an autoimmune disorder target (optionally a self-reactive immune molecule or a target molecule expressed on an immune cell expressing a self-reactive immune molecule), an inflammatory disease target (optionally an inflammatory cytokine or chemokine or a receptor thereof), a neurodegenerative disease target, an infectious disease target (optionally a target molecule of a virus, bacterium, or a fungus), a target molecule expressed on infected cells (optionally infected with a virus, a bacterium, or fungus), a metabolic disease target, a cognitive disorder target, a blood-brain barrier target, or a blood disease target;
(ii) the one or more other antigen-binding regions include an antigen-binding region specific to a second antigen selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-4, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, RACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-JB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA 125, CAD-8, Calcitonin, cAMP, carcinoembryonic antigen (CEA), carcinoma-associated antigen (CAA), Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin 32/EphB4, EPO, ERCC, E-selectin, ET-1, factor IIa, Factor VII, Factor VIIIe, Factor IX, fibroblast activation protein (FAP), Fas, FcRI, FEN-1, Ferritin, FGF, FGF-19, FGF-2, FGF3, FGF-8, FGFR, FGFR-3, Fibrin, FL, FLIP, Fit-3, Fit-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HMI 24, HMFG PEM, HRG, Hrk, human cardiac myosin, human cytomegalovirus (HCMV), human growth hormone (HGH), HVEM, 1-309 antigen, TAP, ICAM, ICAM-1, ICAM-3, ICE, ICOS, IFNg, Ig, IgA receptor, IgE, IGP, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p05, PADPr, Parathyroid hormone, PARC, PARP, PBR, PBSF, PCAD, P-Cadherin, PCNA, PDGF, PDGF, PDK-1, PECAM, PEM, PF4, POE, PGF, PGI2, PGJ2, PIN, PLA2, placental alkaline phosphatase (PLAP), PIGF, PLP, PPI 4, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL RI Apo-2, DR4), TNFRSF10B (TRAIL R2 DR5, KILLER, TRICK-2A, TRICK-B), TNFRSF10C (TRAIL, R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF RI CD120a, p55-60), INFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1B13 CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL, Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSF1A (TNF-a Connectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD53), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VFM, Viral antigens, VLA, VLA-1, VLA-4, VNR, integrin, von Willebrand's factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), and hormone receptors and growth factors;
(iii) the one or more other antigen-binding regions include an antigen-binding region specific to an antigen selected from the group consisting of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcγRIIIa (CD16), FcγRIIa (CD32a), FcγRIIb (CD32b), FcγRI (CD64), Toll-like receptors (TLRs), TLR4, TLR9, cytokines, IL-2, IL-5, IL-43, IL-6, IL-47, IL-412, IL-23, TNFa, TGFb, cytokine receptors, IL-2R, chemokines, chemokine receptors, growth factors, VEGF, and HOF;
(iv) the multispecific antibody or antibody fragment is bispecific;
(v) the multispecific antibody or antibody fragment comprises at least three antigen-binding regions;
(vi) the multispecific antibody or antibody fragment is trispecific;
(vii) the multispecific antibody or antibody fragment comprises a multispecific format selected from the group consisting of: Fab-Fc-scFv, scFv2-Fc2, scFv-IgG, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab and/or
(viii) the multispecific antibody or antibody fragment comprises one or more of the following: at least one CLκ-preferring variant CH1 domain, optionally a CLκ-preferring variant CH1 domain described in WO2021067404; at least one CU-preferring variant CH1 domain, optionally a CLλ-preferring variant CH1 domain described in WO2021067404; at least one pair of a variant CH1 domain and a variant CL domain which preferentially pair with each other, optionally a pair described in WO2022150787; and/or at least one pair of a variant CH3 domain and another variant CH3 domain which preferentially pair with each other, optionally a pair described in WO2022150785.

15. The anti-CD3 antibody or antigen-binding fragment of any one of the foregoing claims, which comprises one or more of the following features:
(1) it binds to CD3 with a greater binding affinity or avidity at an acidic pH, optionally about pH 6.0, than at a physiological pH, optionally about pH 7.4, optionally wherein:
(i) the binding affinity or avidity is measured via:
(1) surface plasmon resonance (SPR), optionally using a BIACORE® system;
(2) bio-layer interferometry (BLI), optionally using an OCTET® system:
(3) enzyme-inked immunosorbent assay (ELISA); and/or
(4) radioimmunoassay (RIA);
(ii) the CD3 is:
(1) a human CD3, optionally CD3εδ; and/or
(2) a non-human primate CD3, optionally monkey CD3, further optionally cynomolgus CD3, optionally CD3εδ;
(iii) its binding to CD3 is at least ×1.2, at least ×1.5, at least ×2, at least ×5, at least ×10, at least ×20, at least ×50, at least ×100, at least ×10³, at least ×10⁴, at least ×10⁵ at least ×10⁶ at least ×10⁷, at least ×10⁸ or at least ×10⁹ greater at an acidic pH, optionally about pH 6.0, than at a physiological pH, optionally about pH 7.4, based on equilibrium dissociation constant (Kd) values; and/or
(iv) the anti-CD3 antibody or antigen-binding fragment thereof does not hind to CD3 at a physiological pH, optionally about pH 7.4;
(II) it binds to human CD3, optionally CD3εδ, at an acidic pH, optionally about pH 6.0:
(i) with a Kd value lower than 1.57×10⁸ (M), lower than 1.0×10⁸ (M), lower than 9.0×10⁹ (M), lower than 8.0×10⁹ (M), lower than 7.0×10⁹ (M), lower than 6.0×10⁹ (M), lower than 5.0×10⁹ (M), lower than 4.0×10⁹ (NM), lower than 3.0×10⁹ (M), lower than 2.0×10⁹ (M), lower than 1.0×10⁹ (M), lower than 9.0×10¹⁰ (M), or lower than 8.0×10¹⁰ (M), optionally measured via SPR, further optionally using a BIACORE® system;
(ii) with a Kd value between 1.56×10² (M) and 7.0×10¹⁰ (M), between 1.0×10⁸ (M) and 7.0×10¹⁰ (M), between 1.0×10⁸ (M) and 1.0×10⁹ (M), between 2.0×10⁸ (M) and 1.0×10⁹ (M), or between 5.0×10⁸ (MI) and 1.0×10⁹ (M), optionally measured via SPR, further optionally using a BIACORE® system;
(iii) with a Kd value lower than 9.33×10⁹ (M), lower than 9.0×10⁹ (M), lower than 8.0×10⁹ (M), lower than 7.0×10⁹ (M), lower than 6.0×10⁹ (M), lower than 5.0×10⁹ (M), lower than 4.0×10⁹ (M), lower than 3.0×10⁹ (M), or lower than 2.0×10⁹ (M), optionally measured via BLI, further optionally using an OCTET® system; and/or
(iv) with a Kd value between 9.32×10⁹ (M) and 1.0×10⁹ (M), between 9.0×10⁹ (M) and 1.0×10⁹ (M), between 8.0×10⁹ (M) and 3.0×10⁹ (M), between 7.0×10⁹ (M) and 4.0×10⁹, or between 6.0×10⁹ (M) and 5.0×10⁹ (M), optionally measured via BLI, further optionally using an OCTET® system;
(III) it binds to cynomolgus CD3, optionally CD3εδ, at an acidic pH, optionally about pH 6.0:
(i) optionally with a Kd value lower than 2.0×10⁸ (M), lower than 1.0×10⁸ (M), lower than 9.0×10⁹ (M), lower than 8.0×10⁹ (M), lower than 7.0×10⁹ (M), low er than 6.0×10⁹ (M), lower than 5.0×10⁹ (M), lower than 4.0×10⁹ (M), lower than 3.0×10⁹ (M), or lower than 2.0×10⁹ (M), optionally measured via BLI, further optionally using an OCTET® system; and/or
(ii) optionally with a Kd value between 2.0×10⁹ (M) and 1.0×10⁹ (M), between 1.0×10⁸ (M) and 1.0×10⁹ (M), between 9.0×10⁹ (M) and 2.0×10⁹ (M), or between 8.0×10⁹ (M) and 5.0×10⁹, optionally measured via BLI, further optionally using an OCTET® system;
(IV) it binds to CD3-expressing cells with greater binding at an acidic pH, optionally about pH 6.0, than at a physiological pH, optionally about pH 7.4, optionally wherein:
(i) the binding is measured via flow cytometry, further optionally based on a normal cell binding (NCB) value calculated using median fluorescence intensity (MFI) values,
(ii) the cells express:
(1) a human CD3, optionally CD3εδ; and/or
(2) a non-human primate CD3, optionally monkey CD3, further optionally cynomolgus CD3, optionally CD3εδ;
(iii) the cells are:
(1) primary cells;
(2) cells of a cell line;
(3) human cells, optionally human T cells, further optionally Jurkat cells;
(4) non-human primate cells, optionally monkey cells, further optionally cynomolgus cells, yet further optionally HSC-F cells,
(iv) binding to CD3-expressing cells is at least ×1.2, at least ×1.5, at least ×2, at least ×3, at least ×4, at least ×5, at least ×10, at least ×20, at least ×50, at least ×100, at least ×10³, at least ×10⁴, at least ×10⁵, at least ×10⁶, at least ×10⁷, at least ×10⁸, or at least ×10⁹ greater at an acidic pH, optionally about pH 6.0, than at a physiological pH, optionally about pH 7.4, based on NCB values when measured via flow cytometry; and/or
(v) the anti-CD3 antibody or antigen-binding fragment thereof does not bind to CD3-expressing cells at a physiological p1H, optionally about pH 7.4
(V) upon binding to CD3 on cells, optionally T cells, it elicits activation and/or enhances cell cytotoxic function(s) of the cells;
(VI) upon binding to CD3 on cells, optionally T cells, it does not elicit cytokine production upon binding to CD3 to levels capable of inducing cytokine release syndrome (CRS); and/or
(VII) it comprises or is comprised in a multispecific antibody or antibody fragment having at least (a) a first antigen-binding region specific to CD3, comprising said VH polypeptide and/or said VL polypeptide, and (b) one or more other antigen-binding regions which include at least one antigen-binding region which binds to a different antigen than the first antigen-binding region; and upon binding to (i) CD3 on a first cell, optionally T cells, and (ii) the second antigen expressed on a second cell, the first cell exhibits cytotoxicity to the second cell.

16. A nucleic acid encoding the anti-CD3 antibody or antigen-binding fragment of any one of the foregoing claims, optionally comprising:
(A) a VEH polypeptide-encoding nucleic acid sequence at least 80%, at least 85%, at least 90%, at least 92%, at least 94%, at least 95%, at least 96%, at least 97%, at least 98%, at least 99%, or 100% identical to SEQ ID NO: 650, 550, 450, 350, 750, 850, 950, 1050, 1150, 1250, or 1350; and/or
(B) a VL polypeptide-encoding nucleic ai id sequence at least 80%, at least 85%, at least 90%, at least 92%, at least 94%, at least 95%, at least 96%, at least 97%, at least 98%, at least 99%, or 100% identical to SEQ ID NO: 660, 560, 460, 360, 760, 860, 960, 1060, 1160, 1260, or 1360,
further optionally comprising VH polypeptide-encoding and VL polypeptide-encoding nucleic acid sequences of:
(I) SEQ ID NOS: 650 and 660, respectively;
(II) SEQ ID NOS: 550 and 560, respectively;
(III) SEQ ID NOS: 450 and 460, respectively;
(IV) SEQ ID NOS: 350 and 360, respectively;
(V) SEQ ID NOS: 750 and 760, respectively;
(VI) SEQ ID NOS: 850 and 860, respectively;
(VII) SEQ ID NOS: 950 and 960, respectively;
(VIII) SEQ ID NOS: 1050 and 1060, respectively;
(IX) SEQ ID NOS: 1150 and 1160, respectively;
(X) SEQ ID NOS: 1250 and 1260, respectively; or
(XI) SEQ ID NOS: 1350 and 1360, respectively.

17. A vector comprising the nucleic acid of claim 16, optionally wherein:
(i) the vector is an expression vector, and/or
(ii) the vector comprises a plasm id, a viral vector (optionally adenoviral, lentiviral, or retroviral), a lipid-based vector, a self-replicating RNA vector, a virus-like particle, a polymer-based vector, and/or a nanoparticle, optionally a lipid-based nanoparticle.

18. An isolated or recombinant cell comprising, transfected with, transformed with, or transduced with the nucleic acid of claim 16 or the vector of claim 17, optionally wherein the isolated or recombinant cell is:
(i) mammalian, optionally human, non-human primate, monkey, rabbit, rodent, hamster, rat, or mouse; or
(ii) non-mammalian, optionally plant, bacterial, fungal, yeast, protozoa, or insect, and optionally wherein the isolated or recombinant cell is an immune cell or a hybridoma.

19. A pharmaceutical composition comprising:
(A) the anti-CD3 antibody or antigen-binding fragment of any one of claims 1-15, the nucleic acid of claim 16, the vector of claim 17, and/or the isolated or recombinant cell claim 18; and
(B) a pharmaceutically acceptable carrier and/or excipient.

20. A method of treating a subject in need of such treatment, comprising administering to the subject an effective amount of:
(i) the anti-CD3 antibody or antigen-binding fragment of any one of claims 1-15;
(iii) the nucleic acid of claim 16;
(iii) the vector of claim 17;
(iv) the isolated or recombinant cell of claim 18, and/or
(v) the pharmaceutical composition of claim 19,
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent.

21. A method of treating or preventing a disease, disorder, or a condition in a subject, comprising administering to the subject an effective amount of:
(i) the anti-CD3 antibody or antigen-binding fragment of any one of claims 1-15;
(ii) the nucleic acid of claim 16;
(iii) the vector of claim 17;
(iv) the isolated or recombinant cell of claim 18, and/or
(v) the pharmaceutical composition of claim 19,
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile; and/or
(b) the method further comprises administering to the subject an additional agent, optionally an adjuvant or a therapeutic agent.

22. A method of eliciting cytotoxicity to a cell expressing a target molecule of interest, comprising administering to the subject art effective amount of:
(i) the anti-CD3 antibody or antigen-binding fragment of claim 14;
(ii) a nucleic acid encoding the anti-CD3 antibody or antigen-binding fragment of claim 14;
(iii) a vector comprising the nucleic acid of (ii);
(iv) isolated or recombinant cell comprising, transfected with, transformed with, or transduced with the nucleic acid or the vector; and/or
(v) a pharmaceutical composition comprising (A) the anti-CD3 antibody or antigen-binding fragment of claim 14, a nucleic acid encoding the anti-CD3 antibody or antigen-binding fragment of claim 14, a vector comprising the nucleic acid, and/or a cell comprising, transfected with, transformed with, or transduced with the nucleic acid or the vector; and/or (B) a pharmaceutically acceptable carrier and/or excipient,
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent.

23. The method of any one of claims 20-22, the disease, disorder, or a condition comprises cancer or a neoplastic condition, an autoimmune disease, a neurodegenerative disease, art infectious disease, an inflammatory disease, or another disease, optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof, and/or
(i-2) a liquid cancer, optionally chosen from: chronic lymphocytic leukemia (CLL), mantle cell lymphoma (MC L), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndromes non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmacytoma of bone, extraosseous plasmacytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type 1 diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjorgen's syndrome, Addison's disease, Behcet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus (MRSA), Clostridium difficile, Streptococcus pneumoniae, Klebsiella pneumoniae, Pseudomonas aeruginosa, Helicobacter pylori, Neisseria gonorrhoeae, Vibrio, vulnificus, and/or (3) the fungal disease is Aspergillosis, Candida, Candida auris, Cryptococcus neoformans, Pneumocystis jirovecii, Mucoromycetes, Talaromyces, ringworm, Blastomyces, Coccidioides, Cryptococcus gattii, Histoplasma, Paracoccidioides, or Sporothrix infection.

24. A method of manufacturing the anti-CD3 antibody or antigen-binding fragment of any one of claims 1-15, comprising:
(a) culturing cells comprising the nucleic acid of claim 16 in a condition that allows for expression of said antibody or antigen-binding fragment, and
(b) harvesting and purifying the antibody or antigen-binding fragment from the cell culture from (a).

25. A method of manufacturing the isolated or recombinant cell of claim 18 or the population of such cells, comprising introducing the nucleic acid of claim 16 and/or the vector of claim 17 into one or more cells, optionally wherein the introducing occurs in vitro, ex vivo, or in vivo.

26. The anti-CD3 antibody or antigen-binding fragment of any one of claims 1-15, the nucleic acid of claim 16, the vector of claim 17, the cell of claim 18 or a population of such cells, and/or the pharmaceutical composition of claim 19, for use in medicine.

27. The anti-CD3 antibody or antigen-binding fragment of any one of claims 1-15, the nucleic acid of claim 16, the vector of claim 17, the cell of claim 18 or a population of such cells, and/or the pharmaceutical composition of claim 19, for use in treating a disease, disorder, or condition, optionally wherein the disease, disorder, or condition comprises cancer or a neoplastic condition, an autoimmune disease, a neurodegenerative disease, an infectious disease, an inflammatory disease, or another disease, further optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof; and/or
(i-2) a liquid cancer, optionally chosen from: chronic lymphocytic leukemia (CLL), mantle cell lymphoma (MCL), multiple myeloma, acute lymphoid leukemia (ALL), Hodgkin lymphoma, B-cell acute Lymphoid leukemia (BALL), i-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmacytoma of bone, extraosseous plasmacytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type I diabetes, systemic lupus erythematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren's syndrome, Addison's disease, Behcet's disease, Schmidt syndrome, celiac disease, dermatomyositis, autoimmune vitiligo, Graves' disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, Influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, meticillin-resistant Staphylococcus aureus (MRSA), Clostridium dificile, Streptococcus pneumoniae, Klebsiella pneumoniae, Pseudomonas aeruginosa, Helicobacter pylori, Neisseria gonorrhoeae, Vibrio vulnificus, and/or (3) the fungal disease is Aspergillosis, Candida, Candida auris, Cryptococcus neoformans, Pneumocystis jirovecii, Mucormycoses, Talaromyces, ringworm, Blastomyces, Coccidioides, Cryptococcus gattii, Histoplasma, Paracoccidioides, or Sporothrix infection.

28. Use of the anti-CD3 antibody or antigen-binding fragment of any one of claims 1-15, the nucleic acid of claim 16, the vector of claim 17, the isolated or recombinant cell of claim 18 or a population of such cells, and/or the pharmaceutical composition of claim 19 for the manufacture of a medicament for treatment of a disease, disorder, or condition, optionally wherein the disease, disorder, or condition comprises cancer or a neoplastic condition, an autoimmune disease, a neurodegenerative disease, an infectious disease, sin inflammatory disease, or another disease, further optionally wherein:
(i) the cancer is:
(i-1) a solid cancer, optionally chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof; and/or
(i-2) a liquid cancer, optionally chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmacytoma of bone, extraosseous plasmacytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma;
(ii) the autoimmune or inflammatory disease is psoriasis, rheumatoid arthritis, autoimmune arthritis, type I diabetes, sarcoidosis, systemic lupus erythrematosus, myasthenia gravis, multiple sclerosis, scleroderma, inflammatory bowel disease, Crohn's disease, ulcerative colitis, Guillain-Barre syndrome, chronic inflammatory demyelinating polyneuropathy, pemphigus vulgaris, Sjogren's syndrome, Addison's disease, Behcet's disease, Schmidt syndrome, celiac disease, dermatomyositis, autoimmune vitiligo, Graves disease, Hashimoto thyroiditis, Kawasaki disease, pernicious anemia, autoimmune vasculitis, or fibrosis;
(iii) the neurodegenerative disease is Alzheimer's disease, Huntington's disease, Parkinson's disease, amyotrophic lateral sclerosis, Friedreich ataxia, Lewy body disease, spinal muscular atrophy, motor neuron disease, multiple sclerosis, Batten disease, Creutzfeldt-Jakob disease;
(iv) the infectious disease is a viral, bacterial, fungal, yeast, protozoan, prion or parasitic disease, optionally wherein (1) the viral disease is human immunodeficiency virus (HIV), hepatitis virus (optionally hepatitis A, B, or C virus), human papillomavirus (HPV), herpes simplex virus (HSV) (optionally HSV-1 or HSV-2), enterovirus, human cytomegalovirus, adenovirus, rhinovirus, Pox virus, influenza virus, coronavirus (optionally MERS-CoV, SARS-CoV, or SARS-CoV-2, or common human coronavirus), norovirus, West Nile Virus, Zika virus, poliovirus, Ebola virus, or dengue virus (DENV) infection, (2) the bacterial disease is Salmonella, Escherichia coli, Mycobacterium tuberculosis, methicillin-resistant Staphylococcus aureus MRSA), Clostridium difficile, Streptococcus pneumoniae, Klebsiella pneumoniae, Pseudomonas aeruginosa, Helicobacter pylori, Neisseria gonorrhoeae, Vibrio vulnificus, and/or (3) the fungal disease is Aspergillosis, Candida, Candida auris, Cryptococcus neoformans, Pneumocystis jirovecii, Mucoromycetes, Talaromyces, ringworm, Blastomyces, Coccidioides, Cryptococcus gattii, Histoplasma, Paracoccidioides, or Sporothrix infection.
